SEQUENCE LISTING (39 sequences)

SEQ ID NO: 1 - RNA, 106 nt, Dengue virus type 2
cagcauauugacgcugggaaagaccagagauccugcugucuccucagcaucauuccaggcacagaacgccagaaaauggaauggugcuguugaaucaacagguucu

SEQ ID NO: 2 - RNA, 70 nt, Dengue virus type 2
uaaaagucaggucggaucaagccauaguacggaaaaaacuaugcuaccugugagccccguccaaggacgu

SEQ ID NO: 3 - RNA, 67 nt, Dengue virus type 2
gucaggccaucacaaaugccacagcuugaguaaacugugcagccuguagcuccaccugagaaggugu

SEQ ID NO: 4 - RNA, 67 nt, Dengue virus type 2
ggaggccacaaaccauggaagcuguacgcauggcguaguggacuagcgguuagaggagaccccuccc

SEQ ID NO: 5 - RNA, 66 nt, Dengue virus type 2
gggggcccaaggugagaugaagcuguagucucacuggaaggacuagagguuagaggagaccccccc

SEQ ID NO: 6 - RNA, 67 nt, Dengue virus type 2
ggaggccacaaaccauggaagcuguacgcauggcguagugaacuagcaguuagaggagaccccuccc

SEQ ID NO: 7 - RNA, 67 nt, Dengue virus type 2
ggaggccacaaaccauggaagcuguacgcauggcguaguggacuagcgguuagacgacaccccuccc

SEQ ID NO: 8 - DNA, 462 bp, Dengue virus type 1
gccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 9 - DNA, 451 bp, Dengue virus type 2
aaagcaaaactaacatgaaacaaggctagaagtcaggtcggattaagccatagtacggaaaaaactatgctacctgtgagccccgtccaaggacgttaaaagaagtcaggccatcataaatgccatagctggagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 10 - DNA, 440 bp, Dengue virus type 3
acgtaggaagtgaaaaagaggcaaactgtcaggccaccttaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaacaaaaacagcatattgacgctgggagagaccagagatcctgctgtctcctcagcatcattccaggcacagaacgccagaaaatggaatggtgctgttgaatcaacaggttct

SEQ ID NO: 11 - DNA, 385 bp, Dengue virus type 4
ttaccaacaacaaacaccaaaggctattgaagtcaggccacttgtgccacggtttgagcaaaccgtgctgcctgtagctccgccaacaatgggaggcgtaataatccccagggaggccatgcgccacggaagctgtacgcgtggcatattggactagcggttagaggagacccctcccatcactgacaaaacgcagcaaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 12 - DNA, 575 bp, Japanese encephalitis virus
tgtgatttaaggtagaaaagtagactatgtaaataatgtaaatgagaaaatgcatgcatatggagtcaggccagcaaaagctgccaccggatactgggtagacggtgctgcctgcgtctcagtcccaggaggactgggttaacaaatctgacaacagaaagtgagaaagccctcagaaccgtctcggaagtaggtccctgctcactggaagttgaaagaccaacgtcaggccacaaatttgtgccactccgctagggagtgcggcctgcgcagccccaggaggactgggttaccaaagccgttgagcccccacggcccaagcctcgtctaggatgcaatagacgaggtgtaaggactagaggttagaggagaccccgtggaaacaacaacatgcggcccaagccccctcgaagctgtagaggaggtggaaggactagaggttagaggagaccccgcatttgcatcaaacagcatattgacacctgggaatagactgggagatcttctgctctatctcaacatcagctactaggcacagagcgccgaagtatgtagctggtggtgaggaagaacac

SEQ ID NO: 13 - DNA, 614 bp, Murray Valley encephalitis virus
ataacattgatagaaaattttgtaaatatttaatgtaatatagtataggtaaaattttttgaaattaagtaaaattaagtagcaagacttgatagtcaggccagccggttaggctgccaccgaaggttggtagacggtgctgcctgcgaccaaccccaggaggactgggttaccaaagctgattctccacggttggaaagcctcccagaaccgtctcggaagaggagtccctgccaacaatggagatgaagcccgtgtcagatcgcgaaagcgccacttcgccgaggagtgcaatctgtgaggccccaggaggactgggtaaacaaagccgtaaggcccccgcagcccgggccgggaggaggtgatgcaaaccccggcgaaggactagaggttagaggagaccctgcggaagaaatgagtggcccaagctcgccgaagctgtaaggcgggtggacggactagaggttagaggagaccccactctcaaaagcatcaaacaacagcatattgacacctgggaaaagactaggagatcttctgctctattccaacatcagtcacaaggcaccgagcgccgaacactgtgactgatgggggagaagaccacaggatct

SEQ ID NO: 14 - DNA, 292 bp, Powassan virus
actagcacaaccaaataatcagaagaaacccactacagggggtgatgtggcagcgcaccacgacatcgtgacgggaagaggtcgtccccgacgcatcatctctctagggcattttcgtgagaccctcatggctgctaaaggggcatcagccgtgtagtaagaaggccccgacctactgcagcagcacacacagtgacgggaagttggtcgctcccgacgcagttaggtcagagaaaattttgtgagaccaaaaggcctcctggaaggctcaccaggggttaggccattctag

SEQ ID NO: 15 - DNA, 549 bp, St. Louis encephalitis virus
attgaaatgtaaatagtgtaaataaataaaaacaggtaagtcaggccaatcagttttgccaccggatgtcaggtaaacggtgctgtctgtaacctggccccaggcgactgggttatcaaagccaacccggctgggtgcaaagcccctcattccgactcgggagggtccctggcacgtaggctggagaggacgcacaagtcagaccagaaatgccacctgaaagcatgctaaaggtgctgtctgtacatgccccaggaggactgggttaacaaagcttaacagccccagcggcccaaaccatggagtgcgtgaccatggcgtaaggactagaggttagaggagaccccgctgtaacttggcaaggcccaaacccgctcaaagctgtagagacgggggaaggactagaggttagaggagaccccttgccgttaacgcaaacaacagcatattgacacctggaaagacaggagatcccctgctttttcaacaccagccacaaggcacagagcgccgtaaagtgtggctggtggtgaaaaaatcacaggatct

SEQ ID NO: 16 - DNA, 764 bp, Tick-borne encephalitis virus
acccagactgtgacagagcaaaacccggaaggctcgtaaaagattgtccggaaccaaaagaaaagcaagcaactcacagagatagagctcggactggagagctctttaaacaaaaaaaaaaaaaaaaaaaaaaaaaaaaaaaaaaaaaaaaaaaaaaaaagccagaattgagctgaacctggagagctcattaaatacagtccagacgaaacaaaacatgacaaagcaaagaggctgagctaaaagttcccactacgggactgcttcatagcggtttgtggggggaggctaggaggcgaagccacagatcatggaatgatgcggcagcgcgcgagagcgacggggaagtggtcgcacccgacgcaccatccatgaagcaatacttcgtgagaccccccctgaccagcaaagggggcagaccggtcaggggtgaggaatgcccccagagtgcattacggcagcacgccagtgagagtggcgacgggaaaatggtcgatcccgacgtagggcactctgaaaaattttgtgagaccccctgcatcatgataaggccgaacatggtgcatgaaaggggaggcccccggaagcacgcttccgggaggagggaagagagaaattggcagctctcttcaggatttttcctcctcctatacaaaattccccctcggtagagggggggcggttcttgttctccctgagccaccatcacccagacacaggtagtctgacaaggaggtgatgtgtgactcggaaaaacacccgct

SEQ ID NO: 17 - DNA, 573 bp, West Nile virus
aagatagtattatagttagtttagtgtaaataggatttattgagaatggaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacatcgccccacgcggccataaccctggctatggtgttaaccagggagaagggactagaggttagaggagaccccgcgtaaaaaagtgcacggcccaacttggctgaagctgtaagccaagggaaggactagaggttagaggagaccccgtgccaaaaacaccaaaagaaacagcatattgacacctgggatagactaggggatcttctgctctgcacaaccagccacacggcacagtgcgccgacataggtggctggtggtgctagaacacaggatct

SEQ ID NO: 18 - DNA, 428 bp, Flavivirus FSME
gcaccaatcttagtgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 19 - DNA, 508 bp, Yellow fever virus
aacaccatctaacaggaataaccgggatacaaaccacgggtggagaaccggactccccacaacctgaaaccgggatataaaccacggctggagaaccggactccgcacttaaaatgaaacagaaaccgggataaaaactacggatggagaaccggactccacacattgagacagaagaagttgtcagcccagaaccccacacgagttttgccactgctaagctgtgaggcagtgcaggctgggacagccgacctccaggttgcgaaaaacctggtttctgggacctcccaccccagagtaaaaagaacggagcctccgctaccaccctcccacgtggtggtagaaagacggggtctagaggttagaggagaccctccagggaacaaatagtgggaccatattgacgccagggaaagaccggagtggttctctgcttttcctccagaggtctgtgagcacagtttgctcaagaataagcagacctttggatgacaaacacaaaaccact

SEQ ID NO: 20 - DNA, 10735 bp, Dengue virus type 1
agttgttagtctacgtggaccgacaagaacagtttcgaatcggaagcttgcttaacgtagttctaacagttttttattagagagcagatctctgatgaacaaccaacggaaaaagacgggtcgaccgtctttcaatatgctgaaacgcgcgagaaaccgcgtgtcaactgtttcacagttggcgaagagattctcaaaaggattgctttcaggccaaggacccatgaaattggtgatggcttttatagcattcctaagatttctagccatacctccaacagcaggaattttggctagatggggctcattcaagaagaatggagcgatcaaagtgttacggggtttcaagaaagaaatctcaaacatgttgaacataatgaacaggaggaaaagatctgtgaccatgctcctcatgctgctgcccacagccctggcgttccatctgaccacccgagggggagagccgcacatgatagttagcaagcaggaaagaggaaaatcacttttgtttaagacctctgcaggtgtcaacatgtgcacccttattgcaatggatttgggagagttatgtgaggacacaatgacctacaaatgcccccggatcactgagacggaaccagatgacgttgactgttggtgcaatgccacggagacatgggtgacctatggaacatgttctcaaactggtgaacaccgacgagacaaacgttccgtcgcactggcaccacacgtagggcttggtctagaaacaagaaccgaaacgtggatgtcctctgaaggcgcttggaaacaaatacaaaaagtggagacctgggctctgagacacccaggattcacggtgatagccctttttctagcacatgccataggaacatccatcacccagaaagggatcatttttattttgctgatgctggtaactccatccatggccatgcggtgcgtgggaataggcaacagagacttcgtggaaggactgtcaggagctacgtgggtggatgtggtactggagcatggaagttgcgtcactaccatggcaaaagacaaaccaacactggacattgaactcttgaagacggaggtcacaaaccctgccgtcctgcgcaaactgtgcattgaagctaaaatatcaaacaccaccaccgattcgagatgtccaacacaaggagaagccacgctggtggaagaacaggacacgaactttgtgtgtcgacgaacgttcgtggacagaggctggggcaatggttgtgggctattcggaaaaggtagcttaataacgtgtgctaagtttaagtgtgtgacaaaactggaaggaaagatagtccaatatgaaaacttaaaatattcagtgatagtcaccgtacacactggagaccagcaccaagttggaaatgagaccacagaacatggaacaactgcaaccataacacctcaagctcccacgtcggaaatacagctgacagactacggagctctaacattggattgttcacctagaacagggctagactttaatgagatggtgttgttgacaatggaaaaaaaatcatggctcgtccacaaacaatggtttctagacttaccactgccttggacctcgggggcttcaacatcccaagagacttggaatagacaagacttgctggtcacatttaagacagctcatgcaaaaaagcaggaagtagtcgtactaggatcacaagaaggagcaatgcacactgcgttgactggagcgacagaaatccaaacgtctggaacgacaacaatttttgcaggacacctgaaatgcagactaaaaatggataaactgactttaaaagggatgtcatatgtaatgtgcacagggtcattcaagttagagaaggaagtggctgagacccagcatggaactgttctagtgcaggttaaatacgaaggaacagatgcaccatgcaagatccccttctcgtcccaagatgagaagggagtaacccagaatgggagattgataacagccaaccccatagtcactgacaaagaaaaaccagtcaacattgaagcggagccaccttttggtgagagctacattgtggtaggagcaggtgaaaaagctttgaaactaagctggttcaagaagggaagcagtatagggaaaatgtttgaagcaactgcccgtggagcacgaaggatggccatcctgggagacactgcatgggacttcggttctataggaggggtgttcacgtctgtgggaaaactgatacaccagatttttgggactgcgtatggagttttgttcagcggtgtttcttggaccatgaagataggaatagggattctgctgacatggctaggattaaactcaaggagcacgtccctttcaatgacgtgtatcgcagttggcatggtcacgctgtacctaggagtcatggttcaggcggactcgggatgtgtaatcaactggaaaggcagagaactcaaatgtggaagcggcatttttgtcaccaatgaagtccacacctggacagagcaatataaattccaggccgactcccctaagagactatcagcggccattgggaaggcatgggaggagggtgtgtgtggaattcgatcagccactcgtctcgagaacatcatgtggaagcaaatatcaaatgaattaaaccacatcttacttgaaaatgacatgaaatttacagtggtcgtaggagacgttagtggaatcttggcccaaggaaagaaaatgattaggccacaacccatggaacacaaatactcgtggaaaagctggggaaaagccaaaatcataggagcagatgtacagaataccaccttcatcatcgacggcccaaacaccccagaatgccctgataaccaaagagcatggaacatttgggaagttgaagactatggatttggaattttcacgacaaacatatggttgaaattgcgtgactcctacactcaagtgtgtgaccaccggctaatgtcagctgccatcaaggatagcaaagcagtccatgctgacatggggtactggatagaaagtgaaaagaacgagacttggaagttggcaagagcctccttcatagaagttaagacatgcatctggccaaaatcccacactctatggagcaatggagtcctggaaagtgagatgataatcccaaagatatatggaggaccaatatctcagcacaactacagaccaggatatttcacacaaacagcagggccgtggcacttgggcaagttagaactagattttgatttatgtgaaggtaccactgttgttgtggatgaacattgtggaaatcgaggaccatctcttagaaccacaacagtcacaggaaagacaatccatgaatggtgctgtagatcttgcacgttaccccccctacgtttcaaaggagaagacgggtgctggtacggcatggaaatcagaccagtcaaggagaaggaagagaacctagttaagtcaatggtctctgcagggtcaggagaagtggacagtttttcactaggactgctatgcatatcaataatgatcgaagaggtaatgagatccagatggagcagaaaaatgctgatgactggaacattggctgtgttcctccttctcacaatgggacaattgacatggaatgatctgatcaggctatgtatcatggttggagccaacgcttcagacaagatggggatgggaacaacgtacctagctttgatggccactttcagaatgagaccaatgttcgcagtcgggctactgtttcgcagattaacatctagagaagttcttcttcttacagttggattgagtctggtggcatctgtagaactaccaaattccttagaggagctaggggatggacttgcaatgggcatcatgatgttgaaattactgactgattttcagtcacatcagctatgggctaccttgctgtctttaacatttgtcaaaacaactttttcattgcactatgcatggaagacaatggctatgatactgtcaattgtatctctcttccctttatgcctgtccacgacttctcaaaaaacaacatggcttccggtgttgctgggatctcttggatgcaaaccactaaccatgtttcttataacagaaaacaaaatctggggaaggaaaagctggcctctcaatgaaggaattatggctgttggaatagttagcattcttctaagttcacttctcaagaatgatgtgccactagctggcccactaatagctggaggcatgctaatagcatgttatgtcatatctggaagctcggccgatttatcactggagaaagcggctgaggtctcctgggaagaagaagcagaacactctggtgcctcacacaacatactagtggaggtccaagatgatggaaccatgaagataaaggatgaagagagagatgacacactcaccattctcctcaaagcaactctgctagcaatctcaggggtatacccaatgtcaataccggcgaccctctttgtgtggtatttttggcagaaaaagaaacagagatcaggagtgctatgggacacacccagccctccagaagtggaaagagcagtccttgatgatggcatttatagaattctccaaagaggattgttgggcaggtctcaagtaggagtaggagtttttcaagaaggcgtgttccacacaatgtggcacgtcaccaggggagctgtcctcatgtaccaagggaagagactggaaccaagttgggccagtgtcaaaaaagacttgatctcatatggaggaggttggaggtttcaaggatcctggaacgcgggagaagaagtgcaggtgattgctgttgaaccggggaagaaccccaaaaatgtacagacagcgccgggtaccttcaagacccctgaaggcgaagttggagccatagctctagactttaaacccggcacatctggatctcctatcgtgaacagagagggaaaaatagtaggtctttatggaaatggagtggtgacaacaagtggtacctacgtcagcgccatagctcaagctaaagcatcacaagaagggcctctaccagagattgaggacgaggtgtttaggaaaagaaacttaacaataatggacctacatccaggatcggggaaaacaagaagatatcttccagccatagtccgtgaggccataagaaggaacgtgcgcacgctagtcttagctcccacaagagttgtcgcttctgaaatggcagaggcgctcaagggaatgccaataaggtatcagacaacagcagtgaagagtgaacacacaggaaaagagatagttgaccttatgtgtcacgccactttcactatgcgtctcctgtctcctgtgagagttcccaattataatatgattatcatggatgaagcacattttaccgatccagccagcatagcagccagagggtatatctcaacccgagtgggtatgggtgaagcagctgcgattttcatgacagccactccccccggatcggtggaggcctttccacagagcaatgcagttatccaagatgaggaaagagacattcctgaaagatcatggaactcaggctatgactggatcactgatttcccaggtaaaacagtctggtttgttccaagcatcaaatcaggaaatgacattgccaactgtttaagaaagaatgggaaacgggtggtccaattgagcagaaaaacttttgacactgagtaccagaaaacaaaaaataacgactgggactatgttgtcacaacagacatatccgaaatgggagcaaacttccgagccgacagggtaatagacccgaggcggtgcctgaaaccggtaatactaaaagatggcccagagcgtgtcattctagccggaccgatgccagtgactgtggctagcgccgcccagaggagaggaagaattggaaggaaccaaaataaggaaggcgatcagtatatttacatgggacagcctctaaacaatgatgaggaccacgcccattggacagaagcaaaaatgctccttgacaacataaacacaccagaagggattatcccagccctctttgagccggagagagaaaagagtgcagcaatagacggggaatacagactacggggtgaagcgaggaaaacgttcgtggagctcatgagaagaggagatctacctgtctggctatcctacaaagttgcctcagaaggcttccagtactccgacagaaggtggtgctttgatggggaaaggaacaaccaggtgttggaggagaacatggacgtggagatctggacaaaagaaggagaaagaaagaaactacgaccccgctggctggatgccagaacatactctgacccactggctctgcgcgaattcaaagagttcgcagcaggaagaagaagcgtctcaggtgacctaatattagaaatagggaaacttccacaacatttaacgcaaagggcccagaacgccttggacaatctggttatgttgcacaactctgaacaaggaggaaaagcctatagacacgccatggaagaactaccagacaccatagaaacgttaatgctcctagctttgatagctgtgctgactggtggagtgacgttgttcttcctatcaggaaggggtctaggaaaaacatccattggcctactctgcgtgattgcctcaagcgcactgctatggatggccagtgtggaaccccattggatagcggcctctatcatactggagttctttctgatggtgttgcttattccagagccggacagacagcgcactccacaagacaaccagctagcatacgtggtgataggtctgttattcatgatattgacagcggcagccaatgagatgggattactggaaaccacaaagaaggacctggggattggtcatgcagctgctgaaaaccaccatcatgctgcaatgctggacgtagacctacatccagcttcagcctggactctctatgcagtggccacaacaattatcactcccatgatgagacacacaattgaaaacacaacggcaaatatttccctgacagctattgcaaaccaggcagctatattgatgggacttgacaagggatggccaatatcaaagatggacataggagttccacttctcgccttggggtgctattctcaggtgaacccgctgacgctgacagcggcggtatttatgctagtggctcattatgccataattggacccggactgcaagcaaaagctactagagaagctcaaaaaaggacagcagccggaataatgaaaaacccaactgtcgacgggatcgttgcaatagatttggaccctgtggtttacgatgcaaaatttgaaaaacagctaggccaaataatgttgttgatactttgcacatcacagatcctcctgatgcggaccacatgggccttgtgtgaatccatcacactagccactggacctctgactacgctttgggagggatctccaggaaaattctggaacaccacgatagcggtgtccatggcaaacatttttaggggaagttatctagcaggagcaggtctggccttttcattaatgaaatctctaggaggaggtaggagaggcacgggagcccaaggggaaacactgggagaaaaatggaaaagacagctaaaccaattgagcaagtcagaattcaacacttacaaaaggagtgggattatagaggtggatagatctgaagccaaagaggggttaaaaagaggagaaccgactaaacacgcagtgtcgagaggaacggccaaactgaggtggtttgtggagaggaaccttgtgaaaccagaagggaaagtcatagacctcggttgtggaagaggtggctggtcatattattgcgctgggctgaagaaagtcacagaagtgaaaggatacacgaaaggaggacctggacatgaggaaccaatcccaatggcaacctatggatggaacctagtaaagctatactccgggaaagatgtattctttacaccacctgagaaatgtgacaccctcttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtagaaactttggagcaaatgcaaagaaaacatggaggaatgctagtgcgaaatccactctcaagaaactccactcatgaaatgtactgggtttcatgtggaacaggaaacattgtgtcagcagtaaacatgacatctagaatgttgctaaatcgattcacaatggctcacaggaagccaacatatgaaagagacgtggacttaggcgctggaacaagacatgtggcagtagaaccagaggtggccaacctagatatcattggccagaggatagagaatataaaaaatggacacaaatcaacatggcactatgatgaggacaatccatacaaaacatgggcctatcatggatcatatgaggtcaagccatcaggatcagcctcatccatggtcaatggtgtggtgagactgctaaccaaaccatgggatgtcattcccatggtcacacaaatagccatgactgacaccacaccctttggacaacagagggtgtttaaagagaaagttgacacgcgtacaccaaaagcgaaacgaggcacagcacaaattatggaggtgacagccaggtggttatggggttttctctctagaaacaaaaaacccagaatctgcacaagagaggagttcacaagaaaagtcaggtcaaacgcagctattggagcagtgttcgttgatgaaaatcaatggaactcagcaaaagaggcagtggaagatgaacggttctgggaccttgtgcacagagagagggagcttcataaacaaggaaaatgtgccacgtgtgtctacaacatgatgggaaagagagagaaaaaattaggagagttcggaaaggcaaaaggaagtcgcgcaatatggtacatgtggttgggagcgcgctttttagagtttgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctattggccacgtcaatctttaagctaacctaccaaaacaaggtagtaagggtgcagagaccagcgaaaaatggaaccgtgatggatgtcatatccagacgtgaccagagaggaagtggacaggttggaacctatggcttaaacaccttcaccaacatggaggcccaactaataagacaaatggagtctgagggaatcttttcacccagcgaattggaaaccccaaatctagccgaaagagtcctcgactggttgaaaaaacatggcaccgagaggctgaaaagaatggcaatcagtggagatgactgtgtggtgaaaccaatcgatgacagatttgcaacagccttaacagctttgaatgacatgggaaaggtaagaaaagacataccgcaatgggaaccttcaaaaggatggaatgattggcaacaagtgcctttctgttcacaccatttccaccagctgattatgaaggatgggagggagatagtggtgccatgccgcaaccaagatgaacttgtaggtagggccagagtatcacaaggcgccggatggagcttgagagaaactgcatgcctaggcaagtcatatgcacaaatgtggcagctgatgtacttccacaggagagacttgagattagcggctaatgctatctgttcagccgttccagttgattgggtcccaaccagccgtaccacctggtcgatccatgcccaccatcaatggatgacaacagaagacatgttgtcagtgtggaatagggtttggatagaggaaaacccatggatggaggacaagactcatgtgtccagttgggaagacgttccatacctaggaaaaagggaagatcgatggtgtggatccctaataggcttaacagcacgagccacctgggccaccaacatacaagtggccataaaccaagtgagaaggctcattgggaatgagaattatctagacttcatgacatcaatgaagagattcaaaaacgagagtgatcccgaaggggcactctggtaagccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 21 - DNA, 10723 bp, Dengue virus type 2
agttgttagtctacgtggaccgacaaagacagattctttgagggagctaagctcaacgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggatggcgtgaacatgtgtaccctcatggccatggaccttggtgaattgtgtgaagacacaatcacgtacaagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgattttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccattcggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagagggcatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgctcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgagcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgttcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcggaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagctggagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 22 - DNA, 10707 bp, Dengue virus type 3
agttgttagtctacgtggaccgacaagaacagtttcgactcggaagcttgcttaacgtagtgctaacagttttttattagagagcagatctctgatgaacaaccaacggaagaagacgggaaaaccgtctatcaatatgctgaaacgcgtgagaaaccgtgtgtcaactggaccacagttggcgaagagattctcaaaaggactgctgaacggccagggaccaatgaaattggttatggcgttcatagctttcctcagatttctagccattccaccaacagcaggagtcttggctagatggggaaccttcaagaagtcgggagccattaaggtcctgaaaggcttcaagaaggagatctcaaacatgctgagcataatcaacaaacggaaaaagacatcgctctgtctcatgatgatattgccagcagcacttgctttccacttgacttcacgagatggagagccgcgcatgattgtggggaagaatgaaagaggaaaatccctactttttaagacagcctctggaattaacatgtgcacactcatagccatggacttgggagagatgtgtgatgacacggtcacttacaaatgcccccacattaccgaagtggagcctgaagacattgactgctggtgcaaccttacatcaacatgggtgacttatggaacgtgcaatcaagctggagagcatagacgcgacaaaagatcagtggcgttagctcctcatgtcggcatgggactggacacacgcacccaaacctggatgtcggctgaaggagcttggagacaagtcgagaaggtagagacatgggccctcaggcacccagggttcaccatactagccctatttcttgcccattacataggcacttccttgacccagaaggtggttatttttatactactaatgctggtcaccccatccatgacaatgagatgtgtgggaataggaaacagagattttgtggaaggtctatcaggagctacgtgggttgacgtggtgctcgagcacggggggtgtgtgactaccatggctaagaacaagcccacgctggatatagagcttcagaagaccgaggccacccaactggcgaccctaaggaagctatgcattgaggggaaaattaccaacataacaactgactcaagatgtcctacccaaggggaagcggttttgcctgaggagcaggaccagaactacgtgtgtaagcatacatacgtagacagaggctgggggaacggttgtggcttgtttggcaagggaagcttggtaacgtgtgcgaaatttcaatgcctggaaccaatagagggaaaagtggtgcaatatgagaacctcaaatacaccgtcatcattacagtgcacacaggagaccaacaccaggtaggaaatgaaacgcagggagtcacggctgagataacacctcaggcatcaaccactgaagccatcttgcctgaatatggaacccttgggctagaatgctcaccacggacaggtttggatttcaatgaaatgatcttactaacaatgaagaacaaagcatggatggtacatagacaatggttttttgacctacctctaccatggacatcaggagctacaacagaaacgccaacctggaacaggaaggagcttcttgtgacattcaaaaacgcacatgcgaaaaaacaagaagtagtcgtccttggatcgcaagagggagcaatgcataccgcactgacaggagccacagaaatccaaaactcaggaggcacaagcatttttgcggggcacttaaaatgtagacttaagatggacaaattggaactcaaggggatgagctatgcaatgtgcacgaatacctttgtgttgaagaaagaagtctcagaaacgcagcatgggacaatactcattaaggtcgagtacaaaggggaagatgcaccttgcaagattcctttctccacagaggatggacaagggaaagctcacaatggcagactgatcacagccaacccagtggtgactaagaaggaggagcctgtcaatattgaggctgaacctccttttggggaaagtaatatagtaattggaattggagacaacgccttgaaaatcaactggtacaagaagggaagctctattgggaagatgttcgaggccactgccagaggtgcaaggcgcatggccatcttgggagacacagcttgggactttggatcagtgggtggtgttctgaactcattaggcaaaatggtgcaccaaatattcggaagtgcttacacagccctattcagtggagtctcttgggtgatgaaaattggaataggtgttctcttgacttggatagggttgaattcaaaaaacacatccatgtcattttcatgcattgcgataggaatcattacactctatctgggagctgtggtacaagctgacatggggtgtgtcataaactggaaaggcaaagaactcaaatgtggaagtggaattttcgtcaccaacgaggtccatacctggacagagcaatacaaattccaagcagactccccaaaaagattggcgacagccattgcaggcgcttgggagaatggagtgtgcggaattaggtcaacaaccagaatggagaatctcttgtggaagcaaatagccaatgaactgaactacatattatgggaaaacaatatcaaattaacggtagttgtgggcgatacaattggggtcttagagcaaggaaaaagaacactaacaccacaacccatggagctaaaatactcatggaaaacatggggaaaggcaaaaatagtgacagctgaaacacaaaattcctccttcataatagacgggccaaacacaccggagtgtccaagtgcctcaagagcatggaatgtgtgggaggtggaagattacgggttcggagtcttcacaaccaacatatggctgaaactccgagatgtgtacacccaactatgtgaccataggctaatgtcggcagccgtcaaggatgagagggccgtacacgccgacatgggctattggatagaaagccaaaagaatggaagttggaagctagaaaaagcatccctcatagaggtgaaaacctgcacatggccaaaatcacacactctttggagcaatggtgtgctagagagtgacatgatcatcccaaagagtctagctggtcctatttcgcaacacaactacaggcctgggtaccacacccaaacagcaggaccctggcacttaggaaaattggagctggacttcaactattgtgaaggaacaacagttgtcatcacagaaaactgtgggacaagaggcccatcattgagaacaacaacagtgtcagggaagttgatacacgaatggtgttgccgctcgtgcacacttcctcccctgcgatacatgggagaagacggctgctggtatggcatggaaatcagacccatcagtgagaaagaagagaacatggtaaagtctttagtctcagcgggaagtggaaaggtggacaacttcacaatgggtgtcttgtgtttggcaatcctctttgaagaggtgatgagaggaaaatttgggaagaaacacatgattgcaggggttctcttcacgtttgtgctccttctctcagggcaaataacatggagagacatggcgcacacactaataatgattgggtccaacgcctctgacaggatgggaatgggcgtcacctacctagctttaattgcaacatttaaaatccagccattcttggctttgggatttttcctaagaaaactgacatctagagaaaatttattgttaggagttgggctggctatggcaacaacgttacaactgccagaggacattgaacaaatggcaaatggaatcgctctggggctcatggctcttaaactgataacacaatttgaaacataccaattatggacggcattagtctccttaacgtgttcaaatacaattcttacgttgactgttgcctggagaacagccaccctgattttggccggagtttcgcttttaccagtgtgccagtcttcgagcatgaggaaaacagactggcttccaatgacagtggcagctatgggagttccacccctaccactttttatttttagcttgaaagacacactcaaaaggagaagctggccactgaatgaaggggtgatggctgttgggcttgtgagcattctggccagttctctccttagaaatgatgtgcccatggctggaccattagtggccgggggcttgctgatagcgtgctacgtcataactggcacgtcagcagacctcactgtggaaaaagcagcagatgtaacatgggaggaagaggctgagcaaacaggagtgtcccacaacttaatgatcacagttgatgatgatggaacaatgagaataaaagatgatgagactgagaacatcctaacagtgcttttaaaaacagcattactaatagtatcaggcatctttccatactccatacccgcaacattgttggtctggcatacttggcaaaagcaaacccaaaggtccggcgttctgtgggacgtacccagccccccagagacacagaaagcagaactggaagaaggggtttataggatcaaacagcaaggaattcttgggaaaacccaagtaggggttggagtacagaaagaaggagtcttccacaccatgtggcacgtcacaagaggggcagtgttgacacataatgggaaaagactggaaccaaactgggctagcgtgaaaaaagatctgatttcatacggaggaggatggagattgagcgcgcaatggcaaaagggggaggaggtgcaggttattgccgtggagcctgggaagaacccaaagaactttcaaaccatgccaggcacttttcagactacaacaggggaaataggagcaattgcactggatttcaagcctggaacttcaggatctcctatcataaacagagagggaaaggtagtgggactgtatggcaatggagtggttacaaagaatggtggctacgtcagcggaatagcgcaaacaaatgcagaaccagatggaccgacaccagagttggaagaagagatgttcaaaaagcgaaatctaaccataatggatcttcatcctgggtcaggaaagacacggaaataccttccagctattgttagagaggcaatcaagagacgtttaagaactctaattttggcaccgacaagggtggttgcagctgagatggaagaagcattgaaagggctcccaataaggtaccaaacaacagcaacaaaatctgaacacacaggaagagagattgttgatctaatgtgccacgcaacgttcacaatgcgcttgctgtcaccagttagggttccaaattataacttgataataatggatgaggcccatttcacagacccagccagcatagcggctagagggtacatatcaactcgtgttggaatgggagaggcagccgcaattttcatgacagcaacgccccctggaacagctgatgcctttcctcagagcaacgctccaattcaagatgaagaaagggacataccagaacgctcatggaattcaggcaatgaatggattaccgacttcgctgggaaaacggtgtggtttgtccccagcattaaagccggaaatgacatagcaaactgcttgcggaaaaacgggaaaaaggtcattcaacttagtaggaagacttttgacacagaatatcagaagactaaactgaatgattgggacttcgtggtgacaactgacatttcagaaatgggggccaatttcaaagcagatagagtgatcgacccaagaagatgtctcaaaccagtgatcctgacagatggaccagagcgggtgatcctggctggaccaatgccagtcaccgcggcgagtgctgcgcaaaggagaggaagagttggcaggaacccacaaaaagaaaatgaccagtacatattcacgggccagcctctcaacaatgatgaagaccatgctcactggacagaagcaaaaatgctgctggacaacattaacacaccagaagggattataccagctctctttgaaccagaaagggagaagtcagccgccatagacggtgagtatcgcctgaagggtgagtccaggaagactttcgtggaactcatgaggaggggtgaccttccagtttggttagcccataaagtagcatcagaagggatcaaatatacagatagaaaatggtgctttgatggacaacgcaataatcaaattttagaggagaacatggatgtggaaatctggacaaaggaaggagaaaagaaaaaattgagacctaggtggcttgatgcccgcacttattcagatcccttagcactcaaggaatttaaggactttgcggctggcagaaagtcaatcgcccttgatcttgtgacagaaataggaagagtgccttcacacctagcccacagaacgagaaacgctctggacaatctggtgatgctgcatacgtcagaacatggcggtagggcctacaggcatgcggtggaggaactaccagaaacaatggaaacactcttactcttgggactcatgatcttgttgacaggtggagcaatgcttttcttgatatcaggaaaagggattggaaagacttcaataggactcatttgtgtaattgcctccagcggcatgttgtggatggccgaaatcccactccagtggatcgcgtcggctatagtcctggagttttttatgatggtgttgcttataccagaaccagaaaagcagagaaccccccaagacaaccaactcgcatatgtcgtgataggcatacttacattggctgcaataatagcagccaatgaaatgggactgttggaaactacaaagagagatttaggaatgtctaaggagccaggtgttgtttctccaaccagctatttggatgtggacttgcacccagcatcagcctggacattgtacgccgtggccactacagtaataacaccaatgttaagacataccatagagaattctacagcaaatgtgtccctggcagctatagccaaccaggcagtggtcctgatgggtttggacaaaggatggccaatatcaaaaatggacttaggcgtaccactactggcattgggttgctattcacaagtgaacccactgactctaacagcggcagtacttttgctaatcacacattatgctattataggtccaggattgcaggcaaaagccactcgtgaagctcagaaaaggacagctgctggaataatgaagaatccaacggtggatgggataatgacaatagacctagatcctgtaatatatgattcaaaatttgaaaagcaactgggacaggttatgctcctggttttgtgtgcagttcaacttttgttaatgagaacatcatgggccttgtgtgaagctttaactctagctacaggaccaataacaacactctgggaaggatcacctggaaagttttggaacaccacgatagctgtttccatggcgaacatttttagagggagctatttagcaggagctgggcttgctttttctattatgaaatcagttggaacaggaaaaagaggaacaggttcacaaggcgaaactttaggagaaaaatggaaaaagaaattaaatcaattatcccggaaagagtttgacctttacaagaaatctggaatcactgaagtggatagaacagaagccaaagaagggttgaaaagaggagaaataacacatcatgccgtgtccagaggtagcgcaaaacttcaatggtttgtggagagaaacatggtcattcccgaaggaagagtcatagacttgggctgtggaagaggaggctggtcatactactgtgcaggactgaaaaaagtcacagaagtgcgaggatacacaaaaggcggtccaggacacgaagaaccagtacctatgtctacatatggatggaacatagttaagttaatgagtggaaaggatgtgttttatcttccacctgaaaagtgtgataccctgttgtgtgacatcggagaatcttcaccaagcccaacagtggaagaaagcagaactataagagttttgaagatggttgaaccatggctaaaaaacaaccagttttgcattaaagtattgaacccttacatgccaactgtgattgagcacctagaaagactacaaaggaaacatggaggaatgcttgtgagaaatccactttcacgaaactccacgcacgaaatgtactggatatctaatggcacaggtaacattgtctcttcagtcaacatggtatctagattgctactgaacaggttcacgatgacacacaggagacctaccatagagaaagatgtggatttaggagcaggaactcgacatgttaatgcggaaccagaaacacccaacatggatgtcattggggaaagaataaaaaggatcaaggaggagcacaattcaacatggcactatgatgacgaaaacccctacaaaacgtgggcttaccatggatcctatgaagtcaaagccacaggctcagcctcctccatgataaatggagtcgtgaaactcctcactaaaccatgggatgtggtgcccatggtgacacagatggcaatgacagatacaactccatttggccagcagagagtctttaaagagaaagtggacaccaggacacccaggcccatgccagggacaagaaaggttatggggatcacagcggagtggctctggagaaccctgggaaggaacaaaagacccaggttatgcacaagggaagagtttacaaaaaaggtcagaactaacgcagccatgggcgccgttttcacagaggagaaccaatgggacagtgcgaaagctgctgttgaggatgaagaattttggaaacttgtggacagagaacgtgaactccacaaattgggcaagtgtggaagctgtgtttacaacatgatgggcaagagagagaagaaacttggagagtttggcaaagcaaaaggcagtagagctatatggtacatgtggttgggagccaggtaccttgagttcgaagcccttggattcctaaatgaagaccactggttctcgcgtgacaactcttacagtggagtagaaggagaaggactgcacaagctaggctacatattaagggacatttccaagatacccggaggagctatgtatgctgatgacacagctggttgggacacaagaataacagaagatgacctgcacaatgaggaaaagatcacacagcaaatggaccctgaacacaggcagttagcgaacgctatatttaagctcacataccaaaacaaagtggtcaaagttcaacgaccgactccaacgggcacggtaatggacatcatatctaggaaagaccaaagaggcagtggacaggtgggaacttatggtctgaatacattcaccaacatggaagtccagttagtcagacaaatggaaggagaaggtgtgctgtcaaaggcagacctcgagaaccctcatctgccagagaagaaaattacacaatggttggaaaccaaaggagtggagaggttaaaaagaatggccattagcggggatgattgtgtagtgaaaccaatcgatgacaggttcgctaatgccctgcttgctctgaacgatatgggaaaggttcggaaagacatacctcaatggcagccatcaaagggatggcatgattggcaacaggttcctttctgctcccaccactttcatgaattgatcatgaaagatggaagaaagttagtggttccctgtagaccccaggacgaactaataggaagagcaagaatctctcaaggagcgggatggagccttagagagaccgcatgtctggggaaagcctacgctcaaatgtggagtctcatgtactttcacagaagagatctcagactagcatccaacgccatatgttcagcagtaccagtccactgggtccccacaagtagaacgacatggtctattcatgctcaccatcagtggatgactacagaagacatgcttactgtctggaacagggtgtggatcgaggacaatccatggatggaagacaaaactccagttacaacctgggaaaatgttccatatctagggaagagagaagaccaatggtgcggatcacttattggtctcacctccagagcaacctgggcccagaacatacccacagcaattcaacaggtgagaagccttataggcaatgaagagtttctggattacatgccttcaatgaagagattcaggaaggaggaggagtcggaaggagccatttggtaaacgtaggaagtgaaaaagaggcaaactgtcaggccaccttaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaacaaaaacagcatattgacgctgggagagaccagagatcctgctgtctcctcagcatcattccaggcacagaacgccagaaaatggaatggtgctgttgaatcaacaggttct

SEQ ID NO: 23 - DNA, 10650 bp, Dengue virus type 4
agttgttagtctgtgtggaccgacaaggacagttccaaatcggaagcttgcttaacacagttctaacagtttgtttaaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgggtcctttccatcccaccaacagcagggattctgaagagatggggacagttgaagaaaaataaggccatcaagatactgattggatttaggaaggagataggccgcatgctgaacattttgaacgggagaaaaaggtcaacgataacattgctgtgcttgattcccaccgtaatggcgtttcacttgtcaacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctgatgatgctggtcgccccatcctacggaatgcgatgcgtaggagtgggaaacagagactttgtggaaggagtctcaggtggagcatgggtcgacctggtgctagaacatggaggatgcgtcacaaccatggcccagggaaaaccaaccttggattttgaactgactaagacaacagccaaggaagtggctctgttaagaacctattgcattgaagcctcaatatcaaacataactacggcaacaagatgtccaacgcaaggagagccgtatctgaaagaggaacaggaccaacagtacatttgccggagagatgtggtagacagagggtggggcaatggctgtggcttgtttggaaaaggaggagttgtgacatgtgcgaagttttcatgttcggggaagataacaggcaatctggtccaaattgagaaccttgagtacacagtggttgtaacagtccacaatggagacacccatgcagtaggaaatgacacatccaatcatggagttacagccacgataactcccaggtcaccatcggtggaagtcaaattgccggactatggagaactaacactcgattgtgaacccaggtctggaattgactttaatgagatgatcctaatgaaaatgaaaaagaaaacatggctcgtgcacaagcaatggtttttggatctgcctcttccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatctcaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgcgttatctcatccacccctttggctgagaataccaacagtgtaaccaacatagaattagaacccccctttggggacagctacatagtgataggtgttggaaacagcgcattaacactccattggttcaggaaagggagctccattggcaagatgtttgagtccacatacagaggtgcaaaacgaatggccattctgggtgaaacagcttgggattttggttccgttggtggactgttcacatcattgggaaaggctgtgcaccaggtttttggaagtgtgtatacaaccatgtttggaggagtctcatggatgattagaatcctaattgggttcttagtgttgtggattggcacgaactcaaggaacacttcaatggccatgacgtgcatagctgttggaggaatcactctgtttctgggcttcacagttcaagcggacatgggttgtgtggtgtcatggagtgggagagaattgaagtgtggaagcggaatttttgtggttgacaacgtgcacacttggacagaacagtacaaattccaaccagagtccccagcgagactagcgtctgcaatattaaatgcccacaaagatggggtctgtggaattagatcaaccacgaggctggaaaatgtcatgtggaagcaaataaccaatgagctaaactatgttctctgggaaggaggacatgatctcactgtagtggctggggacgtgaagggagtgttgaccaaaggcaagagagcactcacacccccagtgagtgatctgaaatattcatggaagacatggggaaaagcaaaaatcttcaccccagaagcaagaaacagcacatttttgatagatggaccagacacctctgaatgccccaatgaacgaagagcatggaactctcttgaggtggaagattatggatttggcatgttcacgacgaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacccaaacagtgggcccatggcacttaggcaaattagagatagactttggagaatgccccggaacaacagtcacaattcaggaggattgtgaccatagaggcccatctttgaggaccaccactgcatctggaaaactagtcacgcaatggtgttgccgctcctgcacgatgcctcccttaaggttcttgggagaagatgggtgctggtatgggatggagattaggcccttgagtgaaaaagaagagaacatggtcaaatcacaggtgacggccggacagggcacgtcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacctactacgagccctcatcatgttgggggacactatgtctggtagaataggaggacagatccacctagccatcatggcagtgttcaagatgtcaccaggatacgtgctgggtgtgttcttaaggaaactcacttcaagagagacagcactaatggtaataggaatggccatgacaacggtactttcaattccacatgaccttatggaactcattgatggaatatcactaggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttagggagcgctcttttaaagaatgatgtccctttggctggcccaatggtggcaggaggcttacttctggcggcttacgtgatgagtggcagctcagcagatctgtcactagagaaggccgccaatgtgcagtgggatgaaatggcagacataacaggctcaagcccaatcatagaagtgaagcaagatgaagatggctctttctccatacgggacgtcgaggaaaccaatatgataacccttttggtgaaactggcactgataacagtgtcaggtctctaccccttggcaattccaatcacaatgaccttatggtacatgtggcaagtgaaaacacaaagatcaggagccctgtgggacgtcccctcacccgctgccactcaaaaagccgcactgtctgaaggagtgtacaggatcatgcaaagagggttattcgggaaaactcaggttggagtagggatacacatggaaggtgtatttcatacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgctatagaaccaggaaaaaatcccaaacatgtccaaacgaaacctggccttttcaagaccctaactggagaaattggagcagtaacattggatttcaaacccggaacgtctggttctcccatcatcaacaggaaaggaaaagtcatcggactctatggaaatggagtagttaccaaatcaggtgactacgtcagtgccataacgcaagccgaaagaattggagagccagattatgaagtggatgaggatatttttcgaaagaaaagattaactataatggacttacaccccggagctggaaagacaaaaagaattcttccatcaatagtgagagaagccttaaaaaggaggctgcgaactttgattttagctcccacgagagtggtggcggccgagatggaagaggccctacgtggactgccaatccgttatcagaccccagctgtgaaatcagaacacacaggaagagagattgtagacctcatgtgtcacgcaaccttcacaacaagacttttgtcatcaaccagggttccaaattacaaccttatagtgatggatgaagcacatttcaccgatccttccagtgtcgcggctagaggatacatttcgaccagggtggaaatgggagaggcagcagccatcttcatgaccgcaaccccaccaggtgcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagtcatccagttgagcaggaaaacctttggtacagagtatccaaaaacgaaactcacggactgggactttgtggtcactacagacatatctgaaatgggggctaattttagagctgggagagtgatagaccctagaagatgcctcaagccagttatcctaacagatgggccagagagagtcattttagcaggtcctattccagtgactccagcaagcgctgctcagagaagagggcgagtaggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacaaagatcgggaatggtgcttcacaggggaaagaaataaccaaattttagaagaaaacatggaggttgaaatttggactagagagggagaaaagaaaaaattgaggccaagatggttagatgcacgtgtatacgctgaccccatggctttgaaggatttcaaggagtttgccagtggaaggaagagtataactctagacatcctaacagagattgccagtttgccagcatacctttcctctagggccaagctcgcccttgataacatagtcatgctccacacaacagaaagaggagggagggcctatcaacacgccctgaacgaacttccggagtcactggaaacactcatgcttgtagccttactaggtgctatgacagcaggtatcttcctgtttttcatgcaagggaaaggaatagggaaattgtcaatgggtttgataaccattgcggtggctagtggcttgctctggatagcagaaattcaaccccagtggatagcggcctcaatcatactagagttttttctcatggtactgttgataccagaaccagaaaaacaaaggaccccacaagacaatcaattgatctatgtcatattgaccgttctcaccatcattggtctgatagcagccaacgagatggggctgattgaaaaaacaaaaacggactttgggttctaccaggtaaaaacagaaaccaccatcctcgatgtggacttgagaccagcttcagcatggacgctctatgcagtagccaccacagttttgactcccatgctgagacacaccatagaaaacacgtcggccaacctatctctagcagccatcgccaaccaggcagccgtcctaatggggcttggaaagggatggccgctacacagaatggacctcggtgtgccgctgttagcaatgggatgctattctcaagtgaacccaacaaccttgacagcatccttagtcatgctttttgtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcctgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaactcctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtttctagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggctggaatttggtcaaactccattcaggggttgacgtgttttacaaacccacagagcaagtggacaccctgctctgtgatattggggagtcatcttctaatccaacaatagaggagggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctctactgaaacagaaaaaccagacatgacaatcattgggagaaggcttcagcgattgcaagaagagcacaaagaaacatggcattatgatcaggaaaacccatacagaacctgggcgtatcatggaagctatgaagctccttcgacaggctctgcatcctccatggtgaacggggtggtgaaactgctaacaaaaccctgggatgtgattccaatggtgactcagttagccatgacagatacaaccccttttgggcaacaaagagtgttcaaagagaaggtggataccagaacaccacaaccaaaaccaggcacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgactccagattttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgagaggagcggtgatggatatcatatccaggaaagaccaaagaggtagtggacaagttggaacatatggcttgaacacattcaccaacatggaagttcaactcatccgccaaatggaagctgaaggagtcatcacacaagatgacatgcagaacccaaaagggttgaaagaaagagttgagaaatggctgagagagtgtggtgtcgacaggttaaagaggatggcaatcagtggggatgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggggctggatggagcttaagagaaacagcctgcctgggcaaagcttacgcccagatgtggtcgcttatgtacttccacagaagggatctgcgtttagcctccatggccatatgctcagcagtcccaacggaatggtttccaacaagcagaacaacatggtcaatccacgctcatcaccagtggatgaccactgaagacatgctcaaagtgtggaacagagtgtggatagaagacaaccctaatatgactgacaagactccagtccattcgtgggaagatataccttacctagggaaaagagaggatttgtggtgtggatccctgattggactttcttctagagccacctgggcgaagaacattcacacggccataactcaggtcaggaatctgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaacaatgggaggcgtaataatccccagggaggccatgcgccacggaagctgtacgcgtggcatattggactagcggttagaggagacccctcccatcactgacaaaacgcagcaaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 24 - DNA, 10969 bp, Japanese encephalitis virus
agaagtttatctgtgtgaacttcttggcttagtatcgttgagaagaatcgagagattagtgcagtttaaacagttttttagaacggaagataaccatgactaaaaaaccaggagggcccggtaaaaaccgggctatcaatatgctgaaacgcggcctaccccgcgtattcccactagtgggagtgaagagggtagtaatgagcttgttggacggcagagggccagtacgtttcgtgctggctcttatcacgttcttcaagtttacagcattagccccgaccaaggcgcttttaggccgatggaaagcagtggaaaagagtgtggcaatgaaacatcttactagtttcaaacgagaacttggaacactcattgacgccgtgaacaagcggggcagaaagcaaaacaaaagaggaggaaatgaaggctcaatcatgtggctcgcgagcttggcagttgtcatagcttgtgcaggagccatgaagttgtcgaatttccaggggaagcttttgatgaccatcaacaacacggacattgcagacgttatcgtgattcccacctcaaaaggagagaacagatgctgggtccgggcaatcgacgtcggctacatgtgtgaggacactatcacgtacgaatgtcctaagcttaccatgggcaatgatccagaggatgtggattgctggtgtgacaaccaagaagtctacgtccaatatggacggtgcacgcggaccaggcattccaagcgaagcaggagatccgtgtcggtccaaacacatggggagagttcactagtgaataaaaaagaggcttggctggattcaacgaaagccacacgatatctcatgaaaactgagaactggatcataaggaatcctggctatgctttcctggcggcggtacttggctggatgcttggcagtaacaacggtcaacgcgtggtatttaccatcctcctgctgttggtcgctccggcttacagttttaattgtctgggaatgggcaatcgtgacttcatagaaggagccagtggagccacttgggtggacttggtgctagaaggagatagctgcttgacaatcatggcaaacgacaaaccaacattggacgtccgcatgattaacatcgaagctagccaacttgctgaggtcagaagttactgctatcatgcttcagtcactgacatctcgacggtggctcggtgccccacgactggagaagcccacaacgagaagcgagctgatagtagctatgtgtgcaaacaaggcttcactgaccgtgggtggggcaacggatgtggacttttcgggaagggaagcattgacacatgtgcaaaattctcctgcaccagtaaagcgattgggagaacaatccagccagaaaacatcaaatacgaagttggcatttttgtgcatggaaccaccacttcggaaaaccatgggaattattcagcgcaagttggggcgtcccaggcggcaaagtttacagtaacacccaatgctccttcgataaccctcaaacttggtgactacggagaagtcacactggactgtgagccaaggagtggactgaacactgaagcgttttacgtcatgaccgtggggtcaaagtcatttctggtccatagggagtggtttcatgacctcgctctcccctggacgtccccttcgagcacagcgtggagaaacagagaactcctcatggaatttgaaggggcgcacgccacaaaacagtccgttgttgctcttgggtcacaggaaggaggcctccatcaggcgttggcaggagccatcgtggtggagtactcaagctcagtgaagttaacatcaggccacctgaaatgtaggctgaaaatggacaaactggctctgaaaggcacaacctatggcatgtgtacagaaaaattctcgttcgcgaaaaatccggtggacactggtcacggaacagttgtcattgaactctcctactctgggagtgatggcccctgcaaaattccgattgtttccgttgcgagcctcaatgacatgacccccgttgggcggctggtgacagtgaaccccttcgtcgcgacttccagtgccaactcaaaggtgctggtcgagatggaaccccccttcggagactcctacatcgtagttggaaggggagacaagcagatcaaccaccattggcacaaagctggaagcacgctgggcaaggccttttcaacaactttgaagggagctcaaagactggcagcgttgggcgacacagcctgggactttggctctattggaggggtcttcaactccataggaagagccgttcaccaagtgtttggtggtgccttcagaacactctttgggggaatgtcttggatcacacaagggctaatgggtgccctactgctctggatgggcgtcaacgcacgagaccgatcaattgctttggccttcttagccacaggaggtgtgctcgtgttcttagcgaccaatgtgcatgctgacactggatgtgccattgacatcacaagaaaagagatgagatgtggaagtggcatcttcgtgcacaacgacgtggaagcctgggtggataggtataaatatttgccagaaacgcccagatccctagcgaagatcgtccacaaagcgcacaaggaaggcgtgtgcggagtcagatctgtcactagactggagcaccaaatgtgggaagccgtaagggacgaattgaacgtcctgctcaaagagaatgcagtggacctcagtgtggttgtgaacaagcccgtgggaagatatcgctcagcccctaaacgcctatccatgacgcaagagaagtttgaaatgggctggaaagcatggggaaaaagcatcctctttgccccggaattggctaactccacatttgtcgtagatggacctgagacaaaggaatgccctgatgagcacagagcttggaacagcatgcaaatcgaagacttcggctttggcatcacatcaacccgtgtgtggctgaaaattagagaggagagcactgacgagtgtgatggagcgatcataggcacggctgtcaaaggacatgtggcagtccatagtgacttgtcgtactggattgagagtcgctacaacgacacatggaaacttgagagggcagtctttggagaggtcaaatcttgcacttggccagagacacacaccctttggggagatgatgttgaggaaagtgaactcatcattccgcacaccatagccggaccaaaaagcaagcacaatcggagggaagggtataagacacaaaaccagggaccttgggatgagaatggcatagtcttggactttgattattgcccagggacaaaagtcaccattacagaggattgtagcaagagaggcccttcggtcagaaccactactgacagtggaaagttgatcactgactggtgctgtcgcagttgctcccttccgcccctacgattccggacagaaaatggctgctggtacggaatggaaatcagacctgttatgcatgatgaaacaacactcgtcagatcacaggttgatgctttcaaaggtgaaatggttgacccttttcagctgggccttctggtgatgtttctggccacccaggaagtccttcgcaagaggtggacggccagattgaccattcctgcggttttgggggtcctacttgtgctgatgcttgggggtatcacttacactgatttggcgaggtatgtggtgctagtcgctgctgctttcgcagaggccaacagtggaggagacgtcctgcaccttgctttgattgccgtttttaagatccaaccagcatttctagtgatgaacatgcttagcacgagatggacgaaccaagaaaacgtgattctggtcctaggggctgcctttttccaattggcctcagtagatctgcaaataggagtccacggaatcctgaatgccgccgctatagcatggatgattgtccgagcgatcaccttccccacaacctcctccgtcaccatgccagtcttagcgcttctaactccggggatgagggctctatacctagacacttacagaatcatcctcctcgtcatagggatttgctccctgctgcacgagaggaaaaagaccatggcaaaaaagaaaggagctgtactcttgggcttagcgctcacatccactggatggttctcgcccaccactatagctgccggactaatggtctgcaacccaaacaagaagagagggtggccagctactgagtttttgtcggcagttggattgatgtttgccatcgtaggtggtttggccgagttggatattgaatccatgtcaatacccttcatgctggcaggtctcatggcagtgtcctacgtggtgtcaggaaaagcaacagatatgtggcttgaacgggccgccgacatcagctgggagatggatgctgcaatcacaggaagcagtcggaggctggatgtgaaactggatgatgacggagattttcacttgattgatgatcccggtgttccatggaaggtctgggtcctgcgcatgtcttgcattggcttagccgccctcacgccttgggccatcgttcccgccgctttcggttattggctcactttaaaaacaacaaaaagagggggcgtgttttgggacacgccatccccaaaaccttgctcaaaaggagacaccactacaggagtctaccgaattatggctagagggattcttggcacttaccaggccggcgtcggagtcatgtacgagaatgttttccacacactatggcacacaactagaggagcagccattatgagtggagaaggaaaattgacgccatactggggtagtgtgagagaagaccgcatagcttacggaggcccatggaggtttgaccgaaaatggaatggaacagatgacgtgcaagtgatcgtggtagaaccggggaaggctgcagtaaacatccagacaaaaccaggagtgtttcggactcccttcggggaggttggggctgttagtctggattacccgcgaggaacatccggctcacccattctggattccaatggagacattataggcctatacggcaatggagttgagcttggcgatggctcatacgtcagcgccatcgtgcagggtgaccgtcaggaggaaccagtcccagaagcttacaccccaaacatgttgagaaagagacagatgactgtgctagatttgcaccctggttcagggaaaaccaggaaaattctgccacaaataattaaggacgctatccagcagcgcctaagaacagctgtgttggcaccgacgcgggtggtagcagcagaaatggcagaagctttgagagggctcccagtacgatatcaaacttcagcagtgcagagagagcaccaagggaatgaaatagtggatgtgatgtgccacgccactctgacccatagactgatgtcaccgaacagagtgcccaactacaacctatttgtcatggatgaagctcatttcaccgacccagccagtatagccgcacgaggatacattgctaccaaggtggaattaggggaggcagcagccatctttatgacagcgaccccgcctggaaccacggatccttttcctgactcaaatgccccaatccatgatttgcaagatgagataccagacagggcatggagcagtggatacgaatggatcacagaatatgcgggtaaaaccgtgtggtttgtggcgagcgtaaaaatggggaatgagattgcaatgtgcctccaaagagcggggaaaaaggtcatccaactcaaccgcaagtcctatgacacagaatacccaaaatgtaagaatggagactgggattttgtcattaccaccgacatctctgaaatgggggccaacttcggtgcgagcagggtcatcgactgtagaaagagcgtgaaacccaccatcttagaagagggagaaggcagagtcatcctcggaaacccatctcccataaccagtgcaagcgcagctcaacggaggggcagagtaggcagaaaccccaaccaagttggagatgaataccactatgggggggctaccagtgaagatgacagtaacctagcccattggacagaggcaaagatcatgttagacaacatacacatgcccaatggactggtggcccagctctatggaccagagagggaaaaggctttcacaatggatggcgaataccgtctcagaggtgaagaaaagaaaaacttcttagagctgcttaggacggctgacctcccggtgtggctggcctacaaggtggcgtccaatggcattcagtacaccgacagaaagtggtgttttgatgggccgcgtacgaatgccatactggaggacaacaccgaggtagagatagtcacccggatgggtgagaggaaaatcctcaagccgagatggcttgatgcaagagtttatgcagatcaccaagccctcaagtggttcaaagactttgcagcagggaagagatcagccgttagcttcatagaggtgctcggtcgcatgcctgagcatttcatgggaaagacgcgggaagctttagacaccatgtacttggttgcaacggctgagaaaggtgggaaagcacaccgaatggctctcgaagagctgccagatgcactggaaaccatcacacttattgtcgccattactgtgatgacaggaggattcttcctactaatgatgcagcgaaagggtatagggaagatgggtcttggagctctagtgctcacgctagctaccttcttcctgtgggcggcagaggttcctggaaccaaaatagcagggaccctgctgatcgccctgctgctgatggtggttctcatcccagaaccggaaaaacagaggtcacagacagataaccaactggcggtgtttctcatctgtgtcttgaccgtggttggagtggtggcagcaaacgagtacgggatgctagaaaaaaccaaagcagatctcaagagcatgtttggcggaaagacgcaggcatcaggactgactggattgccaagcatggcactggacctgcgtccagccacagcctgggcactgtatggggggagcacagtcgtgctaacccctcttctgaagcacctgatcacgtcggaatacgtcaccacatcgctagcctcaattaactcacaagctggctcattattcgtcttgccacgaggcgtgccttttaccgacctagacttgaccgttggcctcgtcttccttggctgttggggtcaaatcaccctcacaacgtttctgacagccatggttctggcgacacttcactatgggtacatgctccctggatggcaagcagaagcactcagggctgcccagagaaggacagcggctggaataatgaagaatgccgttgttgacggaatggtcgccactgatgtgcctgaactggaaaggactactcctctgatgcaaaagaaagtcggacaggtgctcctcataggggtaagcgtggcagcgttcctcgtcaaccctaatgtcaccactgtgagagaagcaggggtgttggtgacggcggctacgcttactttgtgggacaatggagccagtgccgtttggaattccaccacagccacgggactctgccatgtcatgcgaggtagctacctggctggaggctccattgcttggactctcatcaagaacgctgataagccctccttgaaaaggggaaggcctgggggcaggacgctaggggagcagtggaaggaaaaactaaatgccatgagcagagaagagttttttaaataccggagagaggccataatcgaggtggaccgcactgaagcacgcagggctagacgtgaaaataacatagtgggaggacatccggtttcgcgaggctcagcaaaactccgttggcttgtggagaaaggatttgtctcgccaataggaaaagtcattgatctagggtgtgggcgtggaggatggagctactacgcagcaaccctgaagaaggtccaggaagtcagaggatacacgaaaggtggggcgggacatgaagaaccgatgctcatgcagagctacggctggaacctggtctccctgaagagtggagtggacgtgttttacaaaccttcagagcccagtgacaccctgttctgtgacataggggaatcctccccaagtccagaagtagaagaacaacgcacactacgcgtcctagagatgacatctgactggttgcaccgaggacctagagagttctgcattaaagttctctgcccttacatgcccaaggttatagaaaaaatggaagttctgcagcgccgcttcggaggtgggctagtgcgtctccccctgtcccgaaactccaatcacgagatgtattgggttagtggagccgctggcaatgtggtgcacgctgtgaacatgaccagccaggtactactggggcgaatggatcgcacagtgtggagagggccaaagtatgaggaagatgtcaacctagggagcggaacaagagccgtgggaaagggagaagtccatagcaatcaggagaaaatcaagaagagaatccagaagcttaaagaagaattcgccacaacgtggcacaaagaccctgagcatccataccgcacttggacataccacggaagctatgaagtgaaggctactggctcagccagctctctcgtcaacggagtggtggagctcatgagcaaaccttgggacgccattgccaacgtcaccaccatggccatgactgacaccaccccttttggacagcaaagagttttcaaggagaaagttgacacgaaggctcctgagccaccagctggagccaaggaagtgctcaacgagaccaccaactggctgtgggcccacttgtcacgggaaaaaagaccccgcttgtgcaccaaggaagaattcataaagaaagtcaacagcaacgcggctcttggagcagtgttcgctgaacagaatcaatggagcacggcgcgtgaggctgtggatgacccgcggttttgggagatggttgatgaagagagggaaaaccatctgcgaggagagtgtcacacatgtatctacaacatgatgggaaaaagagagaagaagcctggagagtttggaaaagctaaaggaagcagggccatttggttcatgtggcttggagcacggtatctagagtttgaagctttggggttcctgaatgaagaccattggctgagccgagagaattcaggaggtggagtggaaggctcaggcgtccaaaagctgggatacatcctccgtgacatagcaggaaagcaaggagggaaaatgtacgctgatgataccgccgggtgggacactagaattaccagaactgatttagaaaatgaagctaaggtactggagctcctagacggtgaacaccgcatgctcgcccgagccataattgaactgacttacaggcacaaagtggtcaaggtcatgagacctgcagcagaaggaaagaccgtgatggacgtgatatcaagagaagatcaaagggggagtggacaggtggtcacttatgctcttaacactttcacgaacatcgctgtccagctcgtcaggctgatggaggctgagggggtcattggaccacaacacttggaacagctacctagaaaaaccaagatagctgtcaggacctggctctttgagaatggagaggagagagtgaccaggatggcgatcagcggagacgactgtgtcgtcaagccgctggacgacagattcgccacagccctccacttcctcaacgcaatgtcaaaggtcagaaaagacatccaggaatggaagccttcgcatggctggcacgattggcagcaagttcccttctgctctaaccattttcaggagattgtgatgaaagatggaaggagtatagttgtcccgtgcagaggacaggatgagctgataggcagggctcgcatctctccaggagctggatggaatgtgaaggacacagcttgcctggccaaagcatatgcacagatgtggctactcctatacttccatcgcagggacttgcgtctcatggcaaatgcgatttgctcagcagtgccagtggattgggtgcccacaggcaggacatcctggtcaatacactcgaaaggagagtggatgaccacggaagacatgctgcaggtctggaacagagtctggattgaagaaaatgaatggatgatggacaagactccaatcacaagctggacagacgttccgtatgtgggaaagcgtgaggacatctggtgtggcagcctcatcggaacgcgatccagagcaacctgggctgagaacatctatgcggcgataaaccaggttagagctgtcattgggaaagaaaattatgttgactacatgacctcactcaggagatacgaagacgtcttgatccaggaagacagggtcatctagtgtgatttaaggtagaaaagtagactatgtaaataatgtaaatgagaaaatgcatgcatatggagtcaggccagcaaaagctgccaccggatactgggtagacggtgctgcctgcgtctcagtcccaggaggactgggttaacaaatctgacaacagaaagtgagaaagccctcagaaccgtctcggaagtaggtccctgctcactggaagttgaaagaccaacgtcaggccacaaatttgtgccactccgctagggagtgcggcctgcgcagccccaggaggactgggttaccaaagccgttgagcccccacggcccaagcctcgtctaggatgcaatagacgaggtgtaaggactagaggttagaggagaccccgtggaaacaacaacatgcggcccaagccccctcgaagctgtagaggaggtggaaggactagaggttagaggagaccccgcatttgcatcaaacagcatattgacacctgggaatagactgggagatcttctgctctatctcaacatcagctactaggcacagagcgccgaagtatgtagctggtggtgaggaagaacac

SEQ ID NO: 25 - DNA, 11014 bp, Murray Valley encephalitis virus
agacgttcatctgcgtgagcttccgatctcagtattgtttggaaggatcattgattaacgcggtttgaacagttttttggagcttttgatttcaaatgtctaaaaaaccaggaggacccgggaagccccgggtcgtcaatatgctaaaacgcggcataccccgcgtattcccactagtgggagtgaagagggtagtaatgaacttgctagatggcagagggccaatacggtttgtgttggctctcttagctttcttcaggtttacagcacttgccccgaccaaggccttgatgaggcgctggaagagcgtgaacaagacaacggccatgaaacatctgaccagttttaagaaagaattaggaacactgattgatgtggtgaacaaaaggggcaaaaaacaaaagaaaagaggtggcagtgaaacatccgtgcttatgctcattttcatgctgattggatttgccgctgccttaaagctttccaccttccagggcaagataatgatgactgtgaacgctacggacattgctgatgtgatcgccattccaaccccgaagggacccaatcaatgctggattcgagccattgacattggatttatgtgtgatgacaccatcacttatgaatgcccgaaattggaaagtggaaatgaccctgaagacattgactgctggtgtgacaaacaagctgtgtacgtaaactatggaaggtgcacacgtgctcgccattcaaagcgcagtcgtcgttccatcacagtgcagactcatggtgaaagcactttggtcaacaaaaaggatgcctggctggattccacgaaggccacgcgttatctcaccaaaacagagaactggattataagaaatcctggttacgcgctggtggccgttgtccttggctggatgctgggcagcaacactggacaaaaagttatttttacagtgcttttgctcctcgttgctcctgcctacagttttaactgtctgggaatgagcagccgtgatttcattgaaggtgcttcaggagctacatgggtcgatttggtgctggagggcgacagttgcatcaccatcatggccgctgacaaacccacccttgacataagaatgatgaacattgaagccaccaatcttgcactggttagaaattactgctatgcagctactgtgtcagacgtttctacggtgtcaaactgtcccactacaggggagtcacacaacacgaagcgggcagatcacaattacttgtgcaaacgaggtgtgaccgacagaggctggggtaatggatgtggcttgtttggtaaggggagcattgacacatgcgcaaagttcacctgctctaactcagctgcggggagacttatcttacctgaggacatcaaatatgaagttggggtttttgttcacggatcaacggactcaaccagtcatggaaattattctacccaaattggagctaaccaagcagtcaggttcaccatttcaccaaacgctccagccatcacagcaaagatgggcgactatggagaagtcactgtggagtgtgaaccgaggagtggactgaatacagaggcctactacgtcatgaccattggaacgaaacactttctagtgcatcgtgagtggttcaatgatttgctcttgccatggacatcacctgcaagcacggaatggaggaatagagaaattctcgtggagtttgaagagccacatgccaccaaacaatcagtggttgccttgggttcacaggaaggagctttgcaccaagctctggctggagccataccagtcgagttttcgagcagcacacttaaactcacttcaggacaccttaagtgtcgcgtgaaaatggagaaattgaaactgaaaggaaccacttatgggatgtgcacagaaaaatttactttctcaaagaatccagccgacaccggtcatggcacggtagtactagaactgcagtacaccgggagtgatggaccatgcaaaattccaatatcctctgtagcaagtctcaatgacatgacgcctgtcggaagaatggtgacagctaatccatatgtagcttcatcaactgccaatgctaaagttctggtggagattgaaccacccttcggagactcatacattgtggtaggcaggggagacaagcagatcaatcaccactggcataaggagggtagttcaattggcaaagccttcagcacaaccttgaagggagcacagagattggcagctcttggagacacggcgtgggactttggatcagtaggcggagtcttcaattcaatcggaaaggcagtacaccaagtctttggaggagcatttagaaccctctttggaggaatgtcatggatcagcccaggtctgctgggggcgttactgctatggatgggggtcaatgctagagataaatcaattgctttggctttcctagcaacaggaggcgttttgttgttcctggccacaaatgtccatgctgacactggttgcgcgattgacatcaccaggagggagctcaagtgtggcagcggaatattcatacacaatgatgttgaagcctggattgaccgctacaagtacctgccagagacccccaagcaactggctaaagtggttgaaaacgctcacaagagcggaatatgtgggatacggtcagtgaatagatttgaacatcaaatgtgggaatctgtgcgtgatgaactcaatgctttactcaaggaaaatgccattgatttgagtgttgtcgtggaaaaacagaaaggcatgtacagagcagcacccaataggctgagactcactgtggaagaacttgatataggctggaaggcctggggtaagagtttgctttttgcggcggaattggccaattcaacgtttgtggttgatggacctgaaacagctgaatgtcctaattcaaagagggcatggaacagctttgaaattgaagactttggatttggcataacatccaccaggggttggcttaaactcagagaggaaaatacctcggagtgtgacagcaccattattgggaccgcagttaagggcaaccatgcagtacacagtgacctttcctactggattgagagtggactcaatgggacatggaaacttgagagagccatttttggagaggtaaaatcctgcacctggcccgagacacacacactatggggtgatgcagtggaagaaacggagttgataatcccagtgactctcgccggcccacgcagcaagcacaacagaagagaaggatacaaggttcaagttcaaggtccgtgggatgaagaagacataaaattggactttgactactgcccaggaacaaccgtcacagtgagtgaacattgtggaaaacgaggtccctcagtacgcaccacaactgacagcgggaaactcgtcacggactggtgttgtaggagctgcacgcttcctcctttaagatttaccacggccagtggatgttggtatggaatggagataagacctatgaagcatgatgagtccactctagttaaatcaagggttcaagcatttaatggagatatgattgatccttttcagttaggccttctggtgatgtttctggccacccaggaggtcttgaggaagaggtggacggccagacttactctgccagcagcggttggggctctgctagtcctcctccttgggggcattacctacactgatctagtgcggtatctcatattggtgggttcagcatttgcagaatcaaacaacggaggtgatgtcattcatttggcactcattgctgtattcaaggtacagcccgcttttcttgttgccagcttgacacgcagtagatggactaatcaagagaatctcgtcctggtcttgggagcggccttctttcagatggcagcttcagacctggagttgacaattccaggtttgttgaactcagctgctacagcttggatggtgttgcgagcaatggcttttccgtcaacctcagcaatagccatgcccatgctagcaatgcttgctccgggaatgagaatgcttcaccttgacacatataggatagtgctcttgctgattggcatttgcagcttgctgaatgagagaaggaggtctgtggagaaaaagaaaggagctgttttaattggtctagccctgacttcgactggatatttttcaccaacaatcatggcagcaggccttatgatatgcaaccctaacaagaagagaggatggcccgccacagaggtgttaacagcagtgggactgatgttcgccattgttggaggactcgctgagctagacattgattctatgtcagtcccttttaccatagctggcctcatgttagtgtcctatgttatctcgggaaaagccacagacatgtggcttgaaagagcagcagatgtgtcatgggaagcgggggcagcaataacaggtaccagcgaaagactggatgtccaattggacgatgatggagactttcatctgctcaatgaccctggagttccatggaaaatctgggttttaagaatgacatgcttaagcgtagctgccatcactccaagggccattttgccatccgcgtttgggtattggctgactctgaagtacacaaaacgagggggtgttttttgggatacgccttccccaaaagtgtatcccaagggggatacgacgccaggagtctaccgaataatggccagaggtatcctgggaaggtaccaagctggggtaggagtcatgcatgagggtgtgttccacacgctgtggcacacaactagaggagctgccataatgagtggtgaaggaagattgacaccatactggggcaatgtgaaagaggacagagtgacctatggtggcccatggaaattagatcaaaaatggaatggagtggatgacgtccagatgatagtggtggaaccaggaaagccagcaataaacgtgcagaccaagcccgggatttttaagacagcacatggagaaattggagccgtgagcttggattacccgattggaacttcaggatccccaatagtcaatagcaatggagaaatcattggcctttatggaaatggagtgatactaggtaatggggcctacgtcagtgctattgttcaaggagaaagagtagaggaaccagtgccagaggcctacaatcctgagatgctaaagaaaaggcaactaaccgtgctggacctgcacccaggtgctgggaaaacacgacgcatactcccccaaataattaaagatgccatccaaaaaagactacgaacagctgttcttgcaccaacgagggtggttgcagcagaaatggcagaagctttgaggggccttccagttaggtatttgactccagctgtacaaagagaacacagtggaaacgagatagtggatgtgatgtgccatgcgacactaacacatcgattgatgtcgccgctaagagtcccgaattacaacctgtttgtcatggatgaagcccatttcacagatccagccagtatagcagctcgtggatatattgccacccgtgttgaagctggagaggccgcggccatctttatgacggccacccctccaggcacatctgacccattcccggacaccaattcaccagtccatgatgtatccagtgaaatcccagacagagcatggagcagtggatttgagtggataaccgactatgctggaaagaccgtctggttcgtggcaagcgtaaaaatgagcaatgagatcgcgcagtgcttgcaacgagcagggaagcgagttatccaattgaacaggaagtcctatgatacggaatacccgaaatgcaaaaatggtgattgggactttgtgataacaactgacatctcggaaatgggagcaaattttggggcaagcagagtgattgattgtcgcaagagtgtcaagccaacgatcttggacgaaggagagggaagagtcattctcagtgtgccgtcagccatcactagtgccagtgctgcccaacggagaggtagagtgggcaggaatccaagtcaaataggagatgagtaccactatggaggcggaaccagtgaggatgacaccatgctggcgcactggactgaagcaaaaattcttttggacaacatccacctcccaaatggcctagtagctcagctgtacggtcccgaaagagataaaacttacaccatggatggagagtaccgactcagaggcgaagaacgcaagacattcctcgagctcataaaaacagccgatttgccggtgtggcttgcttacaaggtagcgtcaaatggcatccagtacaatgacaggaaatggtgttttgatggaccccgatccaacattatcctcgaagacaacaacgaagtggaaatcataacaagaataggagaaagaaaagttttgaagccacgatggttagatgcacgcgtgtattcagaccaccagtccttaaaatggttcaaggactttgcagctgggaagcggtcggcgatcggattttttgaagtgctaggccggatgccagagcattttgcagggaaaacacgtgaagctctggacaccatgtacctggtggcaacatcagaaaaaggaggcaaagcgcatagaatggctcttgaagaactacctgatgctctggaaaccataacactcattgcagcgttgggagtcatgaccgcagggttttttctcctcatgatgcaaagaaaaggaattggtaaactgggactcggagcactcgtcttggtagtggccacttttttcctttggatgtcagacgtttctggcaccaaaatagctggtgtgttgctgcttgcgctgcttatgatggttgtgttaatccctgaacctgagaaacagcgttcccagacggacaaccaactggcagtattcttgatatgtgtgctgcttgttgttgggttggtggctgcaaatgaatatgggatgttggaaaggacaaaaactgacataagaaacctatttgggaagtcactaattgaggaaaacgaggtccatatccctccctttgattttttcaccctggatttgaagcctgctactgcttgggctttgtacggaggaagcacagttgtgttgactccactgataaaacatctggtgacatcacagtatgttaccacctcattggcatcaataaatgcccaagcagggagcttgtttacattgcctaaaggaattcctttcactgattttgacttgtctgtagcactggtgtttcttggttgctggggacaagtgacgctcaccaccttgataatggcaacaatactggtaaccctccactacggatatcttttacctggttggcaagcagaggctttgagggctgcacagaagcgcacagccgctggtatcatgaaaaacgcagttgttgatggcatagtcgcgacagatgttccagagctggaaagaacgaccccacagatgcagaaaaggctaggccagattctactcgtcttggccagcgtggcagcagtatgcgtgaaccccagaataactaccatccgtgaagctggaatactctgtaccgcagcagctctcaccctgtgggacaacaacgccagcgcggcttggaattccaccacggctactgggctgtgccatgttatgcgtggaagctggatagcgggagcttcaatagcctggactctcataaaaaatgcagaaaaaccagcgttcaaaagaggaagagcaggaggaagaactctcggagagcaatggaaagaaaagctaaatgccatgggtaaagaagaattcttcagctatcggaaagaagccatcttggaggtggatagaaccgaagccagaagggccagaagagagggaaacaaggtaggaggccacccagtgtcacgagggacagcgaaactcagatggttggttgaaagacgctttgtccagcccataggaaaagtagtggatctggggtgtggcagagggggctggtcatactacgcagcaaccatgaagaacgtccaggaagtgagaggatacacgaaaggtgggccaggccatgaagaacccatgttgatgcagagctatgggtggaacatcgttactatgaaaagtggcgttgatgttttctacaaaccatctgaaatcagtgacacccttttgtgtgacataggagagtcatcacctagtgctgaaatagaagaacaacggaccctgcggattttggaaatggtgtctgactggctaagccgtggtccaaaggaattttgcataaagatactctgcccgtacatgcccaaggtgattgaaaaactggagagcctgcaacgcaggtttggaggtggattggtacgcgtccccctttctcgtaactccaaccatgagatgtactgggtcagtggagcctcggggaacatcgtgcacgcggtgaacatgaccagtcaggtgctcattggaagaatggacaagaagatttggaaaggcccaaaatatgaggaagatgtcaacctgggcagcggcacgagagcagtaggaaaaggtgtgcagcacacggattacaagaggatcaaatccagaatagaaaaactgaaggaggagtatgcagccacgtggcacacggatgataaccacccgtatagaacttggacttatcatgggagttatgaggtgaagccatcggggtcagccagcacgttggtaaatggagtggtgcggcttttgagcaaaccatgggacgctatcactggagtcaccaccatggccatgactgataccacaccatttggccagcagcgagtgttcaaagagaaggttgacacaaaagctcctgagcctccccaaggagtgaagactgtgatggatgagaccaccaactggctgtgggcttatttggctaggaacaaaaaagccagactgtgcaccagagaggagtttgtgaaaaaagtgaacagccacgccgccttgggagccatgtttgaagagcaaaaccagtggaagaacgcacgagaagctgtggaagacccaaagttctgggaaatggtggatgaggaaagggaatgccacctccgaggggaatgccgaacctgcatatataacatgatgggcaaacgtgagaaaaaacccggagaatttggaaaggccaagggaagcagagccatctggttcatgtggctgggagccagattcttggagtttgaagctctaggattcctcaatgaagaccattggatgagtagagagaattcaggaggaggagttgaaggagctggtattcagaagctgggatacatcttgagagatgtggctcaaaagcctggagggaaaatttatgccgatgacacagctggttgggacacccgcatcacacaagctgaccttgagaacgaagctaaagttctggagttgatggaaggtgagcagcggactttggcaagagcaatcattgagctgacatacaggcacaaagtggtcaaggtcatgcgcccggcagctggaggaaagactgtgatggatgtcatttcacgcgaagatcaaagaggaagtggacaagtagtgacctatgctttaaacacattcaccaacatcgctgtgcaattggttagactcatggaagccgaagcggtcataggtccagatgacattgaaagcattgaaaggaaaaagaaatttgcagtccgcacatggctttttgagaacgcagaagaaagagtgcagcgcatggctgttagtggtgatgactgtgttgtcaaaccattggatgatagattttccactgctttgcatttcttgaacgccatgtcaaaggtgagaaaagacatccaggagtggaaaccctcacaaggctggtatgactggcaacaagtccccttctgttcgaatcatttccaggaagtcatcatgaaagatgggaggaccttggtggtgccctgcagaggacaggacgagctgattggaagagcacgaatatcccctggctcaggatggaacgtcagggacacggcatgcttagcaaaagcatatgcacagatgtggcttgtgttgtacttccatcgacgggatctgcgcctgatggccaacgccatatgctcttcagtcccagttgactgggttccaaccggcaggactacttggtccatccatggaaaaggagaatggatgacaacagaagacatgctaagcgtgtggaaccgtgtgtggattttggaaaatgaatggatggaggacaagaccacggtttccgactggacggaagtcccatatgtagggaagagagaagacatctggtgcggaagtctcatagggaccaggaccagggcaacatgggctgagaacatctacgccgccatcaaccaagtgaggtcagtcatagggaaagaaaaatatgtagactacgtgcagtcgctgaggaggtatgaagaaactcatgtgagtgaagatagggtcttgtaaataacattgatagaaaattttgtaaatatttaatgtaatatagtataggtaaaattttttgaaattaagtaaaattaagtagcaagacttgatagtcaggccagccggttaggctgccaccgaaggttggtagacggtgctgcctgcgaccaaccccaggaggactgggttaccaaagctgattctccacggttggaaagcctcccagaaccgtctcggaagaggagtccctgccaacaatggagatgaagcccgtgtcagatcgcgaaagcgccacttcgccgaggagtgcaatctgtgaggccccaggaggactgggtaaacaaagccgtaaggcccccgcagcccgggccgggaggaggtgatgcaaaccccggcgaaggactagaggttagaggagaccctgcggaagaaatgagtggcccaagctcgccgaagctgtaaggcgggtggacggactagaggttagaggagaccccactctcaaaagcatcaaacaacagcatattgacacctgggaaaagactaggagatcttctgctctattccaacatcagtcacaaggcaccgagcgccgaacactgtgactgatgggggagaagaccacaggatct

SEQ ID NO: 26 - DNA, 10647 bp, Powassan virus
tttcttgcacgtgtgtgtgggcactttagtcagtgtccgcggcgttcttttgaacgtgagtgtgttgagaaaaagacagcttaggagaacaagagctgggagtggtcatggtgactacttctaaaggaaaggggggcggtccccctaagcgaaagctgaaagtgaccgcaaacaagtcgcgaccagcaacaagcccaatgccaaagggttttgtgctgtcgcgcatgctggggatcctttggcatgccatgacaggcacggccaggaccccagtgttgaaaatgttctggaaaacggtaccattacgccaagcggaatctgcgttgaagaagatcaagagagtcattggaaacttgatgcagagcctccacatgaggggacgtcgcagatcaggcgtcgactggacatggactttcttggtaatggctttgatggctatggccatggcgacctccatccatcgggataaagagggatatatggtcatgcgagctagcggaagagatgccgcaagccaagtcagagtccaaaacggaacatgcgtcattttggcaacggacatgggagaatggtgtgaagattcaataacctactcctgtgttaccattgaccaggaagaagaacctgttgatgtggattgtttttgccgaggtgttgacagagtcaagttagagtacggacgctgtggaagacaggctggattcagggggagaaggtctgtggtcatcccgacacatgcacaaaaggacatggttgggcgaggacacgcgtggctcaagggtgataacatccgagatcatgttacccgcgtcgagggctggatgtggaaaaacaagctcttgacagttgctgtcgtggctttggcttggctcatgctggatagttggatggctagggtgactgtgatccttctggcgctgagccttgggcctgtatacgccacgagatgcacacaccttgagaacagggactttgtaacaggagtacagggaaccactagggtatccctggttctggagcttggaggatgtgtgaccattactgctgaaggcaagccatctattgatgtatggctggaggacatttttcaagaaagtcctgctgaaaccagagaatactgtttacacgccaagctctccaacacgaaagtagaggctcgttgccccaccaccgggccagccacgcttccagaggaacaccaggccaacatggtgtgcaagagagaccagagtgaccgtggatggggaaaccactgcggattttttgggaaaggaagcatagtggcctgtgccaagtttgaatgcgaggaagcaaaaaaggctgtaggccatgtttatgactcaacaaaaatcacgtacgttgtcaaggttgagccccacacgggggactaccaagctgcaaatgaaaccaacgagaacaggaaaacggcccagttcacggtggcatctgagaaggtgattttaaatctaggagactatggagatgtctcattgacatgtaaggtggcaagtggaattgatgtcgcccagactgtggtgatgtcgcttggcagcaacaaagatcacctgccttcggcatggcaggtgcatcgtgactggttcgaagacctagccctgccttggaaacacaaagacaatcaggactggaacagtgtggagaaacttgtggagtttgggccgccccatgcagtgaaaatggatatcttcaatctgggggatcagactgctgttttgctcaaatcactggctggagtcccactggcaagcgtggacaaccagaaatatcacctgaaaagtggtcatgtcacctgtgatgtgggacttgaaaagctaaagctgaaaggcacaacttactccatgtgtgacaaaacaaagttcaagtggaaaagagttcccgtggacagtggtcatgacacagtggtcatggaggtgtcttacacgggaagtgacaagccctgccggattccggtacgagcagtggcacatggtgtcccgacaattaatgtggccatgctcattacccccaacccaaccatcgaaacaagtggaggaggatttatagagatgcagttgccaccgggggacaacatcatctatgtgggggacctaagtcagcagtggttccagaaaggcagtaccataggtagaatgctcgaaaagacccgtaagggtttggaaagactctcagtggttggagaacatgcatgggactttggttcagtgggtggaatcctgtcgtcagtggggaaagccgttcacacagtgttgggaggggcgttcaacaccctttttgggggagttgggttcatccctaagatgctgttgggagtcgccttggtgtggttggggctcaacgctaggaatccaacaatgtccatgacttttcttgccgtgggggttttgaccctgatgatgacaatgggagtcggagcagactatggatgtgcagttgatccagagaggatggaaatccgatgtggagaaggcctagttgtgtggaaagaggtatcggagtggtatgacggctatgcttaccatccggaatcaccagacacgctggcgcaggccctccgagaggcctttgagagaggcatctgtggagttgtgccccagaatagacttgaaatggccatgtggaggagcaccgctccggagctcaatttggtgttgagtgaaggagaggcgaatctgaccatcgtggtagacaagacagatccagcagattacagaggaggcactccaatggccctgaagaaaactggaaaggagagtaaggtgtcatggaagagttggggcaagtcaatcttgtggagtgtgcctgaaagtcctaggcgcatgatgatgggggttgatggagctggggagtgccccctgcacaagagagcaacaggagtgttcacagtggcagaatttggagttggcctacgcacaaaggtgtttttagacctgcgtggagaggcttcaagagagtgtgacacaggagtgatgggggcagctgtgaagaacggcaaggccattcacacggatcagagcatgtggatgagttccttccggaatgatactggcacgtacattcaggagctcattcttactgaccttcgaaactgtacatggccagcaagtcacaccattgacaacgacggagtcttggactcccgtctttttctgcctgtcactctcgctgggccccgttcaaaatacaacagaatacctggatacagcgagcaaacgaaagggccatgggaccagacccctctgagggtggtccgggatcattgccctggaacaagcgtgaagattgacagtcagtgtgacaagagaggagcctcagttagaagcaccacagaaagcggaaagatcatcccagagtggtgctgtcgggcttgtgaattaccaccggtgacgttccgcagcggcacagattgttggtatgccatggagattagaccggttcactcacagggtggcttggttcgatccatggtaatggctgacaacggtgcgatgctcagcgaggggggaatccctggactggtggcggtatttgtcctgatggagttcttgttgagaaggcgtccgggttctgctacatccattctgtggggaggcatactcatgctgggcttgcttgttacagggcttgttagagtggaggagatcgtgcgctacatcatagcggttgggatcactttccacttggagctgggaccagagatcatggcccttgtgatgctgcaggcagtttttagcatgcgcacatgctatctcattgggtttctcgtcaagagggtgatcaccacacgtgaggtggtgactgtttacttcctactcttggtcctagagatggggataccagagatgaactttggacatttatgggagtgggctgactcattggccatgggactactcataatcaaggcaagctcgatggaagaccgaaaaggtctaggattcttattggctgggctcatgacacaacgacatctgactgctgttcaccatggattaatagtattcctcactgtggctctggcggtggtgggacgaagcatctacaacgggcaaaaagagaggaaaggattaagctttaccatccccttggcctcgctattgggggggcctggatctggcttgcgcatgctggccttgtgggaatgcctgggtggtcgtggcagacgaagtctgagtgagcccctaactgtggtgggggtcatgttagcaatggctagtgggctgttgcgtcattcttcccaggaggctctactggtcctctcggctggttcctttctcattctgatgttgatcttggggactcgcagactccagttgacggcggaatgggcgggactggttgaatggaaccctgagttggttaacgaggggggtgaggttagcctaaaggtcaggcaggacgccatggggaacttgcacttgacggaggtggaacgggaggagcgcaggctggcactgtggctggcatttggcttgctagcttcggctttccattggtccggaatactggtgacaatgggggcatggacactgtatgagctcttctcatcagcacgcagaactgaccttgtattctcagggcaattgtctgaccatggggagaaacggccttttgacatcaaagatggcgtctatcgcatatacgcccctggactcgtatggggtcaccgtcagattggagtggggtatggaacaaaaggagtcctacacaccatgtggcatgtgacaagaggagcggctcttagtgtggagggggccgtctcgggaccatattgggcggatgtgcgtgaggacgtggtctgttatggaggtgcctggagcttggaaggaaaatggggaggtgaagttgttcaggtccatgcttttccaccagacagtggacataaagttcatcaatgccaaccagggaaactgaatcttgagggagggcgagtcatgggggccatcccaatagacttacctcgtggaacctcaggcagcccaatcatcaacgcacaaggggttgtgttgggtctctacggtaacggtctgaaatccaatgatgtttacatcagcagcatcgctcaaggtagcgttgagaaaagccgaccagaaatgccgttggcaatccaggggggcagatggacatcaaaaggcagcatcacagtcctggacatgcatcctggatcgggaaagacacatagagtccttcccgagctgattcgcgaatgcattgacaaaagattgaggactgttgtgttggccccaacgcgggttgttctgaaagagatggaaagagccctgcaagggaaacgtgtcaagttccactcggctgccgttgacaacacctcttcatcattgggggccattgttgatgtcatgtgtcacgccacttatgtcaatcgccgactcttgccacaggggagacagaattgggaggtggccataatggatgaagcacattggactgatcctcacagtatagcagcacgtggtcacctgtactctttggctaaagagaacagatgcgcgtttgttctgatgacagcaactccaccgggcaagagtgaggcattccccgagtccaaaggagccatagtgagtgaagaaagacctattcccgatggggagtggagagatggctttgattggataacggaatttgaaggccggacggcttggttcgtgtcatcaatagccaaaggaggcgctatagcacgaacactgaggcagaaagggaaaagtgtgatttgtttgaacagcaagacttttgacaaagactatggcagagtccgtgaagaaaaacctgactttgttgtgacgaccgacatctcagaaatgggggccaatttggacgtcaacagggtcattgatggcaggaccaatatcaagcctgaagaaatagacggaagagtggaactggccggcacaaggcgagtaacaacagcatcagcggcacaacgcagggggcgggtcggaaggcatgaaggacgcacagacacttatgtgtattctggacaatgtgatgatgatgacagctctctggtgcagtggaaagaagcccagatcttactcgacaatattacaacagtgcggggaccagttgccaccttctatgggccagaacaagggaaaatgcttgaggtggccggacactttcgtttgacggaggagaaaaggaaacatttcagacatctcttaacaaactgtgacttcacaccctggcttgcatggcacgtggctgcaaacaccacctgcgtgacagaccggaaatggacctgggagggtcccgaggagaatgccattgatggtcctgatggtgaactggttactttcagaagccccaatggggctgagcggaaattgaaaccagtctggaaagacagtagaatgttccgggaaggaagggatgtggcagactttctccaatacgcttctggccgcaggagtgccatggacatcttcactggacttggtggagtaccggacttgttgcgcttgcgctgcacggcagcatgggatgtcgtctacacattgctcaatgagaccccaggttctcgggccatgaagatggcagagagggacgctcccgaggccatgttgacgcttctggaggtggcggtgctaggcatcgccacactaggggtggtctggtgcttcattgtccgcacgtcggttagcaggatggttctgggcactgtggtgctggtggtggccctcattctactgtggctgggaggaatggactatgggaccatggcaggagtggcccttgttttctatttgctccttacggttttacaaccagaaccaggaaaacaacgcagtggggaagacaaccgactggcttttctgctgattggactggggagtgtcataggtctattggcggctaatgaattgggctatttggagagaacaaaagccgacattgcaggccttttcagatatgacacccagggtgacagagtgtgggacacatggacaaacattgacatacagcccgcccgatcctgggggacctatgtgtttatcgtgtcccttttcactccgtacatgctccatcagctgcagaccaaaatccagaggctggtgaacagctcggtagcagctggaacgcaggctatgaaagacttgggaggaggcaccccatttttcggtgtggctggacacgttgtggcacttggtgtgacttctttggtaggagcgacgcccacttctctggcattgggagtggctttggccatcctccatctggctgtggtcacatctgggctagaggctgaattgactcagagagcgcacagagcgttcttttcggccatggtgaagaatccaatggtggatggagaggtcatcaatcccattcccgatggagagccaaagccagccttgtatgaaagaaagatgagcttgctccttgccgttggcttgtgcatagctgctgtggccctgaacaggaccgcagcggcgatgactgaagccggtgcagtggccgtggccgctctgggacagttgttgaggccagaggaggaaagctggtggaccatgccaatggcttgcgggatggctgggctggtgcggggaagtctgtggggcctactgccggttctccaccgcatctggctcagaactcagggggctagaaggggtggagcagagggaagcacccttggcgacatctggaagcaacggttgaactcatgcaccaaagaggaattttttgcttacagacgcacaggagtcatggagaccaaccgagaccaggcccgcgagctgctccgcagaggggagaccaacatgggtttggccgtatcacggggttgcgccaaacttgcatggttagaagagagaggctacgcgacactcaagggagaagtcgtggaccttggctgtggaagggggggctggtcatattacgccgcctcgcgaccatctgttatggccgtgaaagcttacaccattggagggaaaggccatgaggcgccgcgattggtgacgagccttggatggaacctcataaagttcaggagtgggatggacgtcttttcaatgccagccacaagagtagatacaatcctgtgtgacatcggggagagcaatccggatccagagaaggaggggactcggagccggagggtgatccttttgatggagcaatggaaagccaggaatccagacgccgctgctgttttcaaggtgttggccccgtacagaccagaagtgctcgaggccctacaccgttttcagctacagtggggtggaggacttgtgagagtccctttctcacggaattccacacatgagatgtattattccacagccgtcacgggaaatcttgtcaattcggtcaatgtgctctcacgaaagcttcttgcgagatttggggagacaaggggacccatccaggtgcccgagattgatctaggaaccgggaccaggtgtgtgacactggcagaagacaaggtgaaacctagtgatgtgatggagcgcattggggccctgaaggaacaatactcagaatcatggcatgaagacaaggaacacccctacagaacatggcagtattggggtagttaccgcactcctgccactggatcagcggcttcacttatcaatggagtggtgaagttgttgagctggccttggaatgcccgagaggatgttactagaatggccatgacagacacaacagcgtttgggcaacagcgcgttttcaaagaaaaagtggacacaaaggcccaggagccacagccaggcaccagagtgatcatgagagcggtgagtgactggctacttgagcaccttcacagccgagccagggtgaggatgtgcaccagagatgaattcatagcaaaggtgagatccaacgcagctctgggcgcatggtcggatgaacagaataaatggagctcagcgaaagaagcagtggaagacccagagttttggaacctggtggatgaagagcgctctagacacctgaaaggccaatgtcgacattgtgtgtacaacatgatgggaaagagagagaagaagctgggtgagtttggagtcgctaaaggcagcagggccatctggtacatgtggcttgggagccgtttccttgagtttgaagtcctaggtttcctcaacgaagaacactgggcttccagggtggtcagtggagccggagtggaaggaaccagcctcaactatcttggttggttgctcagggagcttggagcaaaagatggcggcaggttatacgccgatgacactgctggatgggacacccgaatcaccaacgcagacttggaggatgaagagcagatccttcggtacatggaaggagaacaccatgtgctagcaaaaacaatactggaaaaagcctatcacgcaaaagtggttaaggttgccaggccctcccctcaaggaggatgtgtgatggatgtgatcacgagaagagaccaaaggggatcgggacaggttgtcacgtatgccctcaataccataacaaacatgaaagtccaactaatccgcatgatggaaggagagggagtcatcggaccagcagactcacaggatccccgtctgaaaagggtagaagcttggctgaaggagcatggagctgaacggttggggaggatgctggtgagtggagacgactgcgtcgtcaaacccatcgacgacaggtttggaaaggccctgtacttcctgaatgacatggcaaaaacccgcaaggatgtgggcgagtgggagccctcagtggggttcacagactgggaagaagtgcccttctgctcacaccatttccatgagctcgtgatgaaggatgggcgttccctcatagtgccctgcagagatcaagacgagctggtcggcagagcacgagtgtcgccaggatgtggatggagtgtgcgggagacagcctgcctgtccaaagcgtatgggcagatgtggctcctcaactacttccatcgcagagatctccgtacccttggacttgccatatgctctgcggttcccgtgaactgggttccgatgggccgcaccacctggagcatccatgcgagtggtgagtggatgacgacagaagacatgctacgcatctggaacaaggtctggattttggacaaccctcacatggaaaacaaacaaatggtggatgaatggagagatattccctacttgccaaagacacaggatctggtatgttcctcacttgttggaaggaaggaaagggctgaatgggccaagaacatctggggctcagtggaaaaggtgaggaaactcattggagcagaggaatacagagactatttggcaaacatggacagacatgatctgcactgggagctcaagctagagagctcaataatctaaactagcacaaccaaataatcagaagaaacccactacagggggtgatgtggcagcgcaccacgacatcgtgacgggaagaggtcgtccccgacgcatcatctctctagggcattttcgtgagaccctcatggctgctaaaggggcatcagccgtgtagtaagaaggccccgacctactgcagcagcacacacagtgacgggaagttggtcgctcccgacgcagttaggtcagagaaaattttgtgagaccaaaaggcctcctggaaggctcaccaggggttaggccattctag

SEQ ID NO: 27 - DNA, 10940 bp, St. Louis encephalitis virus
agatgttcgcgtcggtgagcggagaggaaacagatttcctttttggaggataataacttaacttgactgcgaacagttttttagcagggaattacccaatgtctaaaaaaccaggaaaacccggtagaaaccgggttgtcaatatgctaaaacgcggcgtatcccgcgtgaaccccttgacggggttgaagaggatacttggaagtctgctggatggccgtggacccgtgcggttcatactagccatcctgacattcttccgatttacagctctacagccaactgaggcgctgaagcgcagatggagggctgtggacaaaagaacggcattgaaacacctgaacggatttaaacgtgacctcggatccatgctagacaccatcaaccggcggccaagcaagaagagaggagggaccagatcgttgctcggattggctgcgctaattggactggcgagttcattgcagttatcaacctatcaggggaaagtgttaatgtcaatcaacaagactgacgctcaaagcgccataaacattcctagtgccaacggagcaaacacttgcattgtgagggctctagatgtgggggtcatgtgcaaagatgacatcacatacctgtgcccagtgctttcagcgggaaatgatcccgaggacattgactgttggtgtgacgtcgaagaggtgtgggtgcactacggcagatgtacgcgcatgggacattcgaggcgtagccgacggtcaatttctgtgcagcaccatggagattccacactggcaacaaagaacacgccatggttggacaccgtgaaaaccaccaaatacttgacaaaagtcgaaaactgggttttgcgcaatcctggatatgccctagttgcgctggcgattggatggatgctaggtagcaacaacacacagagagtggtttttgtgatcatgctgatgctgattgctccggcatacagcttcaactgtctgggaacatcaaacagggactttgtcgagggagccagtggggcaacatggattgacttggtacttgaagggggaagctgtgttacagtgatggcaccagagaaaccaacactggacttcaaggtgatgaagatggaggctaccgagttagccactgtgcgtgagtattgttacgaagcaaccttggacacgctgtcaacagtggcaaggtgccctacaacaggagaggctcacaacaccaaaaggagtgacccaacatttgtctgcaaaagagatgttgtggaccgcggatggggtaacggatgtggcctgtttggaaaagggagcattgacacatgcgctaagttcacatgcaaaaacaaggcaacagggaagacgatcttgagagaaaacatcaagtatgaggttgcaatttttgtgcatggttcaacggactctacgtcacatggcaattactctgagcagattgggaaaaaccaagcggctagattcaccataagcccgcaagcaccgtcctttacggccaacatgggcgagtatggaacagttaccattgattgtgaagcaagatcaggaatcaacacggaggattattatgtcttcactgtcaaggagaagtcatggctagtgaacagggactggtttcacgacttgaaccttccatggacgagccctgccacaactgattggcgcaacagagaaacactggtggaatttgaggaaccgcatgccaccaagcaaactgtagtagccctaggatcgcaagaaggtgccctgcacacagcattggctggagctattccagccactgttagcagctcaaccctaaccttgcaatcagggcatctgaaatgcagagctaagcttgacaaggtcaaaatcaagggaacgacatatggcatgtgtgattctgccttcaccttcagcaagaacccaactgacacagggcacgggacagtgattgtggaactgcagtatactggaagcaacggaccctgccgagttcccatctccgtgactgcaaacctcatggatttgacaccggttggaagactggtcacggtcaatccttttataagcacagggggagcgaacaacaaggtcatgatcgaagttgaaccaccctttggcgattcttacatcgtcgtcggaagaggcaccacccagattaactaccactggcacaaagaaggaagcagcattggtaaggctttggcgaccacatggaaaggagcccaacggctagccgtcttaggggacacagcgtgggactttggatctattggaggagttttcaattcaattggcaaagctgtccaccaagttttcggaggagcgttcaggaccctgttcgggggaatgtcctggatcacacagggactacttggagctcttctcctgtggatggggttgcaggcccgcgacaggagcatctcgctgactctactggctgttggagggattcttatctttctggcaaccagcgtgcaagccgattcgggatgtgcaattgacctacaacgacgtgaattgaaatgtggaggaggcatcttcgtgtacaacgacgttgagaagtggaagagcgactacaagtatttccctctgactccaactggactcgcgcgtgtgattcaagaggcgcatgccaatggcatctgtggcatccgatcaacgtcacggttggagcacttgatgtgggagaacatacagagagagctaaacgcaatctttgaagacaatgagattgacttgtcagttgtagtgcaagaagatcccaaatactacaaaagggctccccgacgcctgaagaagctggaggatgaattggactacggatggaagaaatggggaaaaaccctgtttgtggaacccagacttggaaacaacacatttgttgttgatggacccgagaccaaggagtgtccaacagcaaacagagcatggaacagtttcaaagtggaagactttggatttggcatggtgttcactcgattgtggctcacaatccgggaggaaaacacaacggaatgcgattcggcaatcatcggcacagctataaaaggagacagagctgttcacagtgacctatcgtattggattgagagcaaaaagaatgagacatggcaactggaacgagccgtcatgggagaagtgaaatcttgtacctggccagaaacgcacaccctgtggggtgatggtgttgttgaaagcgaaatgatcatcccggtgacattaggaggtccaaaaagccatcacaacaagagaaacggatactatacacagacaaaaggaccctggagtgaaggagaaataatcctggactttgactactgtccaggaacaacagtcaccgttactgaacattgtggaaacaggggagcgtcgctgcgcacaaccacagcgagtggcaagctggtcactgattggtgttgcaggagctgttccttgccaccactgcgatacaccacaaaagatggatgctggtatggaatggaaatccggccggtgaaggaggaggaagcaaagcttgtgaaatctcgagtgacagctggagttgcaggagggatggaaccattccaacttggtctcctggtagcattcatagccacacaagaagtgctgaaacgcagatggactgggaagctgaccctgacctcactagcagtatgtttggcactgttgatttttggaaacctaacatacatggacctggtgcgatacctggtccttgttggaactgcttttgcggaaatgaacactggaggcgatgtgatccacctagcacttgttgctgtcttcaaagtgcagccagcatttttggcgggtctgtttttaagaatgcagtggagcaaccaggaaaacatcttgatggttattggagcagcttttctccaaatggcggccaatgacttgaagcttgaggtccttccgatactcaacgccatgtccattgcgtggatgctgataagggcaatgaaagaaggtaaagtggcgatgtacgctcttcccatcttgtgtgctttgactccaggaatgaggatggctgggcttgatgtcatcagatgtttgttgttgataattgggattgtaacacttctgaatgaaagaagagaaagtgtggccaagaaaaaaggcggctacttgctcgcagcggccctctgccaagctggagtgtgcagtcccttgattatgatgggaggcttaatcctggctcaccccaacgggaaaaggagttggccggcgagtgaggtcttaactggagttggactgatgtgcgctttggcaggaggtcttctcgagtttgaggaaacctcaatggtggtcccttttgcaatagccggactgatgtatatcacatacaccgtctcaggaaaagccgcagaaatgtggattgaaaaagcagcagacatcacatgggaacagaatgccgagatcaccggaaccagccccaggcttgatgttgaccttgactcacatggcaacttcaagctcctgaacgaccctggagctccagtgcacttgtttgcgctgagattcatactcctgggcttgagtgcacgattccattggttcattccattcggagtactggggttttggctacttggcaaacactcgaagcgaggaggagcattgtgggacgtgccgtcaccaaaagtgtatccaaagtgtgagacaaaaccaggaatctatcgaatcatgactagaggaattttgggcactttccaagccggagtcggtgtgatgcacgaaggagtttttcacacaatgtggcacgcaactgaaggagcagtcctacgaaatggagaaggaagactagatccgtatgcaggagatgtccggaatgatctgatctcgtatggaggtccctggaagctgagtgccacttgggatggaactgaggaggtccaaatgattgcagtagctccggggaaaccagccattaatgtccagacaacaccaggagtgtttaagaccccgctcgggaccattggagccgtgacgcttgatttcccaaaaggaacatcaggttctccaattatcaacaagaaaggggaaatcattgggctctatggaaatggggtattgattggacagggcgaatatgttagtggcatcatccaaggggaaaggacagaagaaccaattccagatgcatacaatgaggaaatgctccggaagagaaaactaacagtactggaattgcatccgggagctggaaagaccagaaaggtgttgccccaaatcatcaaagattgcatccaaaaacgcttgcgaacggccgtgctagctcccactcgggtagtggcttgtgagatcgctgaagcgttaaaaggactgccaatccgttacctaacaccagccgtgaggaatgaacatcagggcaatgagatagttgacgtgatgtgccatgccacgctcacacagaaactgctgacaccgacccgggttcccaactaccaagtgtacatcatggatgaggcccacttcattgatccagcaagcattgctgctcgtgggtatatctcaacaaaagtggaattgggggaagcagctgccatctttatgacggcaactccaccaggaacaaatgacccttttccagattcaaattctcccatactggatgttgaggcccaggttccggataaggcgtggagcaccgggtatgagtggatcaccaacttcacagggaggactgtgtggtttgttcccagtgtgaaaagtgggaacgaaattgccatctgcctacaaaaagcaggaaagcgagtgattcaactaaacaggaaaagctttgacacagaataccctaagacaaagaacaacgaatgggattttgttgtgactactgatatatcagagatgggggctaactttggagcgcacagagtcatcgacagccgcaagtgtgtgaaaccagtcatcttggaagatgatgatcgagtcatcctcaatggacccatggcaatcacatcagcgagcgcagcacagcgcagaggccgcattggaagaaatccttcgcagattggagatgagtaccattatgggggtgcaaccaatgaggacgaccatgatctggccaactggactgaagcaaagattctccttgacaacatatacctcccaaacggactggtggcacaaatgtaccagcccgagcgagataaggttttcaccatggatggggagtttcgattgcgtggagaagaaaggaagaactttgttgagctgatgcgcaacggtgacctcccagtgtggttagcttacaaagtagcctctaatggacacagttaccaagaccgctcatggtgttttacaggacaaaccaacaacacaatcctggaagacaacaacgaagttgaagttttcaccaaaactggagacaggaaaattctcaggccaaagtggatggatgcgcgggtctgctgcgactaccaagcgctcaagtcatttaaagagtttgcagcagggaagagatcagcgctgggcatgatggaggtcatgggtagaatgccaaatcacttctgggaaaagacagttgccgctgcagacacactgtatctcctaggaaccagtgaggcaaacagtagggcacacaaagaggcactggcagaactgccagattccctggaaacgcttctcctgattggaatgctatgcgtcatgtcaatgggtacgttcatttttctcatgaacaggaaaggagttggtaaaatgggactgggagcatttgtgatgactcttgcaacggcattgctttgggcagcagaagttcccggaacgcagatagctggagttttgttgatagtgtttctattgatgattgtgttgattccagaacctgagaagcaacggtcccagactgacaatcaactggctgtcttcttgatatgcatcatgaccctcatgggtgtggtggcggcgaatgaaatgggccttttggaaaaaaccaagtctgacattgccaagttgtttggcagccaacctggatcggtgggatttgcaacaagaacaacaccttgggatatcagccttgacatcaaaccagccaccgcatgggcactgtatgcagcagcaacaatggtcatgacacccttgataaagcatctcatcacgacacagtacgtgaatttttcactaacagccattgcatcccaagctggtgtactattaggtttgaccaatggaatgccattcactgctatggatctatcggtgccattattggtgctagggtgttggaatcagatgaccctgccaagtttggccgttgcagtgatgcttttggctatccactatgcattcatgatcccaggctggcaggctgaagcaatgagagcagcccagcggagaacagcggctggcattatgaaaaacgctgtggtcgacgggattgtagcaaccgacataccagacctgtcaccggcgaccccaatgacagagaagaaaatgggtcaaatcctgctcatcgctgcagcagtgttagctgtgctcgtccggcctgggatctgttcgatcaaagaatttggggtcttagggtcagcagcccttgtgacgctaatcgaaggaacggccggagttgtctggaattgcacaacagcggttggcctctgtaacctcatgaggggagggtggttagctggaatgtctatcacgtggacagtctacaagaacgtggacaagcccaagggaaagcgtggaggaggaaaaggagccactcttggagagatctggaagtcaagactgaatcagttgacacgagccgaattcatggcatacaggaaagatggaatcgtggaagtagacagagcccccgccaggaaagccaggagagaagggcgtctcacaggtggccacccagtgtccagagggtcagcaaaattaagatggataacagaaagaggattcgtcaaacccatgggaaaggttgttgacttaggctgtggacgaggagggtggagctactactgcgcaaccctgaagcatgttcaagaagtgaaaggattcacaaagggcggaccaggccatgaagaaccgcagctcatgcaaagctatggatggaatctggtccacatgaaaagtggagtggacgtgttccataagccagctgagccagctgacactgtgctctgtgacattggagagtcaaacccaagctgtgaggtggaggaagcccgaacagcaagagtgttggacatggttgaggaatggttgaagaaaggagccactgagttctgcataaaagtcctgtgtccatacacgcccaaaatcattgagaagttggaaaaactgcaacggaagtatggaggaggcctggtcagagtccccttgtctagaaactcgacacatgagatgtactgggttagtggggcggctggaaacatcatacacgccgtcagcatgactagccaggtactcatggggaggatggacaaacagaacagaagtgggcctcgctacgaggaagatgtgaatttgggaagcggaacgcgctctgttggaaagctcacagagaagccagacccccggaaagttggggaaaggatacggagattgagggaagaataccaacaaacgtggacgtatgaccacaacaacccatatagaacgtggaactatcatggaagttatgaagtgaaaccaacyggatcagctagctcgatggtgaacggagtcgtgaggctcctttcaaaaccatgggatatgatcacaaacgtgaccactatggccatgactgacaccacgccctttggtcaacaaagggtgttcaaagaaaaggttgacacaaaggctcccgaaccaccactaggagtcgcccaaatcatggatgtgacaacagactggctgtgggacttcgttgcccgggaaaagaaaccaagagtttgtaccccagaagaattcaaagcaaaggtgaacagccatgcagctctgggagccatgtttgaagagcaaaatcagtggagtagcgcccgtgaagctgtggaagatccaaagttctgggaaatggttgatgaagaaagagaagctcatcttaaaggagagtgtcacacatgcatctacaacatgatggggaagcgtgagaaaaagactggagagtttgggaaggctaaaggaagcagagccatttggtacatgtggttgggagctcggttcttggagtttgaagcccttgggttcctaaatgaagaccactggatgagccgcgagaactcctatggaggagttgaggggaaaggactccagaaacttggatacatcctgcaagaaatctcccaaattccaggaggaaagatgtacgcagatgacacagccggctgggacacccggatcacaaaagaagacttgaaaaatgaagcaaaaataacaaagagaatggaagaaaggcatagaaagcttgcggaggcgatcattgatctaacctaccgccataaggtggtgaaagtcatgagaccagggcctgatggtaaaacctacatggatgtcatctccagagaagaccaaaggggcagtggacaggtggtcacatatgccctgaacaccttcaccaacctggccgttcaactgataagatgcatggaggctgaaggggtggtagatgaggatgacattacgagagtgagacttggacggttggccaaagcggtcgagtggctgaggaagaatggacctgaacgattgagcagaatggcagtgagtggggacgactgtgttgtgaaaccaattgatgacagatttgccactgcactccactttctcaacaacatgtcaaagatcagaaaggacattcaggagtggaaaccttcaacaggctggcacaactggcaagaggtgcccttctgctcacaccacttcaatgaactaatgctgaaggatgggcggaccatcgtggtgccatgtaggagccaggatgagctgattggcagagccagaatctcaccaggagcaggatggaatgtgaaagagacagcctgcttgagcaaatcctatgctcagatgtggttgcttatgtacttccacaggcgggacctcagaatgatggcaaatgccatttgttcagctgtcccagtcaactgggtcccaactggacgtaccacatggtcaattcatgggaagggcgaatggatgacaacagaagacatgttgagtgtctggaaccgtgtgtggattgaagagaatgaatacatgaaagacaaaacaccattggcggcttggaacgacattccatacctaggaaagagagaggacatctggtgtggaagccttatcggaacaaggaccagagccacctgggctgagaacatctacgctccaatcatgcagatccgcaacctcatcggggaagaggagtacagagactacatggttgcccaaaacagatttggtagggaagaaactcatgtggtaggaggagtgctgtagattgaaatgtaaatagtgtaaataaataaaaacaggtaagtcaggccaatcagttttgccaccggatgtcaggtaaacggtgctgtctgtaacctggccccaggcgactgggttatcaaagccaacccggctgggtgcaaagcccctcattccgactcgggagggtccctggcacgtaggctggagaggacgcacaagtcagaccagaaatgccacctgaaagcatgctaaaggtgctgtctgtacatgccccaggaggactgggttaacaaagcttaacagccccagcggcccaaaccatggagtgcgtgaccatggcgtaaggactagaggttagaggagaccccgctgtaacttggcaaggcccaaacccgctcaaagctgtagagacgggggaaggactagaggttagaggagaccccttgccgttaacgcaaacaacagcatattgacacctggaaagacaggagatcccctgctttttcaacaccagccacaaggcacagagcgccgtaaagtgtggctggtggtgaaaaaatcacaggatct

SEQ ID NO: 28 - DNA, 11141 bp, Tick-borne encephalitis virus
agattttcttgcacgtgcatgcgtttgcttcggacagcattagcagcggttggtttgaaagagatattcttttgtttctaccagtcgtgaacgtgttgagaaaaagacagcttaggagaacaagagctggggatggtcaagaaggccatcctgaaaggtaaggggggcggtccccctcgacgagtgtcgaaagagaccgcaacgaagacgcgtcaacccagagtccaaatgccaaatgggcttgtgttgatgcgcatgatggggatcttgtggcatgccgtagccggcaccgcgagaaaccccgtattgaaggcgttctggaactcagtccctctgaaacaggcaacagcagcactgcggaagatcaaaaggacggtgagtgccctaatggttggcttgcaaaaacgtgggaaaaggaggtcagcgacggactggatgagctggttgctggtcatcactctgttggggatgacgcttgctgcaacggtgaggaaagaaagggatggctcaactgtgatcagagctgaaggaaaggatgcagcaactcaggtgcgtgtggagaatggcacctgtgtgatcctggctactgacatggggtcatggtgtgatgattcactgtcctatgagtgtgtgaccatagatcaaggagaagagcctgttgacgtggattgtttttgccggaacgttgatggagtctatctggagtacggacgctgtgggaaacaggaaggctcacggacaaggcgctcagtgctgatcccatcccatgctcagggagagctgacgggaaggggacacaaatggctagaaggagactcgctgcgaacacaccttactagagttgagggatgggtctggaagaacaagctacttgccttggcaatggttaccgttgtgtggttgaccctggagagtgtggtgaccagggttgccgttcttgttgtgctcctgtgtttggcaccggtttacgcttcgcgttgcacacacttggaaaacagggactttgtgactggtactcaggggactacgagggtcaccttggtgctggaactgggtggatgtgttactataacagctgaggggaagccttcaatggatgtgtggcttgacgccatttaccaggagaaccctgctaagacacgtgagtactgtttacacgccaagttgtcggacactaaggttgcagccagatgcccaacaatgggaccagccactttggctgaagaacaccagggtggcacagtgtgtaagagagatcagagtgatcgaggctggggcaaccactgtggactgtttggaaagggtagcattgtggcctgtgtcaaggcggcttgtgaggcaaaaaagaaagccacaggacatgtgtacgacgccaacaaaatagtgtacacggtcaaagtcgaaccacacacgggagactatgttgccgcaaacgagacacatagtgggaggaagacggcatccttcacaatttcttcagagaaaaccattttgactatgggtgagtatggagatgtgtctttgttgtgcagggtcgctagtggcgttgacttggcccagaccgtcatccttgagcttgacaagacagtggaacaccttccaacggcttggcaggtccatagggactggttcaatgatctggctctgccatggaaacatgagggagcgcaaaactggaacaacgcagaaagactggttgaatttggggctcctcacgctgtcaagatggacgtgtacaacctcggagaccagactggagtgttactgaaggctctcgctggggttcctgtggcacacattgagggaaccaagtaccacctgaagagtggccacgtgacctgcgaagtgggactggaaaaactgaagatgaaaggtcttacgtacacaatgtgtgacaaaacaaagttcacatggaagagagctccaacagacagtgggcatgatacagtggtcatggaagtcacattctctggaacaaagccctgtaggatcccagtcagggcagtggcacatggatctccagatgtgaacgtggccatgctgataacgccaaacccaacaattgaaaacaatggaggtggcttcatagagatgcagctgcccccaggggataacatcatctatgttggggaactgagtcatcaatggttccaaaaagggagcagcatcggaagggttttccaaaagaccaagaaaggcatagaaagactgacagtgataggagagcacgcctgggacttcggttctgctggaggctttctgagttcaattgggaaggcggtacatacggtccttggtggcgctttcaacagcatcttcgggggagtggggtttctaccaaaacttttattaggagtggcattggcttggttgggcctgaacatgagaaaccctacaatgtccatgagctttctcttggctggaggtctggtcttggccatgacccttggagtgggggcggatgttggttgcgctgtggacacggaacgaatggagctccgctgtggcgagggcctggtcgtgtggagagaggtctcagaatggtatgacaattatgcctactacccggagacaccgggggcccttgcatcagccataaaggagacatttgaggagggaagctgtggtgtagtcccccagaacaggctcgagatggccatgtggagaagctcggtcacagagctgaatttggctctggcggaaggggaggcaaatctcacagtggtggtggacaagtttgaccccactgactaccgaggtggtgtccctggtttactgaaaaaaggaaaggacataaaagtctcctggaaaagctggggccattcaatgatctggagcattcctgaggccccccgtcgcttcatggtgggcacggaaggacaaagtgagtgtcccctagagagacggaagacaggtgttttcacggtggcagaattcggggttggcctgagaacaaaggtcttcttggatttcagacaggaaccaacacatgagtgtgacacaggagtgatgggagctgcagtcaagaacggcatggcaatccacacagatcaaagtctctggatgagatcaatgaaaaatgacacaggcacttacatagttgaacttttggtcactgacctgaggaactgctcatggcctgctagccacactatcgataatgctgacgtggtggactcagagttattccttccggcgagcctggcaggacccagatcctggtacaacaggatacctggctattcagaacaggtgaaagggccatggaagtacacgcctatccgagttatcagagaggagtgtcccggcacgaccgttaccatcaacgccaagtgtgacaaaagaggagcatctgtgaggagtaccacagagagtggcaaggttatcccagaatggtgctgccgagcgtgcacaatgccaccagtgacgttccggactggaactgattgctggtatgccatggaaatacggccagtccatgaccagggggggcttgttcgctcaatggtggttgcggacaacggtgaattacttagtgagggaggagtccccggaatagtggcattgtttgtggtccttgaatacatcatccgtaggaggccctccacgggaacgacggttgtgtgggggggtatcgtcgttctcgctctgcttgtcaccgggatggtcaggatagagagcctggtgcgctatgtggtggcagtggggatcacattccaccttgagctggggccagagatcgtggccttgatgctactccaggctgtgtttgagctgagggtgggtttgctcagcgcatttgcattgcgcagaagcctcaccgtccgagagatggtgaccacctactttcttttgctggtcctggaattggggctgccgggtgcgagccttgaggagttctggaaatggggtgatgcactggccatgggggcgctgatattcagggcttgcacggcagaaggaaagactggagcggggcttttgctcatggctctcatgacacagcaggatgtggtgactgtgcaccatggactggtgtgcttcctaagtgtagcttcggcatgctcggtctggaggctgctcaagggacacagagagcagaagggattgacctgggttgtccccctggctgggttgcttgggggagagggctctggaatcagactgctggcgttttgggagttgtcagcgcacagaggaagacgatctttcagtgaaccactaactgtggtaggagtcatgctaacactggccagcggcatgatgcgacacacttcccaggaggctctctgtgcactcgcagtggcctcgtttctcttgctgatgctggtgctggggacaagaaagatgcagctggttgccgaatggagtggctgcgttgaatggtatccggaactagtgaatgagggtggagaggttagcctgcgggtccggcaggacgcgatgggtaactttcacttgactgagctcgagaaagaagagagaatgatggctttttggctgattgccggcttggcagcttcggccattcactggtcaggcattcttggtgtgatgggactgtggacgctcacggaaatgctgaggtcatcccgaaggtctgacctggttttctctggacaggggggtcgagagcgtggtgacagacctttcgaggttaaggacggtgtctacaggatttttagccccggcttgttctggggtcagaaccaggtgggagttggctacggttccaagggtgtcttgcacacgatgtggcacgtgacgagaggagcggcgctgtctattgatgacgctgtggccggtccctactgggctgatgtgagggaagatgtcgtgtgttacggaggagcctggagcctggaggaaaaatggaaaggtgaaacagtacaggtccatgccttcccaccggggagggcgcatgaggtgcatcagtgccagcctggggagttgatccttgacaccggaagaaagcttggggcaataccaattgatttggtgaaaggaacatcaggcagccccattctcaacgcccagggagtggttgtggggctatacggaaatggcctaaaaactaatgagacctacgtcagcagcattgctcaaggggaagcggagaagagtcgccccaacctcccacaggctgttgtgggtacgggctggacatcaaagggtcagatcacagtgctggacatgcacccaggctcggggaagacccacagagtcctcccggagctcattcgccaatgcattgacaggcgcctgagaacgttggtgttggctccaactcgtgtggtactcaaagaaatggagcgtgctttgaatgggaaacgggtcaggttccactcaccagcagtcagtgaccaacaggctggaggggcaattgtcgatgtgatgtgtcacgcaacctatgtcaacagaaggctactcccacagggaagacaaaattgggaggtggcaatcatggatgaggcccactggacggacccccacagcatagctgccagaggtcatttgtatactctggcaaaagaaaacaagtgtgcactggtcttgatgacagcgacacctcctggtaagagtgaaccctttccggagtccaatggagccattactagtgaggaaagacagattcctgatggggagtggcgtgacgggtttgactggatcactgagtatgaagggcgcaccgcctggtttgtcccttcgattgcaaaaggtggtgctatagctcgcaccttgagacagaaggggaaaagtgtgatttgtttgaacagcaaaacctttgaaaaggactactccagagtgagggatgagaagcctgactttgtggtgacgactgatatctcggagatgggagctaaccttgacgtgagccgcgtcatagatgggaggacaaacatcaagcccgaggaggttgatgggaaagtcgagctcaccgggaccaggcgagtgaccacggcttccgctgcccaacggcgcggaagagttggtcggcaagacggacgaacagatgaatacatatactctggacagtgtgatgatgatgacagtggattagtgcaatggaaagaggcgcaaatacttcttgacaacataacaaccttgcgggggcccgtggccaccttctatggaccagaacaggacaagatgccggaggtggccggtcactttcgactcactgaagagaaaagaaagcacttccgacatcttctcacccattgtgacttcacaccgtggctggcatggcacgtcgcagcgaatgtatccagcgtcacggatcgaagctggacatgggaagggccggaggcaaatgccgtggatgaggccagtggtgacttggtcacctttaggagcccgaatggggcggagagaactctcaggccggtgtggaaggacgcacgtatgttcaaagagggacgtgacatcaaagagttcgtggcgtacgcgtctgggcgtcgcagcttcggagatgttctgacaggaatgtcgggagttcctgagctcctgcggcacagatgcgtcagtgccctggatgtcttctacacgcttatgcatgaggaacctggcagcagggcaatgagaatggcggagagagatgccccagaggcctttctgactatggttgagatgatggtgctgggtttggcaaccctgggtgtcatctggtgcttcgtcgtccggacttcaatcagccgcatgatgctgggcacgctggtcctgctggcctccttgctactcttgtgggcaggtggcgtcggctatgggaacatggctggagtggctctcatcttttacacgttgctgacggtgctgcagcctgaggcgggaaaacagagaagcagtgacgacaacaaactggcatatttcttgctgacgctctgcagccttgctggactggttgcagccaatgagatgggctttctggagaagaccaaggcagacttgtccacggcgctgtggagtgaacgggaggaaccccggccatggagtgaatggacgaatgtggacatccagccagcgaggtcctgggggacctatgtgctggtggtgtctctgttcacaccttacatcatccaccaactgcagaccaaaatccaacaacttgtcaacagtgccgtggcatctggtgcacaggccatgagagaccttgggggaggtgcccccttctttggtgtggcgggacatgtcatgaccctcggggtggtgtcactgattggggctactcccacctcactgatggtgggcgttggcttggcggcactccatctggccattgtggtgtctggtctggaggctgaattaacacagagagctcataaggtctttttctctgcaatggtgcgcaaccccatggtggatggggatgtcatcaacccattcggggagggggaggcaaaacctgctctatatgaaaggaaaatgagtctggtgttggccacagtgttgtgcctcatgtcggtggtcatgaaccgaacggtggcctccataacagaggcttcagcagtgggactggcagcagcgggacagctgcttagaccggaggctgacacgttgtggacgatgccggttgcttgtggcatgagtggtgtggtcaggggtagcctgtgggggtttttgcctcttgggcatagactctggcttcgagcctctgggggtaggcgtggtggttctgagggagacacgcttggagatctctggaagcggaggctgaacaactgcacgagggaggaattctttgtgtacaggcgcaccggcatcctggagacggaacgtgacaaggctagagagttgctcagaagaggagagaccaatgtgggattggctgtctctcggggcacggcaaagcttgcctggcttgaggaacgcggatatgccaccctcaagggagaggtggtagatcttggatgtggaaggggcggctggtcctattatgcggcatcccgaccggcagtcatgagtgtcagggcatataccattggtggaaaagggcacgaggctccaaagatggtaacaagcctgggttggaacttgattaaattcagatcaggaatggacgtgttcagcatgcagccacaccgggctgacactgtcatgtgtgacatcggagagagcagcccagatgccgctgtggagggtgagaggacaaggaaagtgatactgctcatggagcaatggaaaaacaggaaccccacggctgcctgtgtgttcaaggtgctggccccatatcgcccagaagtgatagaggcactgcacagattccaactgcaatggggggggggtctggtgaggacccctttttcaaggaactccacccatgagatgtattactcaacagccgtcactgggaacatagtgaactccgtcaatgtacagtcgaggaaacttttggctcggtttggagaccagagagggccaaccaaggtgcctgaactcgacctgggagttggaacgaggtgtgtggtcttagctgaggacaaggtgaaagaacaagacgtacaagagaggatcagagcgttgcgggagcaatacagcgaaacctggcatatggacgaggaacacccgtaccggacatggcagtactggggcagctaccgcacggcaccaaccggctcggcggcgtcactgatcaatggggttgtgaaacttctcagctggccatggaacgcacgggaagatgtggtgcgcatggctatgactgacacaacggctttcggacagcagagagtgttcaaagataaagttgacacaaaggcacaggagcctcagcccggtacaagagtcatcatgagagctgtaaatgattggattttggaacgactggcgcagaaaagcaaaccgcgcatgtgcagcagggaagaattcatagcaaaagtgaaatcaaatgcagccttgggagcttggtcagatgagcaaaacagatgggcaagtgcaagagaggctgtagaggatcctgcattctggcgcctcgtggatgaagagagagaaaggcacctcatggggagatgtgcgcactgcgtgtacaacatgatgggcaagagagaaaagaaactgggagagttcggagtggcgaaaggaagtcgggccatttggtacatgtggctggggagtcgctttttggagttcgaggctcttggattcttgaatgaagaccattgggcctctagagagtccagtggagctggagttgagggaataagcttgaactacctgggctggcacctcaagaagttgtcaaccctgaatggaggactcttctatgcagatgacacagctggctgggacacgaaagttaccaatgcagacttagaggatgaagaacagatcctacggtacatggagggtgagcacaaacaattggcaaccacaataatgcaaaaagcataccatgccaaagtcgtgaaggtcgcgaggccttcccgtgatggaggctgcatcatggatgtcatcacaagaagagaccaaagaggttcgggtcaggttgtgacctatgcccttaacaccctcaccaacataaaggtgcaattaatccgaatgatggaaggggaaggggtcatagaggcagcggatgcacacaacccgagactgcttcgagtggagcgctggctgaaagaacacggagaagagcgtcttggaagaatgctcgtcagtggtgacgattgtgtggtgaggcccttggatgacagatttggcaaagcactttactttctgaatgacatggccaagaccaggaaggacattggggaatgggagcactcagccggcttttcaagctgggaggaggtacccttttgttcacaccatttccacgagctagtgatgaaggatggacgcaccctggtggtgccgtgccgagaccaagatgaactcgttgggagggcgcgcatctcaccggggtgcggctggagtgtccgcgagacggcctgcctttcaaaagcctacgggcagatgtggctgctgagctacttccaccgacgagacctgaggacgctcgggcttgccattaactcagcagtgcctgccgattgggttcctaccggccgcacgacgtggagcattcatgccagtggggcctggatgaccacagaagacatgctggacgtttggaaccgggtgtggattctggacaaccctttcatgcagaacaaggaaagggtcatggagtggagggatgttccgtacctccctaaagctcaggacatgttatgttcctcccttgttgggaggagagaaagagcagaatgggccaagaacatctggggagcggtggaaaaggtgaggaagatgataggtcctgaaaagttcaaggactatctctcctgtatggaccgccatgacctgcactgggagctcagactggagagctcaataatctaaacccagactgtgacagagcaaaacccggaaggctcgtaaaagattgtccggaaccaaaagaaaagcaagcaactcacagagatagagctcggactggagagctctttaaacaaaaaaaaaaaaaaaaaaaaaaaaaaaaaaaaaaaaaaaaaaaaaaaaagccagaattgagctgaacctggagagctcattaaatacagtccagacgaaacaaaacatgacaaagcaaagaggctgagctaaaagttcccactacgggactgcttcatagcggtttgtggggggaggctaggaggcgaagccacagatcatggaatgatgcggcagcgcgcgagagcgacggggaagtggtcgcacccgacgcaccatccatgaagcaatacttcgtgagaccccccctgaccagcaaagggggcagaccggtcaggggtgaggaatgcccccagagtgcattacggcagcacgccagtgagagtggcgacgggaaaatggtcgatcccgacgtagggcactctgaaaaattttgtgagaccccctgcatcatgataaggccgaacatggtgcatgaaaggggaggcccccggaagcacgcttccgggaggagggaagagagaaattggcagctctcttcaggatttttcctcctcctatacaaaattccccctcggtagagggggggcggttcttgttctccctgagccaccatcacccagacacaggtagtctgacaaggaggtgatgtgtgactcggaaaaacacccgct

SEQ ID NO: 29 - DNA, 10962 bp, West Nile virus
agtagttcgcctgtgtgagctgacaaacttagtagtgtttgtgaggattaacaacaattaacacagtgcgagctgtttcttggcacgaagatctcgatgtctaagaaaccaggagggcccggtaaaaaccgggctgtcaatatgctaaaacgcggtatgccccgcggattgtccttgataggactaaagagggctatgctgagtctgattgacgggaagggcccaatacgtttcgtgttggctcttttggcgtttttcagattcactgcaatcgctccgactcgtgcggtgctggacagatggagaggcgtcaacaaacaaacagcaatgaagcatctcttgagtttcaagaaagaactaggaactctgaccagtgccatcaaccgccggagcacaaaacaaaagaaaagaggaggcacagcgggctttactatcttgcttgggctgatcgcctgtgctggagctgtgaccctctcgaacttccagggcaaagtgatgatgacagtcaatgcaaccgatgtcactgacgtgattaccattccaacagctgctgggaaaaacctgtgcatcgtaagggctatggacgtaggatacctttgtgaggatactatcacttatgaatgtccggtcctagctgctggaaatgaccctgaagacattgactgctggtgcacgaaatcatctgtttacgtgcgctatggaagatgcacaaaaactcggcattcccgtcgaagcagaaggtctctgacagtccagacacatggagaaagtacactggccaacaagaaaggagcttggttggacagcacaaaagccacgagatatctggtgaagacagaatcatggatactgagaaacccgggctacgccctcgttgcagctgtcattggatggatgctaggaagcaacacaatgcaacgcgtcgtgtttgccattctattgctcctggtggcaccagcatacagcttcaactgtttaggaatgagtaacagagacttcctggagggagtgtctggagctacatgggttgatctggtactggaaggcgatagttgtgtgaccataatgtcaaaagacaagccaaccattgatgtcaaaatgatgaacatggaagcagccaacctcgcagatgtgcgcagttactgttacctagcttcggtcagtgacttgtcaacaagagctgcgtgtccaaccatgggtgaagcccacaacgagaaaagagctgaccccgccttcgtttgcaagcaaggcgttgtggacagaggatggggaaatggctgcggactgtttggaaaggggagcattgacacatgtgcgaagtttgcctgtacaaccaaagcaactggatggatcatccagaaggaaaacatcaagtatgaggttgccatatttgtgcatggcccgacgaccgttgaatctcatggcaagataggggccacccaggctggaagattcagtataactccatcggcgccatcttacacgctaaagttgggtgagtatggtgaggttacggttgattgtgagccacggtcaggaatagacaccagcgcctattacgttatgtcagttggtgagaagtccttcctggttcaccgagaatggtttatggatctgaacctgccatggagcagtgctggaagcaccacgtggaggaaccgggaaacactgatggagtttgaagaacctcatgccaccaaacaatctgttgtggctctagggtcgcaggaaggtgcgttgcaccaagctctggccggagcgattcctgttgagttctcaagcaacactgtgaagttgacatcaggacatctgaagtgtcgggtgaagatggagaagttgcagctgaagggaacaacatatggagtatgttcaaaagcgttcaaattcgctaggactcccgctgacactggccacggaacggtggtgttggaactgcaatataccggaacagacggtccctgcaaagtgcccatttcttccgtagcttccctgaatgacctcacacctgttggaagactggtgaccgtgaatccatttgtgtctgtggccacagccaactcgaaggttttgattgaactcgaacccccgtttggtgactcttacatcgtggtgggaagaggagaacagcagataaaccatcactggcacaaatctgggagcagcattggaaaggcctttaccaccacactcagaggagctcaacgactcgcagctcttggagatactgcttgggattttggatcagttggaggggttttcacctcagtggggaaagccatacaccaagtctttggaggagcttttagatcactctttggagggatgtcctggatcacacagggacttctgggagctcttctgttgtggatgggaatcaatgcccgtgacaggtcaattgctatgacgtttcttgcggttggaggagttttgctcttcctttcggtcaacgtccatgctgacacaggctgtgccattgatattggcaggcaagagctccggtgcggaagtggagtgtttatccacaacgatgtggaagcctggatggatcgttacaagttctacccggagacgccacagggcctagcaaaaattatccagaaagcacatgcagaaggagtctgcggcttgcgttccgtttccagactcgagcaccaaatgtgggaagccattaaggatgagctgaacaccctgttgaaagagaatggagtcgacttgagtgtcgtggtggaaaaacagaatgggatgtacaaagcagcaccaaaacgtttggctgccaccaccgaaaaactggagatgggttggaaggcttggggcaagagtatcatctttgcgccagaactagctaacaacacctttgtcatcgacggtcctgagactgaggaatgcccaacggccaaccgagcatggaacagtatggaggtagaggactttggatttggactgacaagcactcgcatgttcctgaggattcgggaaacgaacacaacggaatgcgactcgaagatcataggaaccgccgtcaagaacaacatggctgtgcatagtgatctatcatactggatagagagcggactcaacgacacctggaagcttgagagggcggttctaggagaagtcaaatcatgcacctggccagaaacccacactctgtggggtgatggagttctggaaagtgatctcatcatacccatcaccttggcaggacccagaagcaaccacaacaggagaccagggtacaaaactcagaaccaaggcccatgggatgaggggcgcgtcgagattgactttgactattgcccaggaacaacagtaactataagtgacagttgcgaacaccgtggacctgcggcacgcacaaccactgagagtgggaagctcatcacagactggtgctgcagaagttgcaccctccctccactgcgcttccagactgagaatggctgttggtatggaatggaaattcgacctacgcggcacgacgaaaagaccctcgtgcaatcgagagtgaatgcatacaacgccgacatgattgatccttttcagttgggccttatggtcgtgttcttggccacccaggaggtccttcgcaagaggtggacggccaagatcagcattccagctatcatgcttgcactcctagtcctagtgtttgggggtattacgtacactgatgtcctgcgatatgtcattctcgtcggcgccgcgtttgctgaagcaaactcaggaggagacgtcgtgcacttggcacttatggctacattcaagattcaaccagtctttctggtggcttcctttttgaaggcaaggtggaccaaccaagagagtattttgctcatgcttgcagctgctttcttccaaatggcttactatgacgccaagaatgttctgtcatgggaagtgcctgacgttttgaactctctctccgttgcgtggatgattctcagagctataagcttcaccaacacttcaaatgtggtggtgccgctgctggcccttttgacacctggattgaaatgcttaaaccttgatgtgtacagaattttgctactcatggttggagttggaagcctcatcaaagaaaaaaggagctctgcagcaaaaaagaaaggagcttgcctcatctgcctagcgctggcgtctacaggagtgttcaatccaatgatacttgcagctgggctaatggcttgcgaccccaaccgcaagcggggctggcctgctacagaagtgatgactgcagttggactcatgtttgccatcgttgggggtctggcagaacttgacatagattctatggctatccccatgaccatcgccggacttatgttcgcggcatttgtcatctctggaaagtcaacagacatgtggattgagaggacggctgacattacttgggagagtgatgctgaaatcacaggctctagcgaaagagtagatgtgaggctggatgatgatggaaattttcaactgatgaatgaccccggggcaccatggaaaatttggatgcttaggatggcctgcctggcgataagtgcctacacaccttgggcaattctcccctcggtcatcggattctggataacccttcagtacacaaagagaggaggtgttctttgggacacaccatcacccaaggagtacaagaagggtgataccaccactggcgtttacagaatcatgactcgaggtctgcttggcagttaccaagctggagccggagtgatggtagagggggtgttccacacactatggcacaccactaagggagctgctctcatgagtggtgagggacgtctggatccctactgggggagcgtgaaagaggaccgactttgctatggggggccatggaaactccaacataaatggaatggacatgatgaggtccaaatgattgtcgtggagccagggaaaaatgtgaaaaacgtccagaccaagcccggagtgtttaagacaccagaaggagaaattggggcagttacgctagactatcctaccggaacgtcaggttcccccattgtagacaaaaatggagatgtgattggattgtatgggaacggcgtcatcatgcctaatggttcatacataagcgccattgtgcaaggagagagaatggaagaaccggcaccagctggcttcgaacctgaaatgttgaggaagaaacagatcactgtccttgatctgcaccccggagcaggaaagacacgcaagatacttccccaaatcatcaaggaggccatcaacaaaagattgaggacggctgtgctggcacccaccagggtcgttgctgctgagatgtctgaggccctgagaggacttcccattcggtaccaaacctcagcagtgcacagagagcacagtggaaatgagatcgttgatgtcatgtgccatgccaccctcacacacaggctgatgtctccacacagagtccccaactacaacctgttcataatggatgaagcccatttcacggatccagcgagcatcgcagccagaggatacatagcaaccaaggttgaattgggcgaagccgccgcgattttcatgacggcaacgccacccgggacttctgacccctttccagagtctaatgctcctatctcggacatgcaaacagagatcccagacagagcctggaacactggatatgaatggataactgagtatgttggaaagaccgtttggtttgttccaagtgtgaaaatgggaaatgagattgccctctgtctgcaacgggcggggaagaaggttatccagctgaacagaaagtcctatgagacagagtaccccaagtgtaagaacgatgattgggattttgtcatcaccacagacatatcagaaatgggagccaacttcaaggcgagcagagtgatcgacagccgcaaaagcgtgaaacccaccatcattgaggaaggtgatggaagagtcatcctgggggaaccctcagccatcacggctgccagcgctgctcagcggagaggacgcataggaagaaacccatcacaagttggtgatgagtattgctatggagggcacacaaatgaggatgattccaactttgctcactggacagaggctcgcatcatgctagacaacatcaacatgccgaatggtctggtggctcaactatatcagcctgagcgcgagaaggtgtacaccatggacggggaatacaggctcagaggggaagaacggaagaacttccttgaattcctgagaacagctgatttaccagtctggctcgcttacaaagtggcagcagcaggaatatcataccatgaccggaaatggtgctttgatggacctcgaaccaacacgattcttgaagacaacaatgaagttgaagtcatcacgaagttgggtgagagaaagatcctaagacccaggtgggcagatgctagagtgtactcagaccatcaagctctaaagtccttcaaagattttgcatcggggaaacgatcacaaatcgggctcgttgaggtgctcgggagaatgcctgaacacttcatggtgaaaacttgggaggcattggacacgatgtatgtggtggcgaccgctgaaaaaggaggccgagctcacaggatggctcttgaggagctaccggacgcccttcagacaatagttttgattgcactattgagtgtgatgtccttaggtgtgttttttctactcatgcaaaggaagggcattggtaagattggcttgggaggagtaatcttaggagctgccacattcttctgctggatggctgaagtcccaggaacgaaaatagcaggcatgctcctgctttccctgctgctcatgattgttttgattccggagccggaaaagcagcgctcacagactgataaccagctcgccgtgttcttgatctgtgtgctcacactggtcggcgccgtggctgccaatgaaatgggctggctggacaagaccaagaatgacattggcagcctgttggggcacaggccagaagctagagagacgaccctgggagttgagagcttcttacttgatctgcggccggccacggcatggtcgctctatgccgtaacgacagccgttctcacccctttgctgaagcatctaatcacgtcagactacatcaacacttcgttgacctcaataaacgtccaagccagcgcgttgttcactttggccagaggcttcccttttgtggacgttggtgtgtcagctctcttgctggcggtcgggtgctggggtcaggtgactctgactgtgactgtgactgcagctgctctgctcttttgccactatgcttacatggtgccaggctggcaagcggaagccatgcgatctgcccagcggcggacagctgctggcatcatgaaaaatgtagtggtggatgggatcgtggccactgatgtacctgaacttgaacgaacaactccagtcatgcagaaaaaagttggacagatcatattgatcttggtatcaatggccgcggtggtcgtcaatccatcagtgagaaccgtcagagaggccggaattctgactacagcagcagcagtcaccctatgggagaatggtgctagttcagtgtggaatgcaacgacagctattggcctttgtcacatcatgcgaggaggatggctctcgtgtctctccatcatgtggactctcatcaaaaacatggagaaaccaggcctcaagaggggtggagccaaaggacgcacgctaggggaagtttggaaggagagactcaaccacatgacgaaggaagaatttaccagatacagaaaagaagccatcactgaagttgaccgctccgcagcaaaacatgctaggagagagggaaacatcactggaggccacccagtctcacggggaaccgcgaaattacggtggttagtggaaaggcgtttcctcgagccagtgggaaaggttgtggatctcgggtgtggtagaggcggctggtgctattacatggctacccagaagagggtacaggaagtgaaagggtacacgaaaggaggacctggccatgaagaaccacaactggtgcagagctatggttggaatattgttaccatgaagagtggagtcgacgtcttctacagaccatcagaagcgagcgacacactgctctgtgacattggagagtcatcgtcaagtgccgaggtagaagaacaccgcaccgtccgtgtcctggagatggtggaagattggttgcacagaggaccgaaggaattctgcatcaaagtgctatgcccttacatgcccaaagtgattgagaagatggaaacactccaaaggcgatatggaggtggccttataagaaaccccctttcacgcaactctacccatgagatgtactgggtgagccacgcttcaggcaatatcgtccactccgtcaacatgacaagccaggtgcttctggggaggatggaaaagaaaacatggaagggaccccagtttgaggaagatgtcaacttgggaagtggaacgcgggcagtagggaagcctctcctcaattctgatactagcaagatcaagaaccgaattgagaggctgaagaaagaatacagctccacatggcaccaggatgcgaaccacccctacaggacctggaactaccacggaagctatgaagtgaaaccaaccggctcagccagctcccttgtgaatggggtagtcagattactctcaaaaccatgggacactatcaccaatgtgaccacgatggccatgacagacaccactcctttcggtcaacaacgagtgttcaaggaaaaggtggacacaaaggctccagagcctccagaaggagtcaaatacgtcctcaatgagaccacgaactggctgtgggcttttttagcccgcgataagaaacccaggatgtgttcccgggaggaatttattggaaaagtcaacagtaatgccgccctaggagcgatgtttgaagaacagaaccaatggaagaacgcccgggaagctgtagaggatccaaagttttgggagatggtggatgaggagcgtgaagcgcatctccgtggagaatgcaacacctgcatctacaacatgatgggaaagagagagaagaagcctggagagttcggcaaagctaaaggcagcagagccatctggttcatgtggctgggggcccgcttcctggagtttgaagctctcggattcctcaatgaagaccactggctgggtaggaagaactcaggaggaggagttgaaggcttaggactgcagaagctcgggtacatcttgaaggaagttggaacaaagcctggaggaaaggtttacgctgatgataccgcaggctgggacacacgcatcaccaaagctgacctcgagaatgaagcgaaggttcttgaactgctggatggagaacatcgacgtttagcgcggtccatcatcgagctcacataccgacacaaagtcgtgaaagtgatgaggccagcggccgacgggaaaactgtgatggacgtcatctctagagaggatcagagaggaagcggtcaggtagtgacttacgccctgaacaccttcaccaatctagcagttcagctggtcagaatgatggagggggagggggtcattggacccgatgatgttgaaaaactgggaaaaggaaaaggccctaaggtcagaacctggctgtttgagaatggcgaggagcgtctcagtcgcatggccgtcagcggtgatgactgcgtggtgaaacctttggacgaccgcttcgccacatcactacacttcctaaatgctatgtcaaaggtccgcaaagacatccaggaatggaaaccctcgacggggtggtatgactggcagcaggttccattctgttcaaaccatttcacggaactgatcatgaaggacggcaggacgctggtggtcccgtgtcgtggacaagacgagttgattggacgtgccaggatctctccaggggctggatggaatgtgcgcgacaccgcctgcctggcgaagtcatacgcgcagatgtggctgctgctttatttccaccgtagagacctgagattgatggccaatgccatctgttccgctgtgcctgccaactgggttcccacagggcgtaccacttggtcgatccacgcaaaaggagaatggatgacgacggaagacatgctcgcagtctggaacagagtgtggattgaggagaatgagtggatggaagacaaaacaccagttgagaggtggagtgatgttccatactctggaaagagagaggacatttggtgtggcagtttgatcggcacacgaacccgcgccacttgggctgaaaatatccatgtggcaatcaatcaggtccgttcagtgattggagaagagaagtatgtggattacatgagctccttgaggaggtatgaagacaccattgtagtggaggacactgttttgtaaaagatagtattatagttagtttagtgtaaataggatttattgagaatggaagtcaggccagattaatgctgccaccggaagttgagtagacggtgctgcctgcggctcaaccccaggaggactgggtgaccaaagctgcgaggtgatccacgtaagccctcagaaccgtctcggaaggaggaccccacgtgctttagcctcaaagcccagtgtcagaccacactttaatgtgccactctgcggagagtgcagtctgcgatagtgccccaggtggactgggttaacaaaggcaaaacatcgccccacgcggccataaccctggctatggtgttaaccagggagaagggactagaggttagaggagaccccgcgtaaaaaagtgcacggcccaacttggctgaagctgtaagccaagggaaggactagaggttagaggagaccccgtgccaaaaacaccaaaagaaacagcatattgacacctgggatagactaggggatcttctgctctgcacaaccagccacacggcacagtgcgccgacataggtggctggtggtgctagaacacaggatct

SEQ ID NO: 30 - DNA, 10807 bp, Flavivirus FSME
agttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgacgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagctgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagtaacttagtaaggtcaatggtgactgcaggatcaactgatcacatggatcacttctcccttggagtgcttgtgattctgctcatggtgcaggaagggctgaagaagagaatgaccacaaagatcatcataagcacatcgatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagcggcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttagtgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 31 - DNA, 10862 bp, Yellow fever virus
agtaaatcctgtgtgctaattgaggtgcattggtctgcaaatcgagttgctaggcaataaacacatttggattaattttaatcgttcgttgagcgattagcagagaactgaccagaacatgtctggtcgtaaagctcagggaaaaaccctgggcgtcaatatggtacgacgaggagttcgctccttgtcaaacaaaataaaacaaaaaacaaaacaaattggaaacagacctggaccttcaagaggtgttcaaggatttatctttttctttttgttcaacattttgactggaaaaaagatcacagcccacctaaagaggttgtggaaaatgctggacccaagacaaggcttggctgttctaaggaaagtcaagagagtggtggccagtttgatgagaggattgtcctcaaggaaacgccgttcccatgatgttctgactgtgcaattcctaattttgggaatgctgttgatgacgggtggagtgaccttggtgcggaaaaacagatggttgctcctaaatgtgacatctgaggacctcgggaaaacattctctgtgggcacaggcaactgcacaacaaacattttggaagccaagtactggtgcccagactcaatggaatacaactgtcccaatctcagtccaagagaggagccagatgacattgattgctggtgctatggggtggaaaacgttagagtcgcatatggtaagtgtgactcagcaggcaggtctaggaggtcaagaagggccattgacttgcctacgcatgaaaaccatggtttgaagacccggcaagaaaaatggatgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctcttttgaagaagggaagtgtggcctaaattcagttgactcccttgagcatgagatgtggagaagcagggcagatgagatcaatgccatttttgaggaaaacgaggtggacatttctgttgtcgtgcaggatccaaagaatgtttaccagagaggaactcatccattttccagaattcgggatggtctgcagtatggttggaagacttggggtaagaaccttgtgttctccccagggaggaagaatggaagcttcatcatagatggaaagtccaggaaagaatgcccgttttcaaaccgggtctggaattctttccagatagaggagtttgggacgggagtgttcaccacacgcgtgtacatggacgcagtctttgaatacaccatagactgcgatggatctatcttgggtgcagcggtgaacggaaaaaagagtgcccatggctctccaacattttggatgggaagtcatgaagtaaatgggacatggatgatccacaccttggaggcattagattacaaggagtgtgagtggccactgacacatacgattggaacatcagttgaagagagtgaaatgttcatgccgagatcaatcggaggcccagttagctctcacaatcatatccctggatacaaggttcagacgaacggaccttggatgcaggtaccactagaagtgaagagagaagcttgcccagggactagcgtgatcattgatggcaactgtgatggacggggaaaatcaaccagatccaccacggatagcgggaaagttattcctgaatggtgttgccgctcctgcacaatgccgcctgtgagcttccatggtagtgatgggtgttggtatcccatggaaattaggccaaggaaaacgcatgaaagccatctggtgcgctcctgggttacagctggagaaatacatgctgtcccttttggtttggtgagcatgatgatagcaatggaagtggtcctaaggaaaagacagggaccaaagcaaatgttggttggaggagtagtgctcttgggagcaatgctggtcgggcaagtaactctccttgatttgctgaaactcacagtggctgtgggattgcatttccatgagatgaacaatggaggagacgccatgtatatggcgttgattgctgccttttcaatcagaccagggctgctcatcggctttgggctcaggaccctatggagccctcgggaacgccttgtgctgaccctaggagcagccatggtggagattgccttgggtggcgtgatgggcggcctgtggaagtatctaaatgcagtttctctctgcatcctgacaataaatgctgttgcttctaggaaagcatcaaataccatcttgcccctcatggctctgttgacacctgtcactatggctgaggtgagacttgccgcaatgttcttttgtgccgtggttatcataggggtccttcaccagaatttcaaggacacctccatgcagaagactatacctctggtggccctcacactcacatcttacctgggcttgacacaaccttttttgggcctgtgtgcatttctggcaacccgcatatttgggcgaaggagtatcccagtgaatgaggcactcgcagcagctggtctagtgggagtgctggcaggactggcttttcaggagatggagaacttccttggtccgattgcagttggaggactcctgatgatgctggttagcgtggctgggagggtggatgggctagagctcaagaagcttggtgaagtttcatgggaagaggaggcggagatcagcgggagttccgcccgctatgatgtggcactcagtgaacaaggggagttcaagctgctttctgaagagaaagtgccatgggaccaggttgtgatgacctcgctggccttggttggggctgccctccatccatttgctcttctgctggtccttgctgggtggctgtttcatgtcaggggagctaggagaagtggggatgtcttgtgggatattcccactcctaagatcatcgaggaatgtgaacatctggaggatgggatttatggcatattccagtcaaccttcttgggggcctcccagcgaggagtgggagtggcacagggaggggtgttccacacaatgtggcatgtcacaagaggagctttccttgtcaggaatggcaagaagttgattccatcttgggcttcagtaaaggaagaccttgtcgcctatggtggctcatggaagttggaaggcagatgggatggagaggaagaggtccagttgatcgcggctgttccaggaaagaacgtggtcaacgtccagacaaaaccgagcttgttcaaagtgaggaatgggggagaaatcggggctgtcgctcttgactatccgagtggcacttcaggatctcctattgttaacaggaacggagaggtgattgggctgtacggcaatggcatccttgtcggtgacaactccttcgtgtccgccatatcccagactgaggtgaaggaagaaggaaaggaggagctccaagagatcccgacaatgctaaagaaaggaatgacaactgtccttgattttcatcctggagctgggaagacaagacgtttcctcccacagatcttggccgagtgcgcacggagacgcttgcgcactcttgtgttggcccccaccagggttgttctttctgaaatgaaggaggcttttcacggcctggacgtgaaattccacacacaggctttttccgctcacggcagcgggagagaagtcattgatgctatgtgccatgccaccctaacttacaggatgttggaaccaactagggttgttaactgggaagtgatcattatggatgaagcccattttttggatccagctagcatagccgctagaggttgggcagcgcacagagctagggcaaatgaaagtgcaacaatcttgatgacagccacaccgcctgggactagtgatgaatttccacattcaaatggtgaaatagaagatgttcaaacggacatacccagtgagccctggaacacagggcatgactggatcctggctgacaaaaggcccacggcatggttccttccatccatcagagctgcaaatgtcatggctgcctctttgcgtaaggctggaaagagtgtggtggtcctgaacaggaaaacctttgagagagaataccccacgataaagcagaagaaacctgactttatattggccactgacatagctgaaatgggagccaacctttgcgtggagcgagtgctggattgcaggacggcttttaagcctgtgcttgtggatgaagggaggaaggtggcaataaaagggccacttcgtatctccgcatcctctgctgctcaaaggagggggcgcattgggagaaatcccaacagagatggagactcatactactattctgagcctacaagtgaaaataatgcccaccacgtctgctggttggaggcctcaatgctcttggacaacatggaggtgaggggtggaatggtcgccccactctatggcgttgaaggaactaaaacaccagtttcccctggtgaaatgagactgagggatgaccagaggaaagtcttcagagaactagtgaggaattgtgacctgcccgtttggctttcgtggcaagtggccaaggctggtttgaagacgaatgatcgtaagtggtgttttgaaggccctgaggaacatgagatcttgaatgacagcggtgaaacagtgaagtgcagggctcctggaggagcaaagaagcctctgcgcccaaggtggtgtgatgaaagggtgtcatctgaccagagtgcgctgtctgaatttattaagtttgctgaaggtaggaggggagctgctgaagtgctagttgtgctgagtgaactccctgatttcctggctaaaaaaggtggagaggcaatggataccatcagtgtgtttctccactctgaggaaggctctagggcttaccgcaatgcactatcaatgatgcctgaggcaatgacaatagtcatgctgtttatactggctggactactgacatcgggaatggtcatctttttcatgtctcccaaaggcatcagtagaatgtctatggcgatgggcacaatggccggctgtggatatctcatgttccttggaggcgtcaaacccactcacatctcctatatcatgctcatattctttgtcctgatggtggttgtgatccccgagccagggcaacaaaggtccatccaagacaaccaagtggcatacctcattattggcatcctgacgctggtttcagcggtggcagccaacgagctaggcatgctggagaaaaccaaagaggacctctttgggaagaagaacttaattccatctagtgcttcaccctggagttggccggatcttgacctgaagccaggagctgcctggacagtgtacgttggcattgttacaatgctctctccaatgttgcaccactggatcaaagtcgaatatggcaacctgtctctgtctggaatagcccagtcagcctcagtcctttctttcatggacaaggggataccattcatgaagatgaatatctcggtcataatgctgctggtcagtggctggaattcaataacagtgatgcctctgctctgtggcatagggtgcgccatgctccactggtctctcattttacctggaatcaaagcgcagcagtcaaagcttgcacagagaagggtgttccatggcgttgccaagaaccctgtggttgatgggaatccaacagttgacattgaggaagctcctgaaatgcctgccctttatgagaagaaactggctctatatctccttcttgctctcagcctagcttctgttgccatgtgcagaacgcccttttcattggctgaaggcattgtcctagcatcagctgccctagggccgctcatagagggaaacaccagccttctttggaatggacccatggctgtctccatgacaggagtcatgagggggaatcactatgcttttgtgggagtcatgtacaatctatggaagatgaaaactggacgccgggggagcgcgaatggaaaaactttgggtgaagtctggaagagggaactgaatctgttggacaagcgacagtttgagttgtataaaaggaccgacattgtggaggtggatcgtgatacggcacgcaggcatttggccgaagggaaggtggacaccggggtggcggtctccagggggaccgcaaagttaaggtggttccatgagcgtggctatgtcaagctggaaggtagggtgattgacctggggtgtggccgcggaggctggtgttactacgctgctgcgcaaaaggaagtgagtggggtcaaaggatttactcttggaagagacggccatgagaaacccatgaatgtgcaaagtctgggatggaacatcatcaccttcaaggacaaaactgatatccaccgcctagaaccagtgaaatgtgacacccttttgtgtgacattggagagtcatcatcgtcatcggtcacagagggggaaaggaccgtgagagttcttgatactgtagaaaaatggctggcttgtggggttgacaacttctgtgtgaaggtgttagctccatacatgccagatgttctcgagaaactggaattgctccaaaggaggtttggcggaacagtgatcaggaaccctctctccaggaattccactcatgaaatgtactacgtgtctggagcccgcagcaatgtcacatttactgtgaaccaaacatcccgcctcctgatgaggagaatgaggcgtccaactggaaaagtgaccctggaggctgacgtcatcctcccaattgggacacgcagtgttgagacagacaagggacccctggacaaagaggccatagaagaaagggttgagaggataaaatctgagtacatgacctcttggttttatgacaatgacaacccctacaggacctggcactactgtggctcctatgtcacaaaaacctcaggaagtgcggcgagcatggtaaatggtgttattaaaattctgacatatccatgggacaggatagaggaggtcacaagaatggcaatgactgacacaaccccttttggacagcaaagagtgtttaaagaaaaagttgacaccagagcaaaggatccaccagcgggaactaggaagatcatgaaagttgtcaacaggtggctgttccgccacctggccagagaaaagaaccccagactgtgcacaaaggaagaatttattgcaaaagtccgaagtcatgcagccattggagcttacctggaagaacaagaacagtggaagactgccaatgaggctgtccaagacccaaagttctgggaactggtggatgaagaaaggaagctgcaccaacaaggcaggtgtcggacttgtgtgtacaacatgatggggaaaagagagaagaagctgtcagagtttgggaaagcaaagggaagccgtgccatatggtatatgtggctgggagcgcggtatcttgagtttgaggccctgggattcctgaatgaggaccattgggcttccagggaaaactcaggaggaggagtggaaggcattggcttacaatacctaggatatgtgatcagagacctggctgcaatggatggtggtggattctacgcggatgacaccgctggatgggacacgcgcatcacagaggcagaccttgatgatgaacaggagatcttgaactacatgagcccacatcacaaaaaactggcacaagcagtgatggaaatgacatacaagaacaaagtggtgaaagtgttgagaccagccccaggagggaaagcctacatggatgtcataagtcgacgagaccagagaggatccgggcaggtagtgacttatgctctgaacaccatcaccaacttgaaagtccaattgatcagaatggcagaagcagagatggtgatacatcaccaacatgttcaagattgtgatgaatcagttctgaccaggctggaggcatggctcactgagcacggatgtaacagactgaagaggatggcggtgagtggagacgactgtgtggtccggcccatcgatgacaggttcggcctggccctgtcccatctcaacgccatgtccaaggttagaaaggacatatctgaatggcagccatcaaaagggtggaatgattgggagaatgtgcccttctgttcccaccacttccatgaactacagctgaaggatggcaggaggattgtggtgccttgccgagaacaggacgagctcattgggagaggaagggtgtctccaggaaacggctggatgatcaaggaaacagcttgcctcagcaaagcctatgccaacatgtggtcactgatgtattttcacaaaagggacatgaggctactgtcattggctgtttcctcagctgttcccacctcatgggttccacaaggacgcacaacatggtcgattcatgggaaaggggagtggatgaccacggaagacatgcttgaggtgtggaacagagtatggataaccaacaacccacacatgcaggacaagacaatggtgaaaaaatggagagatgtcccttatctaaccaagagacaagacaagctgtgcggatcactgattggaatgaccaatagggccacctgggcctcccacatccatttggtcatccatcgtatccgaacgctgattggacaggagaaatacactgactacctaacagtcatggacaggtattctgtggatgctgacctgcaactgggtgagcttatctgaaacaccatctaacaggaataaccgggatacaaaccacgggtggagaaccggactccccacaacctgaaaccgggatataaaccacggctggagaaccggactccgcacttaaaatgaaacagaaaccgggataaaaactacggatggagaaccggactccacacattgagacagaagaagttgtcagcccagaaccccacacgagttttgccactgctaagctgtgaggcagtgcaggctgggacagccgacctccaggttgcgaaaaacctggtttctgggacctcccaccccagagtaaaaagaacggagcctccgctaccaccctcccacgtggtggtagaaagacggggtctagaggttagaggagaccctccagggaacaaatagtgggaccatattgacgccagggaaagaccggagtggttctctgcttttcctccagaggtctgtgagcacagtttgctcaagaataagcagacctttggatgacaaacacaaaaccact

SEQ ID NO: 32 - DNA, 755 bp, Artificial Sequence
  Synthetic
gagttaatttttaaaaagcagtcaaaagtccaagtggcccttgcgagcatttactctctctgtttgctctggttaataatctcaggagcacaaaattccttactagtcctagaagttaatttttaaaaagcagtcaaaagtccaagtccaagtggcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaatgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaaggttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatcagcttacatgt

SEQ ID NO: 33 - DNA, 3162 bp, Staphylococcus aureus
atgaaaaggaattatatcttaggattagatatcggaattacatcagtgggttatggaattattgattatgaaactagagatgtcatagatgcgggcgtacgtttatttaaagaggctaatgttgaaaataatgaaggacgacgatcaaaaagaggtgccagaaggcttaagaggcgtcgtagacatagaatacaaagagtaaagaaacttttatttgattacaatttgttgacagatcatagtgagctaagtggaatcaatccttacgaggcgcgcgtaaagggattaagtcaaaaattaagtgaagaggaattttctgcggcattgctacatttagcaaagcgtagaggtgtacataatgttaatgaagtggaagaagatacaggtaatgaattatccactaaagaacaaatttcaagaaatagtaaagcgttagaagagaagtatgttgcagaattacagttggaacgtttgaaaaaagacggtgaagtgagaggttcgattaaccgtttcaaaacatctgactatgtaaaagaagcaaagcagttattaaaagtacaaaaagcatatcatcaacttgatcaatcatttatagacacttatattgatttattggaaacaagaagaacatattatgagggaccaggtgaaggtagcccatttggatggaaagatattaaagaatggtatgaaatgttaatgggacattgtacgtatttcccagaagaattacgtagtgtgaaatatgcctataatgctgatttatataatgcgctgaatgatttgaacaacttggttattacacgagatgagaatgagaagctagagtattatgaaaaattccaaattatcgagaatgtctttaaacaaaagaaaaagccgacgcttaaacaaattgcgaaggaaatcttggtgaatgaagaagacatcaaaggctatcgtgtcacaagtacaggtaaaccagaatttacaaacttgaaagtttatcacgatatcaaagatattacagcaagaaaagaaattatcgagaatgcagagctactcgatcaaatagctaaaatattaactatttaccagtcatcagaagatatacaagaagaattaacaaacctaaattcagaattgacacaagaagagattgaacaaatttcaaacttgaaaggttatacaggaactcataacctttcactaaaggcaataaatttaatattagacgaattgtggcatacgaacgataatcaaatagctattttcaatcgtttgaaacttgtacctaaaaaggtagatttaagccaacaaaaagaaattcctactactttagttgatgattttatactgtctccagtagtgaaacgttcatttatacaatctattaaagttattaacgctattattaaaaaatacggtttgccaaatgatattattattgaacttgcgagagaaaagaattctaaagatgcacaaaaaatgattaatgaaatgcagaagagaaatcgtcaaacgaatgaacgtattgaggaaattataagaacgacaggtaaagaaaatgctaaatatttaattgaaaaaattaagctgcacgatatgcaagaagggaaatgtttatactcgttagaagcaatccctctagaagatttacttaataatccattcaattacgaagtagaccatatcattccacgttctgtttctttcgataactctttcaataataaagtgttggtgaaacaagaagaaaatagtaaaaaaggtaatagaacgccatttcaatatttaagttcttcagattctaaaataagttatgagacattcaaaaagcatattttaaatcttgctaaaggcaaaggtagaatctctaagacgaaaaaagaatatttgttagaagaacgagatatcaatcgcttcagtgtccaaaaagattttattaaccgtaacttagtagatacacgctatgcgacaagagggttaatgaacttattaagatcttattttagagtgaataacttagatgtcaaagtgaaatcgattaatggcggattcacaagtttcttaagaaggaaatggaagttcaaaaaagaaagaaataagggctacaaacaccatgctgaagatgcactgattattgcgaacgctgattttattttcaaagaatggaaaaaactagataaagctaaaaaagtgatggaaaatcaaatgtttgaagaaaagcaagctgaaagtatgcctgaaattgagactgagcaagagtataaagaaatttttataacgcctcatcaaattaaacatattaaggattttaaagattataaatattcacatagagttgataaaaagccgaatagagagttaataaatgatacattatattctacgagaaaagatgacaagggtaatacattaatcgttaataacttaaatggtttatacgataaagataatgataaattgaaaaaattaattaataaatcacctgaaaaattattgatgtatcatcatgatccacaaacatatcaaaaattaaaattgatcatggaacaatatggcgatgagaaaaatccgctttataaatattatgaagaaacaggcaattacttaacaaaatatagtaaaaaagataacggaccagtcatcaaaaaaattaaatattatggtaacaagctaaatgcgcatttagatattacggatgattatccaaatagcagaaataaagtagtaaaactttcattaaaaccatatcgctttgatgtttatttagataatggggtatataaatttgtgacagttaaaaatttagatgttatcaaaaaagaaaactactatgaagttaattcaaagtgttatgaagaagcaaaaaaactgaagaaaattagtaatcaagcagaatttatcgcaagtttttacaataatgacttgattaagattaacggagaattatatagagtcataggtgtaaataatgatctacttaacagaattgaagtaaatatgatagacatcacatatagagaatatttagagaacatgaatgataaaagaccacctagaataattaaaacaatagcaagcaaaacacaatctattaaaaagtattctacagatattctaggcaatctttatgaagtgaagagtaaaaagcatcctcaaatcataaagaaaggatga

SEQ ID NO: 34 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 35 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGLTIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 36 - DNA, 1865 bp, Gallus gallus
ttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 37 - DNA, 311 bp, Dengue virus type 2
gctagcaggcaaaactaacatgaaacaaggcaaaaaatctgggaggccacaaaccatggaagctgtacgcatggcgtagtggactagcggttagaggagacccctcccttacaaatcgcagcaacaacgggggcccaaggtgagatgaagctgtagtctcactggaaggactagaggttagaggagacccccccaaaacaaaaaacagcatattgacgctgggaaagaccagagatcctgctgtctcctcagcatcattccaggcacagaacgccagaaaatggaatggtgctgttgaatcaacaggttct

SEQ ID NO: 38 - DNA, 303 bp, Dengue virus type 2
gctagcaggcaaaactaacatgaaacaaggctaaaagtcaggtcggatcaagccatagtacggaaaaaactatgctacctgtgagccccgtccaaggacgttaaaagaagtcaggccatcacaaatgccacagcttgagtaaactgtgcagcctgtagctccacctgagaaggtgtaaaaaatctgaaaacaaaaaacagcatattgacgctgggaaagaccagagatcctgctgtctcctcagcatcattccaggcacagaacgccagaaaatggaatggtgctgttgaatcaacaggttct

SEQ ID NO: 39 - DNA, 363 bp, Dengue virus type 2
gctagcaggcaaaactaacatgaaacaaggctaaaagtcaggtcggatcaagccatagtacggaaaaaactatgctacctgtgagccccgtccaaggacgttaaaagaagtcaggccatcacaaatgccacagcttgagtaaactgtgcagcctgtagctccacctgagaaggtgtaaaaaatctgggaggccacaaaccatggaagctgtacgcatggcgtagtggactagcggttagaggagacccctcccttacaaatcgcagcaacaacgggggcccaaggtgagatgaagctgtagtctcactggaaggactagaggttagaggagacccccccaaaacaaaaaacagcatattgacg

(19) United States
(12) Patent Application Publication
(10) Pub. No.: US 2024/0131197 A1
(43) Pub. Date: Apr. 25, 2024

(54) Compositions For and Methods of Improving Gene Therapy

(71) Applicant: Duke University, Durham, NC (US)

(72) Inventors: Aravind Asokan, Durham, NC (US); Rita Meganck, Durham, NC (US)

(21) Appl. No.: 18/547,332

(22) PCT Filed: Feb. 24, 2022
(86) PCT No.: PCT/US2022/017647

§ 371 Date: Aug. 20, 2023

Related U.S. Application Data
(60) Provisional application No. 63/153,985, filed on Feb. 26, 2021.

(51) Int. Cl. A61K 48/00; C12N 15/86

(52) U.S. Cl. CPC A61K 48/0066; C12N 15/86; C12N 2750/14143; C12N 2770/24122; C12N 2770/24143

(57) ABSTRACT

Disclosed herein are compositions comprising one or more subgenomic Flavivirus RNA (sfRNA) elements and using those compositions in methods of improving mRNA transcript stability, improving mRNA transcript translation efficiency, enhancing gene therapy, and treating and/or preventing a disease or disorder.

### I. CROSS-REFERENCE TO RELATED APPLICATIONS

This application claims priority to U.S. Provisional Application No. 63/153,985 filed 26 Feb. 2021, which is incorporated herein in its entirety.

### II. REFERENCE TO THE SEQUENCE LISTING

The Sequence Listing submitted 25 Feb. 2022 as a text file named “21_2023_WO_Sequence_Listing”, created on 25 Feb. 2022 and having a size of 208 kilobytes is hereby incorporated by reference pursuant to 37 C.F.R. § 1.52(e)(5).

### III. BACKGROUND

Gene transfer using recombinant adeno-associated virus (rAAV) vectors has been successfully used to treat hemophilia, inherited vision loss, and spinal muscular atrophy. The development of new rAAV vectors for the treatment of a wide range of genetic diseases, such as neurological disorders and muscular dystrophies, is advancing at an unprecedented rate. However, suboptimal transgene expression using recombinant AAV vectors is a significant challenge facing the field of gene therapy. This has necessitated the use of very high viral doses in clinical trials, leading to toxic side effects and even some patient deaths. Thus, new strategies to increase transgene expression are needed to enhance AAV gene therapy efficacy and allow for lower, safer dosing regimens.

Currently, there are no disease-modifying therapies for the many, many genetic diseases and disorders that plague the human population. Thus, there remains an urgent need for a minimally invasive, definitive therapy to address the underlying cause of as well as the sequelae of symptoms associated with these various genetic diseases and disorders. Consequently, the present disclosure provides compositions for and methods of improving mRNA transcript stability, improving mRNA transcript translation efficiency, enhancing therapy, and treating and/or preventing a disease and/or disorder, which can be used alone or in combination with other treatments.

### IV. BRIEF DESCRIPTION OF THE FIGURES

FIG. 1A shows the secondary structure of the DENV2 sfRNA including 2 xrRNAs, 2 DBs, and a 3′ SL. Various structural elements are noted. CS1/3′CYC indicates conserved sequence 1 with the 3′ cyclization sequence. CS2 indicates conserved sequence 2 while CS3 indicates conserved sequence. FIG. 1B shows the sequence conservation of the xrRNA element of subgenomic Flaviviral RNAs for DENV 1 and DENV 2, JEV 1 and JEV 2, MVEV 1 and MVEV 2, WNV2 1 and WNV 2, YFV, and ZIKV 1 and ZIKV 2. FIG. 1C shows the sequence conservation of the DB element of subgenomic Flaviviral RNAs for DENV, JEV, MVEV, WNV2, YFV, and ZKV. FIG. 1D show the sequence conservation of the 3′ SL of subgenomic Flaviviral RNAs for DENV2, JEV, MVEV, WNV2, YFV, and ZIKV.

FIG. 2A-FIG. 2B show that XRN1 restricts AAV transduction. FIG. 2A shows Huh7 cells following infection with lentivirus packaging Cas9 and either a Scramble gRNA (top) or gRNA against the XRN1 gene (bottom). Following antibiotic selection, sequencing at the XRN1 locus confirmed Cas9 activity with the XRN1-1 gRNA. The arrow denotes the predicted Cas9 cleavage site. FIG. 2B shows western blotting performed on two independent Scramble and XRN1 KO clonal cell lines for XRN1 (top) and R-actin (bottom). FIG. 2C shows the quantitation of transgene expression from different AAV serotypes in XRN1 KO cells.

FIG. 3A-FIG. 3E show that subgenomic Flaviviral RNAs increased AAV transduction efficiency. FIG. 3A shows a diagram of an experimental construct. Here, a luciferase transgene with SV40 poly-A signal and driven by the CBA promoter was placed between AAV2 inverted terminal repeats (ITRs). SfRNA (or WPRE) sequences were placed as the 3′ UTR of the luciferase mRNA. FIG. 3B shows luciferase expression in Huh7 cells transduced with the indicated constructs at 10,000 vg/cell and harvested at 3 days post-transduction. Lysates were measured for Relative Luciferase Expression (RLUs). FIG. 3C shows RNA from replicate samples was used to perform qRT-PCR for luciferase mRNAs. FIG. 3D shows mRNA expression in various cell lines were transduced with 10,000 vg/cell of the DENV2 sfRNA construct and harvested at 3 days post-transduction. Lysates were measured for Relative Luciferase Expression (RLUs). FIG. 3E shows RNA from replicate samples was used to perform qRT-PCR for luciferase mRNAs. For all graphs, samples are plotted relative to a CBA-Luc transgene with no additional 3′ UTR. Student's t-test was performed to test for statistical significance. Where indicated for FIG. 3B-FIG. 3E, * =p<0.05; ** =p<0.005; *** =p<0.0005; **** =p<0.00005.

FIG. 4A-FIG. 4C shows that the effect of DENV2 sfRNA on AAV transduction was context-dependent. FIG. 4A shows a construct in which the DENV2 sfRNA was placed in the 5′ of the transgene (top construct), 3′ of the transgene (middle construct), or as a separate and U6-driven RNA packaged in the same AAV genome (lower construct). FIG. 4B shows northern blots of HEK293 cells transfected with the various constructs in FIG. 4A and then harvested 3 days post-transfection. The northern blots were probed for either luciferase or sfRNA sequences. FIG. 4C-FIG. 4D show Huh7 cells transduced with the indicated constructs at 10,000 vg/cell and then harvested 3 days post-transfection. FIG. 4C shows lysates that were measured for Relative Luciferase Expression (RLUs) while FIG. 4D shows RNA from replicate samples that was used to perform qRT-PCR for luciferase mRNAs. Data was relative to a CBA-Luc transgene with no additional 3′ UTR. For FIG. 4C-FIG. 4D, Student's t-test was performed to test for statistical significance, and where indicated, * =p<0.05; ** =p<0.005; *** =p<0.0005; **** =p<0.00005.

FIG. 5A-FIG. 5E shows that the presence of polyA tail was necessary for increased transgene expression. FIG. 5A shows a diagram of luciferase mRNAs with either a poly-A or RiboJ (hammerhead ribozyme) 3′ end with and without the DENV2 sfRNA. FIG. 5B shows northern blots of HEK293 cells following transfection with the indicated constructs and then harvested 3 days post-transfection. The northern blots were probed for luciferase sequences. FIG. 5C shows Huh7 cells transduced with the indicated constructs at 10,000 vg/cell and then harvested at 3 days post-transduction. Lysates were measured for Relative Luciferase Expression (RLUs). FIG. 5D shows qRT-PCR for luciferase mRNAs using replicate samples. Data is shown relative to a CBA-Luc transgene with no additional 3′ UTR. FIG. 5E shows the ratio of protein and RNA expression calculated for the DENV2 RiboJ construct relative to CBA-Luc with a poly-A tail. Student's t-test was performed to test for statistical significance. In FIG. 5C-FIG. 5E, where indicated, * =p<0.05; ** =p<0.005; *** =p<0.0005; **** =p<0.00005.

FIG. 6A-FIG. 6F shows that the DENV2 DB elements were necessary and sufficient to increase AAV transduction efficiency. FIG. 6A shows a diagram of the DENV2 sfRNA with the indicated deletions of RNA structural elements. FIG. 6B shows Huh7 cells transduced with the indicated constructs at 10,000 vg/cell and harvested at 3 days post-transduction. Lysates were measured for Relative Luciferase Expression (RLUs). FIG. 6C shows qRT-PCR for luciferase mRNAs performed on replicate samples. Data is shown relative to a CBA-Luc transgene with no additional 3′ UTR. FIG. 6D shows mutations in the first DB of DENV2 that were created at the indicated locations. Huh7 cells were transduced with the indicated constructs at 10,000 vg/cell and harvested at 3 days post-transduction. FIG. 6E shows Relative Luciferase Expression (RLUs) from lysates and FIG. 6F shows qRT-PCR for luciferase mRNAs performed on replicate samples. Data is shown relative to a CBA-Luc transgene with no additional 3′ UTR. Student's t-test was performed to test for statistical significance. In FIG. 6B-FIG. 6C and FIG. 6E-FIG. 6F, where indicated, * =p<0.05; ** =p<0.005; *** =p<0.0005; **** =p<0.00005.

FIG. 7A-FIG. 7D show that DENV2 sfRNA and DB elements increase mRNA half-life. HEK293 cells were transduced with the indicated constructs at 10,000 vg/cell. At 3 days post-transduction, Actinomycin D was added to cells and RNA collected at the indicated time points. RNA levels were measured by qRT-PCR, and graphed relative to the level at 0 hours and normalized to GAPDH mRNA levels. FIG. 7A shows the fraction RNA remaining for 3 constructs. FIG. 7B shows that the DENV2 DB increased the mRNA half-life while FIG. 7C shows that the DENV2 sfRNA also increased the mRNA half-life. FIG. 7D shows that DENV2 sfRNA and DENV2 DB generated RNA having longer half-lives. Student's t-test was performed to test for statistical significance. In FIG. 7A-FIG. 7D, where indicated, * =p<0.05; ** =p<0.005; *** =p<0.0005; **** =p<0.00005.

### V. BRIEF SUMMARY

Disclosed herein is an isolated nucleic acid molecule, comprising: one or more Flavivirus genetic elements. Disclosed herein is an isolated nucleic acid molecule, comprising one or more subgenomic Flavivirus RNA (sfRNA) elements.

Disclosed herein is an isolated nucleic acid molecule, comprising one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is an isolated nucleic acid molecule, comprising a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is an isolated nucleic acid molecule, comprising one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is an isolated nucleic acid molecule, comprising a nucleic acid sequence encoding a polypeptide or an RNA molecule, and a nucleic acid sequence encoding one or more Flavivirus genetic elements. Disclosed herein is an isolated nucleic acid molecule, comprising a nucleic acid sequence encoding a polypeptide or an RNA molecule, and a nucleic acid sequence encoding one or more subgenomic Flavivirus RNA (sfRNA) elements.

Disclosed herein is an isolated nucleic acid molecule, comprising a nucleic acid sequence encoding a polypeptide or an RNA molecule, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is an expression cassette, comprising a nucleic acid sequence encoding a polypeptide or an RNA molecule operably linked to a promoter, and a nucleic acid sequence encoding one or more Flavivirus genetic elements.

Disclosed herein is an expression cassette, comprising a nucleic acid sequence encoding a polypeptide or an RNA molecule operably linked to a promoter, and a nucleic acid sequence encoding one or more subgenomic Flavivirus RNA (sfRNA) elements.

Disclosed herein is an expression cassette, comprising a nucleic acid sequence encoding a polypeptide or an RNA molecule operably linked to a promoter, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is a vector, comprising a disclosed isolated nucleic acid molecule. Disclosed herein is a vector, comprising an isolated nucleic acid molecule, comprising a nucleic acid sequence encoding a polypeptide or an RNA molecule, and a nucleic acid sequence encoding one or more Flavivirus genetic elements. Disclosed herein is a vector, comprising an isolated nucleic acid molecule, comprising a nucleic acid sequence encoding a polypeptide or an RNA molecule, and a nucleic acid sequence encoding one or more subgenomic Flavivirus RNA (sfRNA) elements.

Disclosed herein is a vector, comprising an isolated nucleic acid molecule, comprising a nucleic acid sequence encoding a polypeptide or an RNA molecule, and a nucleic acid sequence encoding a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is a vector, comprising an isolated nucleic acid molecule, comprising a nucleic acid sequence encoding a polypeptide or an RNA molecule, and a nucleic acid sequence encoding one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is a pharmaceutical formulation comprising an isolated nucleic acid molecule, comprising one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is a pharmaceutical formulation comprising an isolated nucleic acid molecule, comprising a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding one or more subgenomic Flavivirus RNA (sfRNA) elements.

Disclosed herein is a pharmaceutical formulation comprising a disclosed vector comprising a nucleic acid sequence encoding a polypeptide or an RNA molecule, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is a method of improving mRNA transcript stability, comprising administering to a subject a therapeutically effective amount of an isolated nucleic acid molecule encoding a transgene and encoding a Flavivirus 3′ untranslated region (3′ UTR) element, wherein expression of the nucleic acid molecule improves mRNA transcript stability.

Disclosed herein is a method of improving mRNA transcript stability, comprising administering to a subject a therapeutically effective amount of an isolated nucleic acid molecule encoding a transgene and encoding one or more Flavivirus genetic elements, wherein expression of the nucleic acid molecule improves mRNA transcript stability.

Disclosed herein is a method of improving mRNA transcript stability, comprising administering to a subject a therapeutically effective amount of an isolated nucleic acid molecule encoding a transgene and encoding one or more subgenomic Flavivirus RNA (sfRNA) elements, wherein expression of the nucleic acid molecule improves mRNA transcript stability.

Disclosed herein is a method of improving mRNA transcript stability, comprising administering to a subject a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript stability.

Disclosed herein is a method of improving mRNA transcript stability, comprising administering to a subject a therapeutically effective amount of a vector comprising an isolated nucleic acid molecule encoding a transgene and encoding one or more Flavivirus genetic elements, wherein expression of the nucleic acid molecule improves mRNA transcript stability.

Disclosed herein is a method of improving mRNA transcript stability, comprising administering to a subject a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript stability.

Disclosed herein is a method of improving the mRNA transcript translation efficiency, comprising administering to a subject a therapeutically effective amount of an isolated nucleic acid molecule encoding a transgene and encoding a Flavivirus 3′ untranslated region (3′ UTR) element, wherein expression of the nucleic acid molecule improves mRNA transcript translation efficiency.

Disclosed herein is a method of improving the mRNA transcript translation efficiency, comprising administering to a subject a therapeutically effective amount of an isolated nucleic acid molecule encoding a transgene and encoding one or more Flavivirus genetic elements, wherein expression of the nucleic acid molecule improves mRNA transcript translation efficiency.

Disclosed herein is a method of improving the mRNA transcript translation efficiency, comprising administering to a subject a therapeutically effective amount of an isolated nucleic acid molecule encoding a transgene and encoding one or more subgenomic Flavivirus RNA (sfRNA) elements, wherein expression of the nucleic acid molecule improves mRNA transcript translation efficiency.

Disclosed herein is a method of improving the mRNA transcript translation efficiency, comprising administering to a subject a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript translation efficiency.

Disclosed herein is a method of improving the mRNA transcript translation efficiency, comprising administering to a subject a therapeutically effective amount of a vector comprising an isolated nucleic acid molecule encoding a transgene and encoding one or more Flavivirus genetic elements, wherein expression of the nucleic acid molecule improves mRNA transcript translation efficiency.

Disclosed herein is a method of improving the mRNA transcript translation efficiency, comprising administering to a subject a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript translation efficiency.

Disclosed herein is a method of enhancing gene therapy, comprising administering to a subject in need thereof a therapeutically effective amount of an isolated nucleic acid molecule encoding a transgene and encoding a Flavivirus 3′ untranslated region (3′ UTR) element, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of an isolated nucleic acid molecule encoding a transgene and encoding one or more Flavivirus genetic elements, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of an isolated nucleic acid molecule encoding a transgene and encoding one or more subgenomic Flavivirus RNA (sfRNA) elements, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of a vector comprising an isolated nucleic acid molecule encoding a transgene and encoding a Flavivirus 3′ untranslated region (3′ UTR) element, wherein expression of the nucleic acid molecule improves transgene mRNA stability and/or improves mRNA transcript translation efficiency.

Disclosed herein is a method of enhancing gene therapy, comprising administering to a subject in need thereof a therapeutically effective amount of a vector comprising an isolated nucleic acid molecule encoding a transgene and encoding one or more Flavivirus genetic elements, wherein expression of the nucleic acid molecule improves transgene mRNA stability and/or improves mRNA transcript translation efficiency.

Disclosed herein is a method of enhancing gene therapy, comprising administering to a subject in need thereof a therapeutically effective amount of a vector comprising an isolated nucleic acid molecule encoding a transgene and encoding one or more subgenomic Flavivirus RNA (sfRNA) elements, wherein expression of the nucleic acid molecule improves transgene mRNA stability and/or improves mRNA transcript translation efficiency.

Disclosed herein is a method of treating and/or preventing a disease or disorder, comprising enhancing gene therapy by administering to a subject in need thereof a therapeutically effective amount of an isolated nucleic acid molecule encoding a transgene and encoding a Flavivirus 3′ untranslated region (3′ UTR) element, wherein expression of the nucleic acid molecule improves transgene mRNA stability and/or improves mRNA transcript translation efficiency.

Disclosed herein is a method of treating and/or preventing a disease or disorder, comprising enhancing gene therapy by administering to a subject in need thereof a therapeutically effective amount of an isolated nucleic acid molecule encoding a transgene and encoding one or more Flavivirus genetic elements, wherein expression of the nucleic acid molecule improves transgene mRNA stability and/or improves mRNA transcript translation efficiency.

Disclosed herein is a method of treating and/or preventing a disease or disorder, comprising enhancing gene therapy by administering to a subject in need thereof a therapeutically effective amount of an isolated nucleic acid molecule encoding a transgene and encoding one or more subgenomic Flavivirus RNA (sfRNA) elements, wherein expression of the nucleic acid molecule improves transgene mRNA stability and/or improves mRNA transcript translation efficiency.

Disclosed herein is a method of treating and/or preventing a disease or disorder, comprising administering to a subject in need thereof a therapeutically effective amount of an isolated nucleic acid molecule encoding a transgene and encoding a Flavivirus 3′ untranslated region (3′ UTR) element, and enhancing gene therapy through improved mRNA transcript mRNA stability and/or improved mRNA transcript translation efficiency.

Disclosed herein is a method of treating and/or preventing a disease or disorder, comprising administering to a subject in need thereof a therapeutically effective amount of an isolated nucleic acid molecule encoding a transgene and encoding one or more Flavivirus genetic elements, and enhancing gene therapy through improved mRNA transcript mRNA stability and/or improved mRNA transcript translation efficiency.

Disclosed herein is a method of treating and/or preventing a disease or disorder, comprising administering to a subject in need thereof a therapeutically effective amount of an isolated nucleic acid molecule encoding a transgene and encoding one or more subgenomic Flavivirus RNA (sfRNA) elements, and enhancing gene therapy through improved transgene mRNA stability and/or improved mRNA transcript translation efficiency.

Disclosed herein is a method of treating and/or preventing a disease or disorder, comprising enhancing gene therapy by administering to a subject in need thereof a therapeutically effective amount of a vector comprising an isolated nucleic acid molecule encoding a transgene and encoding a Flavivirus 3′ untranslated region (3′ UTR) element, wherein expression of the nucleic acid molecule improves transgene mRNA stability and/or improves mRNA transcript translation efficiency.

Disclosed herein is a method of treating and/or preventing a disease or disorder, comprising enhancing gene therapy by administering to a subject in need thereof a therapeutically effective amount of a vector comprising an isolated nucleic acid molecule encoding a transgene and encoding one or more Flavivirus genetic elements, wherein expression of the nucleic acid molecule improves transgene mRNA stability and/or improves mRNA transcript translation efficiency.

Disclosed herein is a method of treating and/or preventing a disease or disorder, comprising enhancing gene therapy by administering to a subject in need thereof a therapeutically effective amount of a vector comprising an isolated nucleic acid molecule encoding a transgene and encoding one or more subgenomic Flavivirus RNA (sfRNA) elements, wherein expression of the nucleic acid molecule improves transgene mRNA stability and/or improves mRNA transcript translation efficiency.

Disclosed herein is a method of treating and/or preventing a disease or disorder, comprising administering to a subject in need thereof a therapeutically effective amount of a vector comprising an isolated nucleic acid molecule encoding a transgene and encoding a Flavivirus 3′ untranslated region (3′ UTR) element, and enhancing gene therapy through improved transgene mRNA stability and/or improved mRNA transcript translation efficiency.

Disclosed herein is a method of treating and/or preventing a disease or disorder, comprising administering to a subject in need thereof a therapeutically effective amount a vector comprising of an isolated nucleic acid molecule encoding a transgene and encoding one or more Flavivirus genetic elements, and enhancing gene therapy through improved transgene mRNA stability and/or improved mRNA transcript translation efficiency.

Disclosed herein is a method of treating and/or preventing a disease or disorder, comprising administering to a subject in need thereof a therapeutically effective amount of a vector comprising an isolated nucleic acid molecule encoding a transgene and encoding one or more subgenomic Flavivirus RNA (sfRNA) elements, and enhancing gene therapy through improved transgene mRNA stability and/or improved mRNA transcript translation efficiency.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method.

The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, “derived from” can mean “derived from”, “based on”, “obtained from”, “obtained from” or “isolated from” depending on the context. For example, in an aspect, the term “derived from” can mean that an amino acid sequence is derived from the parent amino acid sequence by introducing a modification to at least one position. Thus, the derived amino acid sequence differs from the corresponding parent amino acid sequence in at least one corresponding position (numbering based on Kabat's EU index numbering system for the antibody Fc region). In an aspect, a disclosed amino acid sequence derived from the parent amino acid sequence can differ by 1 to 15 amino acid residues at corresponding positions. In an aspect, a derived amino acid sequence can have a high degree sequence identity to its parent amino acid sequence. In an aspect, a disclosed amino acid sequence derived from the parent amino acid sequence can have at least 50%, at least 60%, at least 70%, at least 80%, at least 90%, or more sequence identity to the parent or original sequence.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease or disorder, be suspected of having a disease or disorder, or be at risk of developing a disease or disorder (e.g., a genetic disease or disorder).

As used herein, the term “diagnosed” means having been subjected to an examination by a person of skill, for example, a physician, and found to have a condition that can be diagnosed or treated by one or more of the disclosed isolated nucleic acid molecules, disclosed vectors, disclosed pharmaceutical formulations, or a combination thereof, or by one or more of the disclosed methods. For example, “diagnosed with a disease or disorder” means having been subjected to an examination by a person of skill, for example, a physician, and found to have a condition (such as a genetic disease or disorder) that can be treated by one or more of the disclosed isolated nucleic acid molecules, disclosed vectors, disclosed pharmaceutical formulations, or a combination thereof, or by one or more of the disclosed methods. For example, “suspected of having a disease or disorder” can mean having been subjected to an examination by a person of skill, for example, a physician, and found to have a condition (such as a genetic disease or disorder) that can likely be treated by one or more of by one or more of the disclosed isolated nucleic acid molecules, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a disease or disorder (e.g., a genetic disease or disorder). In an aspect, a patient can refer to a subject that has been diagnosed with or is suspected of having a disease or disorder. In an aspect, a patient can refer to a subject that has been diagnosed with or is suspected of having a disease or disorder and is seeking treatment or receiving treatment for a disease or disorder.

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., a genetic disease or disorder) based upon an earlier diagnosis by a person of skill and thereafter subjected to treatment for the genetic disease or disorder. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder such as a genetic disease or disorder). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having a genetic disease or disorder). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as a genetic disease or disorder). For example, treating a disease or disorder can reduce one or more symptoms of a disease or disorder in a subject by 1%-100% as compared to a control (such as, for example, an individual not having a genetic disease or disorder). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder. It is understood that treatment does not necessarily refer to a cure or complete ablation or eradication of a disease or disorder. However, in an aspect, treatment can refer to a cure or complete ablation or eradication of a disease or disorder.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as a genetic disease or disorder) a or related complication from progressing to that complication.

As used herein, the terms “administering” and “administration” refer to any method of providing one or more of the disclosed isolated nucleic acid molecules, disclosed vectors, disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed isolated nucleic acid molecule, a disclosed vector, a disclosed pharmaceutical composition, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route.

In an aspect, the skilled person can determine an efficacious dose, an efficacious schedule, and an efficacious route of administration for one or more of the disclosed isolated nucleic acid molecules, disclosed vectors, disclosed pharmaceutical formulations, or a combination thereof to treat or prevent a disease or disorder (such as genetic disease or disorder). In an aspect, the skilled person can also alter, change, or modify an aspect of an administering step to improve efficacy of one or more of the disclosed isolated nucleic acid molecules, disclosed vectors, disclosed pharmaceutical formulations, or a combination thereof.

By “determining the amount” is meant both an absolute quantification of a particular analyte (e.g., an mRNA sequence containing a particular tag) or a determination of the relative abundance of a particular analyte (e.g., an amount as compared to a mRNA sequence including a different tag). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, by changing the duration of time one or more of the disclosed isolated nucleic acid molecules, disclosed vectors, disclosed pharmaceutical formulations, or a combination are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

The term “contacting” as used herein refers to bringing one or more of the disclosed isolated nucleic acid molecules, disclosed vectors, disclosed pharmaceutical formulations, or a combination thereof together with a target area or intended target area in such a manner that the one or more of the disclosed isolated nucleic acid molecules, disclosed vectors, disclosed pharmaceutical formulations, or a combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by a disease or disorder (such as a genetic disease or disorder). In an aspect, a target area or intended target area can be any organ, tissue, or cells that are affected by a disease or disorder (such as a genetic disease or disorder).

As used herein, “determining” can refer to measuring or ascertaining the presence and severity of a disease or disorder, such as, for example, a genetic disease or disorder. Methods and techniques used to determine the presence and/or severity of a disease or disorder are typically known to the medical arts. For example, the art is familiar with the ways to identify and/or diagnose the presence, severity, or both of a disease or disorder (such as, for example, a genetic disease or disorder).

As used herein, “effective amount” and “amount effective” can refer to an amount that is sufficient to achieve the desired result such as, for example, the treatment and/or prevention of a disease or disorder (e.g., a genetic disease or disorder) or a suspected disease or disorder. As used herein, the terms “effective amount” and “amount effective” can refer to an amount that is sufficient to achieve the desired an effect on an undesired condition (e.g., a disease or disorder). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or disorder (e.g., a genetic disease or disorder), (ii) attenuates, ameliorates, or eliminates one or more symptoms of the particular disease, condition, or disorder e.g., a genetic disease or disorder), or (iii) delays the onset of one or more symptoms of the particular disease, condition, or disorder described herein (e.g., a genetic disease or disorder). The specific therapeutically effective dose level for any particular patient will depend upon a variety of factors including the disorder being treated and the severity of the disorder; the disclosed isolated nucleic acid molecules, disclosed vectors, disclosed pharmaceutical formulations employed; the disclosed methods employed; the age, body weight, general health, sex and diet of the patient; the time of administration; the route of administration; the rate of excretion of the disclosed isolated nucleic acid molecules, disclosed vectors, or disclosed pharmaceutical formulations employed; the duration of the treatment; drugs used in combination or coincidental with the disclosed isolated nucleic acid molecules, disclosed vectors, or disclosed pharmaceutical formulations employed, and other like factors well known in the medical arts. For example, it is well within the skill of the art to start doses of the disclosed isolated nucleic acid molecules, disclosed vectors, or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder due to a missing, deficient, and/or mutant protein or enzyme.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, “lipid nanoparticles” or “LNPs” can deliver nucleic acid (e.g., DNA or RNA), protein (e.g., RNA-guided DNA binding agent), or nucleic acid together with protein. LNPs can comprise biodegradable, ionizable lipids. For example, LNPs can comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. In an aspect, the term cationic and ionizable in the context of LNP lipids can be use interchangeably, e.g., wherein ionizable lipids are cationic depending on the pH.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition in a disclosed method.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Liver-specific promoters” are known to the art and include, but are not limited to, the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter. In an aspect, a liver specific promoter can comprise about 845-bp and comprise the thyroid hormone-binding globulin promoter sequences (2382 to 13), two copies of enhancer sequences (22,804 through 22,704), and a 71-bp leader sequence as described by Ill C R, et al. (1997). In an aspect, a disclosed liver specific promoter can comprise the sequence set forth in SEQ ID NO:32, or a sequence having about 50%, about 60%, about 70% about 80%, about 90%, about 95%, or more identity to the sequence set forth in SEQ ID NO:32.

Ubiquitous/constitutive promoters” are known to the art and include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, the term “serotype” is a distinction used to refer to an AAV having a capsid that is serologically distinct from other AAV serotypes. Serologic distinctiveness can be determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “CRISPR or clustered regularly interspaced short palindromic repeat” is an ideal tool for correction of genetic abnormalities as the system can be designed to target genomic DNA directly. A CRISPR system involves two main components—a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Staphylococcus Aureus (SEQ ID NO:33 (nucleic acid) or SEQ ID NO:34 (amino acid) or Streptococcus pyogenes (SEQ ID NO:35).

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease can be a wild type CRISPR/Cas protein (such as for example, Staphylococcus Aureus (SEQ ID NO:33 (nucleic acid) or SEQ ID NO:34 (amino acid) or Streptococcus pyogenes (SEQ ID NO:35), a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein or an effector domain fused with a CRISPR/Cas protein.

In an aspect, a disclosed CRISPR-based endonuclease can be derived from a wild type Cas9 protein (such as, for example, SEQ ID NO:33) or fragment thereof. In an aspect, a disclosed CRISPR-based endonuclease can be derived from a modified Cas9 protein. For example, the amino acid sequence of a disclosed Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with a viral vector (such as, for example, AAV). Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

In an aspect, a “self-complementary parvovirus genome” can be a single stranded polynucleotide having, in the 5′ to 3′ direction, a first parvovirus ITR sequence, a heterologous sequence (e.g., payload construct comprising, for example, a desired gene), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a disease or disorder (such as a genetic disease or disorder).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions for Use in the Disclosed Methods

### 1. Nucleic Acid Molecules

Disclosed herein is an isolated nucleic acid molecule, comprising a nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is an isolated nucleic acid molecule, comprising a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is an isolated nucleic acid molecule, comprising: one or more Flavivirus genetic elements.

In an aspect, disclosed Flavivirus genetic elements can comprise a 3′ untranslated (3′ UTR) element, a subgenomic Flavivirus RNA (sfRNA) element, a Flavivirus XRN1-resistant RNA (xrRNA) element, a Flavivirus sfRNA dumbbell (DB) RNA element, a Flavivirus 3′ stem loop (3′ SL) element, or any combination thereof.

In an aspect, a disclosed Flavivirus genetic element can comprise a 3′ UTR element. For example, in an aspect, a disclosed 3′ UTR element can comprise the sequence set forth in any one of SEQ ID NO:08-SEQ ID NO:19. In an aspect, a disclosed 3′ UTR element comprises the sequence forth in SEQ ID NO:08 or a fragment thereof, SEQ ID NO:09 or a fragment thereof, SEQ ID NO:10 or a fragment thereof, SEQ ID NO:11 or a fragment thereof, SEQ ID NO:12 or a fragment thereof, SEQ ID NO:13 or a fragment thereof, SEQ ID NO:14 or a fragment thereof, SEQ ID NO:15 or a fragment thereof, SEQ ID NO:16 or a fragment thereof, SEQ ID NO:17 or a fragment thereof, SEQ ID NO:18 or a fragment thereof, SEQ ID NO:19 or a fragment thereof.

In an aspect, a disclosed 3′ UTR element can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in any one of SEQ ID NO:08-SEQ ID NO:19. In an aspect, a disclosed 3′ UTR element can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:08, SEQ ID NO:09, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19.

In an aspect, a disclosed Flavivirus genetic element can comprise a 3′ UTR element. For example, in an aspect, a disclosed 3′ UTR element can comprise the sequence set forth in any one of GenBank Accession Nos. U88535.1, KU725663.1, JQ411814.1, KJ160504.1, M55506.1, NC000943.1, KU886216.1, NC007580.2, U27495.1, M12294.2, KJ776791.2, MT107250.1. In an aspect, a disclosed 3′ UTR element can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in any one of GenBank Accession Nos. U88535.1, KU725663.1, JQ411814.1, KJ160504.1, M55506.1, NC000943.1, KU886216.1, NC007580.2, U27495.1, M12294.2, KJ776791.2, MT107250.1. In an aspect, a disclosed 3′ UTR element can comprise a sequence having at least 40%-60%, at least 60%-80%, at least 80%-90%, or at least 90%-100^{%}identity to the sequence set forth in GenBank Accession Nos. U88535.1, KU725663.1, JQ411814.1, KJ160504.1, M55506.1, NC000943.1, KU886216.1, NC007580.2, U27495.1, M12294.2, KJ776791.2, MT107250.1.

In an aspect, a disclosed 3′ UTR element can comprise a sequence having at least 40%-60%, at least 60%-80%, at least 80%-90%, or at least 90%-100^{%} identity to the sequence set forth in any one of SEQ ID NO:08-SEQ ID NO: 19. In an aspect, a disclosed 3′ UTR element can comprise a sequence having at least 40%-60%, at least 60%-80%, at least 80%-90%, or at least 90%-100% identity to the sequence set forth in SEQ ID NO:09, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19.

In an aspect, a disclosed 3′ UTR can comprise the following sequence or a fragment thereof:

(SEQ ID NO: 08)
GCCAACTCATTCACAAAATAAAGGAAAATAAAAAATCAAACAAGGCAAG
AAGTCAGGCCGGATTAAGCCATAGCACGGTAAGAGCTATGCTGCCTGTG
AGCCCCGTCCAAGGACGTAAAATGAAGTCAGGCCGAAAGCCACGGTTCG
AGCAAGCCGTGCTGCCTGTAGCTCCATCGTGGGGATGTAAAAACCCGGG
AGGCTGCAAACCATGGAAGCTGTACGCATGGGGTAGCAGACTAGTGGTT
AGAGGAGACCCCTCCCAAGACACAACGCAGCAGCGGGGCCCAACACCAG
GGGAAGCTGTACCCTGGTGGTAAGGACTAGAGGTTAGAGGAGACCCCCC
GCACAACAACAAACAGCATATTGACGCTGGGAGAGACCAGAGATCCTGC
TGTCTCTACAGCATCATTCCAGGCACAGAACGCCAAAAAATGGAATGGT
GCTGTTGAATCAACAGGTTCT.

In an aspect, a disclosed 3′ UTR can comprise the following sequence or a fragment thereof:

(SEQ ID NO: 09)
AAAGCAAAACTAACATGAAACAAGGCTAGAAGTCAGGTCGGATTAAGCC
ATAGTACGGAAAAAACTATGCTACCTGTGAGCCCCGTCCAAGGACGTTA
AAAGAAGTCAGGCCATCATAAATGCCATAGCTGGAGTAAACTATGCAGC
CTGTAGCTCCACCTGAGAAGGTGTAAAAAATCCGGGAGGCCACAAACCA
TGGAAGCTGTACGCATGGCGTAGTGGACTAGCGGTTAGAGGAGACCCCT
CCCTTACAAATCGCAGCAACAATGGGGGCCCAAGGCGAGATGAAGCTGT
AGTCTCGCTGGAAGGACTAGAGGTTAGAGGAGACCCCCCCGAAACAAAA
AACAGCATATTGACGCTGGGAAAGACCAGAGATCCTGCTGTCTCCTCAG
CATCATTCCAGGCACAGAACGCCAGAAAATGGAATGGTGCTGTTGAATC
AACAGGTTCT.

In an aspect, a disclosed 3′ UTR can comprise the following sequence or a fragment thereof:

(SEQ ID NO: 10)
ACGTAGGAAGTGAAAAAGAGGCAAACTGTCAGGCCACCTTAAGCCACAG
TACGGAAGAAGCTGTGCTGCCTGTGAGCCCCGTCCAAGGACGTTAAAAG
AAGAAGTCAGGCCCCAAAGCCACGGTTTGAGCAAACCGTGCTGCCTGTA
GCTCCGTCGTGGGGACGTAAAACCTGGGAGGCTGCAAACTGTGGAAGCT
GTACGCACGGTGTAGCAGACTAGCGGTTAGAGGAGACCCCTCCCATGAC
ACAACGCAGCAGCGGGGCCCGAGCACTGAGGGAAGCTGTACCTCCTTGC
AAAGGACTAGAGGTTAGAGGAGACCCCCCGCAAACAAAAACAGCATATT
GACGCTGGGAGAGACCAGAGATCCTGCTGTCTCCTCAGCATCATTCCAG
GCACAGAACGCCAGAAAATGGAATGGTGCTGTTGAATCAACAGGTTCT.

In an aspect, a disclosed 3′ UTR can comprise the following sequence or a fragment thereof:

(SEQ ID NO: 11)
TTACCAACAACAAACACCAAAGGCTATTGAAGTCAGGCCACTTGTGCCA
CGGTTTGAGCAAACCGTGCTGCCTGTAGCTCCGCCAACAATGGGAGGCG
TAATAATCCCCAGGGAGGCCATGCGCCACGGAAGCTGTACGCGTGGCAT
ATTGGACTAGCGGTTAGAGGAGACCCCTCCCATCACTGACAAAACGCAG
CAAAAAGGGGGCCCGAAGCCAGGAGGAAGCTGTACTCCTGGTGGAAGGA
CTAGAGGTTAGAGGAGACCCCCCCAACACAAAAACAGCATATTGACGCT
GGGAAAGACCAGAGATCCTGCTGTCTCTACAACATCAATCCAGGCACAG
AGCGCCGCAAGATGGATTGGTGTTGTTGATCCAACAGGTTCT.

In an aspect, a disclosed 3′ UTR can comprise the following sequence or a fragment thereof:

(SEQ ID NO: 12)
TGTGATTTAAGGTAGAAAAGTAGACTATGTAAATAATGTAAATGAGAAA
ATGCATGCATATGGAGTCAGGCCAGCAAAAGCTGCCACCGGATACTGGG
TAGACGGTGCTGCCTGCGTCTCAGTCCCAGGAGGACTGGGTTAACAAAT
CTGACAACAGAAAGTGAGAAAGCCCTCAGAACCGTCTCGGAAGTAGGTC
CCTGCTCACTGGAAGTTGAAAGACCAACGTCAGGCCACAAATTTGTGCC
ACTCCGCTAGGGAGTGCGGCCTGCGCAGCCCCAGGAGGACTGGGTTACC
AAAGCCGTTGAGCCCCCACGGCCCAAGCCTCGTCTAGGATGCAATAGAC
GAGGTGTAAGGACTAGAGGTTAGAGGAGACCCCGTGGAAACAACAACAT
GCGGCCCAAGCCCCCTCGAAGCTGTAGAGGAGGTGGAAGGACTAGAGGT
TAGAGGAGACCCCGCATTTGCATCAAACAGCATATTGACACCTGGGAAT
AGACTGGGAGATCTTCTGCTCTATCTCAACATCAGCTACTAGGCACAGA
GCGCCGAAGTATGTAGCTGGTGGTGAGGAAGAACAC.

In an aspect, a disclosed 3′ UTR can comprise the following sequence or a fragment thereof:

(SEQ ID NO: 13)
ATAACATTGATAGAAAATTTTGTAAATATTTAATGTAATATAGTATAGG
TAAAATTTTTTGAAATTAAGTAAAATTAAGTAGCAAGACTTGATAGTCA
GGCCAGCCGGTTAGGCTGCCACCGAAGGTTGGTAGACGGTGCTGCCTGC
GACCAACCCCAGGAGGACTGGGTTACCAAAGCTGATTCTCCACGGTTGG
AAAGCCTCCCAGAACCGTCTCGGAAGAGGAGTCCCTGCCAACAATGGAG
ATGAAGCCCGTGTCAGATCGCGAAAGCGCCACTTCGCCGAGGAGTGCAA
TCTGTGAGGCCCCAGGAGGACTGGGTAAACAAAGCCGTAAGGCCCCCGC
AGCCCGGGCCGGGAGGAGGTGATGCAAACCCCGGCGAAGGACTAGAGGT
TAGAGGAGACCCTGCGGAAGAAATGAGTGGCCCAAGCTCGCCGAAGCTG
TAAGGCGGGTGGACGGACTAGAGGTTAGAGGAGACCCCACTCTCAAAAG
CATCAAACAACAGCATATTGACACCTGGGAAAAGACTAGGAGATCTTCT
GCTCTATTCCAACATCAGTCACAAGGCACCGAGCGCCGAACACTGTGAC
TGATGGGGGAGAAGACCACAGGATCT.

In an aspect, a disclosed 3′ UTR can comprise the following sequence or a fragment thereof:

(SEQ ID NO: 14)
ACTAGCACAACCAAATAATCAGAAGAAACCCACTACAGGGGGTGATGTG
GCAGCGCACCACGACATCGTGACGGGAAGAGGTCGTCCCCGACGCATCA
TCTCTCTAGGGCATTTTCGTGAGACCCTCATGGCTGCTAAAGGGGCATC
AGCCGTGTAGTAAGAAGGCCCCGACCTACTGCAGCAGCACACACAGTGA
CGGGAAGTTGGTCGCTCCCGACGCAGTTAGGTCAGAGAAAATTTTGTGA
GACCAAAAGGCCTCCTGGAAGGCTCACCAGGGGTTAGGCCATTCTAG.

In an aspect, a disclosed 3′ UTR can comprise the following sequence or a fragment thereof:

(SEQ ID NO: 15)
ATTGAAATGTAAATAGTGTAAATAAATAAAAACAGGTAAGTCAGGCCAA
TCAGTTTTGCCACCGGATGTCAGGTAAACGGTGCTGTCTGTAACCTGGC
CCCAGGCGACTGGGTTATCAAAGCCAACCCGGCTGGGTGCAAAGCCCCT
CATTCCGACTCGGGAGGGTCCCTGGCACGTAGGCTGGAGAGGACGCACA
AGTCAGACCAGAAATGCCACCTGAAAGCATGCTAAAGGTGCTGTCTGTA
CATGCCCCAGGAGGACTGGGTTAACAAAGCTTAACAGCCCCAGCGGCCC
AAACCATGGAGTGCGTGACCATGGCGTAAGGACTAGAGGTTAGAGGAGA
CCCCGCTGTAACTTGGCAAGGCCCAAACCCGCTCAAAGCTGTAGAGACG
GGGGAAGGACTAGAGGTTAGAGGAGACCCCTTGCCGTTAACGCAAACAA
CAGCATATTGACACCTGGAAAGACAGGAGATCCCCTGCTTTTTCAACAC
CAGCCACAAGGCACAGAGCGCCGTAAAGTGTGGCTGGTGGTGAAAAAAT
CACAGGATCT.

In an aspect, a disclosed 3′ UTR can comprise the following sequence or a fragment thereof:

(SEQ ID NO: 16)
ACCCAGACTGTGACAGAGCAAAACCCGGAAGGCTCGTAAAAGATTGTCC
GGAACCAAAAGAAAAGCAAGCAACTCACAGAGATAGAGCTCGGACTGGA
GAGCTCTTTAAACAAAAAAAAAAAAAAAAAAAAAAAAAAAAAAAAAAAA
AAAAAAAAAAAAAGCCAGAATTGAGCTGAACCTGGAGAGCTCATTAAAT
ACAGTCCAGACGAAACAAAACATGACAAAGCAAAGAGGCTGAGCTAAAA
GTTCCCACTACGGGACTGCTTCATAGCGGTTTGTGGGGGGAGGCTAGGA
GGCGAAGCCACAGATCATGGAATGATGCGGCAGCGCGCGAGAGCGACGG
GGAAGTGGTCGCACCCGACGCACCATCCATGAAGCAATACTTCGTGAGA
CCCCCCCTGACCAGCAAAGGGGGCAGACCGGTCAGGGGTGAGGAATGCC
CCCAGAGTGCATTACGGCAGCACGCCAGTGAGAGTGGCGACGGGAAAAT
GGTCGATCCCGACGTAGGGCACTCTGAAAAATTTTGTGAGACCCCCTGC
ATCATGATAAGGCCGAACATGGTGCATGAAAGGGGAGGCCCCCGGAAGC
ACGCTTCCGGGAGGAGGGAAGAGAGAAATTGGCAGCTCTCTTCAGGATT
TTTCCTCCTCCTATACAAAATTCCCCCTCGGTAGAGGGGGGGCGGTTCT
TGTTCTCCCTGAGCCACCATCACCCAGACACAGGTAGTCTGACAAGGAG
GTGATGTGTGACTCGGAAAAACACCCGCT.

In an aspect, a disclosed 3′ UTR can comprise the following sequence or a fragment thereof:

(SEQ ID NO: 17)
AAGATAGTATTATAGTTAGTTTAGTGTAAATAGGATTTATTGAGAATGG
AAGTCAGGCCAGATTAATGCTGCCACCGGAAGTTGAGTAGACGGTGCTG
CCTGCGGCTCAACCCCAGGAGGACTGGGTGACCAAAGCTGCGAGGTGAT
CCACGTAAGCCCTCAGAACCGTCTCGGAAGGAGGACCCCACGTGCTTTA
GCCTCAAAGCCCAGTGTCAGACCACACTTTAATGTGCCACTCTGCGGAG
AGTGCAGTCTGCGATAGTGCCCCAGGTGGACTGGGTTAACAAAGGCAAA
ACATCGCCCCACGCGGCCATAACCCTGGCTATGGTGTTAACCAGGGAGA
AGGGACTAGAGGTTAGAGGAGACCCCGCGTAAAAAAGTGCACGGCCCAA
CTTGGCTGAAGCTGTAAGCCAAGGGAAGGACTAGAGGTTAGAGGAGACC
CCGTGCCAAAAACACCAAAAGAAACAGCATATTGACACCTGGGATAGAC
TAGGGGATCTTCTGCTCTGCACAACCAGCCACACGGCACAGTGCGCCGA
CATAGGTGGCTGGTGGTGCTAGAACACAGGATCT.

In an aspect, a disclosed 3′ UTR can comprise the following sequence or a fragment thereof:

(SEQ ID NO: 18)
GCACCAATCTTAGTGTTGTCAGGCCTGCTAGTCAGCCACAGCTTGGGGA
AAGCTGTGCAGCCTGTGACCCCCCCAGGAGAAGCTGGGAAACCAAGCCT
ATAGTCAGGCCGAGAACGCCATGGCACGGAAGAAGCCATGCTGCCTGTG
AGCCCCTCAGAGGACACTGAGTCAAAAAACCCCACGCGCTTGGAGGCGC
AGGATGGGAAAAGAAGGTGGCGACCTTCCCCACCCTTCAATCTGGGGCC
TGAACTGGAGATCAGCTGTGGATCTCCAGAAGAGGGACTAGTGGTTAGA
GGAGACCCCCCGGAAAACGCAAAACAGCATATTGACGCTGGGAAAGACC
AGAGACTCCATGAGTTTCCACCACGCTGGCCGCCAGGCACAGATCGCCG
AATAGCGGCGGCCGGTGTGGGGAAATCCATGGGTCT.

In an aspect, a disclosed 3′ UTR can comprise the following sequence or a fragment thereof:

(SEQ ID NO: 19)
AACACCATCTAACAGGAATAACCGGGATACAAACCACGGGTGGAGAACC
GGACTCCCCACAACCTGAAACCGGGATATAAACCACGGCTGGAGAACCG
GACTCCGCACTTAAAATGAAACAGAAACCGGGATAAAAACTACGGATGG
AGAACCGGACTCCACACATTGAGACAGAAGAAGTTGTCAGCCCAGAACC
CCACACGAGTTTTGCCACTGCTAAGCTGTGAGGCAGTGCAGGCTGGGAC
AGCCGACCTCCAGGTTGCGAAAAACCTGGTTTCTGGGACCTCCCACCCC
AGAGTAAAAAGAACGGAGCCTCCGCTACCACCCTCCCACGTGGTGGTAG
AAAGACGGGGTCTAGAGGTTAGAGGAGACCCTCCAGGGAACAAATAGTG
GGACCATATTGACGCCAGGGAAAGACCGGAGTGGTTCTCTGCTTTTCCT
CCAGAGGTCTGTGAGCACAGTTTGCTCAAGAATAAGCAGACCTTTGGAT
GACAAACACAAAACCACT.

In an aspect, the one or more disclosed Flavivirus genetic elements can comprise subgenomic Flavivirus RNA (sfRNA) elements. In an aspect, the one or more disclosed Flavivirus genetic elements can comprise recombinant or mutant subgenomic Flavivirus RNA (sfRNA) elements.

In an aspect, the one or more disclosed sfRNA elements can comprise a Flavivirus XRN1-resistant RNA (xrRNA) element, a Flavivirus sfRNA dumbbell (DB) RNA element, a Flavivirus 3′ stem loop (3′ SL) element, or any combination thereof.

In an aspect, a disclosed Flavivirus 3′ stem loop element can comprise a recombinant, mutant, or wild-type 3′ SL element. In an aspect, a disclosed Flavivirus 3′ stem loop element can comprise a 3′ SL element from a recombinant, mutant, or wild-type Flavivirus. In an aspect, a disclosed Flavivirus 3′ stem loop element can comprise the sequence set forth in SEQ ID NO:01 or a fragment thereof. For example, in an aspect, a disclosed Flavivirus 3′ stem loop element can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:01, or a sequence having at least 40%-60%, at least 60%-80%, at least 80%-90%, or at least 90%-100% identity to the sequence set forth in SEQ ID NO:01. In an aspect, a disclosed 3′ SL element can comprise a 3′ SL of Dengue Virus 2.

In an aspect, a disclosed 3′ SL element can comprise the following sequence or a fragment thereof:

(SEQ ID NO: 01)
CAGCAUAUUGACGCUGGGAAAGACCAGAGAUCCUGCUGUCUCCUCAGCA
UCAUUCCAGGCACAGAACGCCAGAAAAUGGAAUGGUGCUGUUGAAUCAA
CAGGUUCU.

In an aspect, a disclosed Flavivirus 3′ SL element can comprise one or more mutations. In an aspect, a disclosed mutant 3′ SL element can comprise a mutant 3′ SL element of Dengue Virus 2. In an aspect, a disclosed mutant xrRNA element can comprise the sequence set forth in SEQ ID NO:39 or a fragment thereof. In an aspect, a disclosed 3′ SL element can comprise a sequence having at least 80%, 85%, 86%, 87%, 88%, 89%, 90%, 91%, 92%, 93%, 94%, 95%, 96%, 97%, 98%, 99% identity to the sequence set forth in SEQ ID NO:39 or a fragment thereof. In an aspect, a disclosed mutant 3′ SL can comprise the following sequence or a fragment thereof:

(SEQ ID NO: 39)
GCTAGCAGGCAAAACTAACATGAAACAAGGCTAAAAGTCAGGTCGGATC
AAGCCATAGTACGGAAAAAACTATGCTACCTGTGAGCCCCGTCCAAGGA
CGTTAAAAGAAGTCAGGCCATCACAAATGCCACAGCTTGAGTAAACTGT
GCAGCCTGTAGCTCCACCTGAGAAGGTGTAAAAAATCTGGGAGGCCACA
AACCATGGAAGCTGTACGCATGGCGTAGTGGACTAGCGGTTAGAGGAGA
CCCCTCCCTTACAAATCGCAGCAACAACGGGGGCCCAAGGTGAGATGAA
GCTGTAGTCTCACTGGAAGGACTAGAGGTTAGAGGAGACCCCCCCAAAA
CAAAAAACAGCATATTGACG.

In an aspect, a disclosed Flavivirus XRN1-resistant RNA (xrRNA) element can comprise a recombinant, mutant, or wild-type xrRNA element. In an aspect, a disclosed Flavivirus XRN1-resistant RNA (xrRNA) element can comprise a XRN1-resistant RNA (xrRNA) element from a recombinant, mutant, or wild-type Flavivirus. In an aspect, a disclosed xrRNA element can comprise a xrRNA element of Dengue Virus 2. In an aspect, a disclosed Flavivirus XRN1-resistant RNA (xrRNA) element can comprise the sequence set forth in SEQ ID NO:02 or a fragment thereof or SEQ ID NO:03 or fragment thereof. In an aspect, a disclosed Flavivirus XRN1-resistant RNA (xrRNA) element can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:02 or SEQ ID NO:03. In an aspect, a disclosed Flavivirus XRN1-resistant RNA (xrRNA) element can comprise a sequence having at least 40%-60%, at least 60%-80%, at least 80%-90%, or at least 90%-100% identity to the sequence set forth in SEQ ID NO:02 or SEQ ID NO:03.

In an aspect, a disclosed xrRNA element can comprise the following sequence or a fragment thereof:

(SEQ ID NO: 02)
UAAAAGUCAGGUCGGAUCAAGCCAUAGUACGGAAAAAACUAUGCUACCU
GUGAGCCCCGUCCAAGGACGU.

In an aspect, a disclosed xrRNA element can comprise the following sequence or a fragment thereof:

(SEQ ID NO: 03)
GUCAGGCCAUCACAAAUGCCACAGCUUGAGUAAACUGUGCAGCCUGUAG
CUCCACCUGAGAAGGUGU.

In an aspect, a disclosed Flavivirus xrRNA element can comprise one or more mutations. In an aspect, a disclosed mutant xrRNA element can comprise a mutant xrRNA element of Dengue Virus 2. In an aspect, a disclosed mutant xrRNA element can comprise the sequence set forth in SEQ ID NO:37 or a fragment thereof. In an aspect, a disclosed mutant xRNA element can comprise a sequence having at least 80%, 85%, 86%, 87%, 88%, 89%, 90%, 91%, 92%, 93%, 94%, 95%, 96%, 97%, 98%, 99% identity to the sequence set forth in SEQ ID NO:37 or a fragment thereof. In an aspect, a disclosed mutant xrRNA can comprise the following sequence or a fragment thereof:

(SEQ ID NO: 37)
GCTAGCAGGCAAAACTAACATGAAACAAGGCAAAAAATCTGGGAGGCCA
CAAACCATGGAAGCTGTACGCATGGCGTAGTGGACTAGCGGTTAGAGGA
GACCCCTCCCTTACAAATCGCAGCAACAACGGGGGCCCAAGGTGAGATG
AAGCTGTAGTCTCACTGGAAGGACTAGAGGTTAGAGGAGACCCCCCCAA
AACAAAAAACAGCATATTGACGCTGGGAAAGACCAGAGATCCTGCTGTC
TCCTCAGCATCATTCCAGGCACAGAACGCCAGAAAATGGAATGGTGCTG
TTGAATCAACAGGTTCT.

In an aspect, a disclosed sfRNA dumbbell (DB) RNA element can comprise a recombinant, mutant, or wild-type DB RNA element. In an aspect, a disclosed sfRNA dumbbell (DB) RNA element can comprise a DB RNA element from a recombinant, mutant, or wild-type Flavivirus. In an aspect, a disclosed Flavivirus sfRNA dumbbell (DB) RNA element can comprise the sequence set forth in SEQ ID NO:04 or SEQ ID NO:05. In an aspect, a disclosed Flavivirus sfRNA dumbbell (DB) RNA element can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:04 or a fragment thereof or SEQ ID NO:05 or a fragment thereof. In an aspect, a disclosed Flavivirus sfRNA dumbbell (DB) RNA element can comprise a sequence having at least 40%-60%, at least 60%-80%, at least 80%-90%, or at least 90%-100% identity to the sequence set forth in SEQ ID NO:04 or a fragment thereof or SEQ ID NO:05 or a fragment thereof.

In an aspect, a disclosed DB element can comprise a DB element of Dengue Virus 2. In an aspect, a disclosed DB element can comprise the following sequence or a fragment thereof:

(SEQ ID NO: 04)
GGAGGCCACAAACCAUGGAAGCUGUACGCAUGGCGUAGUGGACUAGCGG
UUAGAGGAGACCCCUCCC.

In an aspect, a disclosed DB element can comprise the following sequence or a fragment thereof:

(SEQ ID NO: 05)
GGGGGCCCAAGGUGAGAUGAAGCUGUAGUCUCACUGGAAGGACUAGAGG
UUAGAGGAGACCCCCCC.

In an aspect, a disclosed Flavivirus sfRNA dumbbell (DB) RNA element can comprise one or more mutations. In an aspect, disclosed mutant Flavivirus sfRNA dumbbell (DB) RNA element can comprise the sequence set forth in SEQ ID NO:06 or a fragment thereof or SEQ ID NO:07 or a fragment thereof. In an aspect, a disclosed mutant Flavivirus sfRNA dumbbell (DB) RNA element can comprise a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in SEQ ID NO:06 or a fragment or SEQ ID NO:07 or a fragment thereof. In an aspect, a disclosed mutant Flavivirus sfRNA dumbbell (DB) RNA element can comprise a sequence having at least 40%-60%, at least 60%-80%, at least 80%-90%, or at least 90%-100% identity to the sequence set forth in SEQ ID NO:06 or a fragment thereof or SEQ ID NO:07 or a fragment thereof.

In an aspect, a disclosed DB RNA element can comprise the following sequence or a fragment thereof:

(SEQ ID NO: 06)
GGAGGCCACAAACCAUGGAAGCUGUACGCAUGGCGUAGUGAACUAGCAG
UUAGAGGAGACCCCUCCC.

In an aspect, a disclosed DB RNA element can comprise the following sequence or a fragment thereof:

(SEQ ID NO: 07)
GGAGGCCACAAACCAUGGAAGCUGUACGCAUGGCGUAGUGGACUAGCGG
UUAGACGACACCCCUCCC.

In an aspect, a disclosed mutant DB element can comprise a mutant DB element of Dengue Virus 2. In an aspect, a disclosed mutant DB element can comprise the sequence set forth in SEQ ID NO:38 or a fragment thereof. In an aspect, a disclosed mutant DB element can comprise a sequence having at least 80%, 85%, 86%, 87%, 88%, 89%, 90%, 91%, 92%, 93%, 94%, 95%, 96%, 97%, 98%, 99% identity to the sequence set forth in SEQ ID NO:38. In an aspect, a disclosed mutant xrRNA can comprise the following sequence or a fragment thereof:

(SEQ ID NO: 38)
GCTAGCAGGCAAAACTAACATGAAACAAGGCTAAAAGTCAGGTCGGATC
AAGCCATAGTACGGAAAAAACTATGCTACCTGTGAGCCCCGTCCAAGGA
CGTTAAAAGAAGTCAGGCCATCACAAATGCCACAGCTTGAGTAAACTGT
GCAGCCTGTAGCTCCACCTGAGAAGGTGTAAAAAATCTGAAAACAAAAA
ACAGCATATTGACGCTGGGAAAGACCAGAGATCCTGCTGTCTCCTCAGC
ATCATTCCAGGCACAGAACGCCAGAAAATGGAATGGTGCTGTTGAATCA
ACAGGTTCT.

In an aspect, a disclosed nucleic acid molecule can comprise a nucleic acid sequence encoding a transgene. In an aspect, a disclosed transgene can encode a polypeptide or an RNA.

In an aspect, a disclosed encoded RNA can comprise ribosomal RNA (rRNA), transfer RNA (tRNA), heterogeneous nuclear RNA (hnRNA), small nuclear RNA (snRNA), small nucleolar RNA (snoRNA), micro RNA (miRNA), Piwi-interacting RNA (piRNA), small interfering RNA (siRNA), short hairpin RNA (shRNA), singe guide RNA (sgRNA), non-coding RNA (ncRNA), long non-coding RNA (lncRNA), 7SL, Xist, short enhancer RNA (eRNA), circular RNA, intergenic RNA, or any combination thereof. In an aspect, a disclosed encoded RNA can comprise lncRNA, siRNA, shRNA, sgRNA, circular RNA, snoRNA, miRNA, or any combination thereof. In an aspect, a disclosed encoded RNA can comprise a functional non-coding RNA element.

In an aspect, a disclosed encoded transgene can comprise any gene with a gene product that is directly or indirectly linked to one or more genetic diseases. Such genes include but are not limited to the following: dystrophin including mini- and micro-dystrophins (DMD); titin (TTN); titin cap (TCAP) α-sarcoglycan (SGCA), β-sarcoglycan (SGCB), γ-sarcoglycan (SGCG) or 6-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI 3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); D-N-acetylhexosaminidase B (HEXB); 0-glucocerebrosidase (GBA); adenosine monophosphate deaminase 1 (AMPD1); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine(N-acetyl)-6-sulfatase (GNS); galactosamine(N-acetyl)-6-sulfatase (GALNS); 0-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase a class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel a subunit 4 (SCN4A); sodium voltage-gated channel β subunit 4 (SCN4B); sodium voltage-gated channel α subunit 5 (SCN5A); sodium voltage-gated channel α subunit 4 (SCN4A); calcium voltage-gated channel subunit α1c (CACNAlC); calcium voltage-gated channel subunit α1s (CACNAlS); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); camitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGEl); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase R (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic α4 subunit (CHRNA4); cholinergic receptor nicotinic α5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic 31 subunit (CHRNB1); cholinergic receptor nicotinic 32 subunit (CHRNB2); cholinergic receptor nicotinic 03 subunit (CHRNB3); cholinergic receptor nicotinic 04 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCHI); notch 3 (NOTCH3); dystrobrevin a (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH:ubiquinone oxidoreductase core subunit 1 (MT-ND1); mitochondrially encoded NADH:ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGF03); transforming growth factor β receptor, type I (TGFβR1); transforming growth factor β receptor, type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

Disclosed herein is an isolated nucleic acid molecule, comprising one or more subgenomic Flavivirus RNA (sfRNA) elements. In an aspect, the one or more disclosed subgenomic sfRNA elements can comprise XRN1-resistant RNA (xrRNA) elements, sfRNA dumbbell (DB) RNA elements, 3′ stem loop (3′ SL) elements, or any combination thereof.

Disclosed herein is an isolated nucleic acid molecule, comprising one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is an isolated nucleic acid molecule, comprising a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is an isolated nucleic acid molecule, comprising one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is an isolated nucleic acid molecule, comprising one or more Flavivirus 3′ untranslated region (3′ UTR) elements.

Disclosed herein is an isolated nucleic acid molecule, comprising one or more Flavivirus XRN1-resistant RNA (xrRNA) elements. In an aspect, a disclosed isolated nucleic acid molecule can further comprise one or more Flavivirus sfRNA dumbbell (DB) RNA elements, one or more Flavivirus 3′ stem loop (3′ SL) elements, or any combination thereof.

Disclosed herein is an isolated nucleic acid molecule, comprising one or more Flavivirus sfRNA dumbbell (DB) RNA elements. In an aspect, a disclosed isolated nucleic acid molecule can further comprise one or more Flavivirus XRN1-resistant RNA (xrRNA) elements, one or more Flavivirus 3′ stem loop (3′ SL) elements, or any combination thereof.

Disclosed herein is an isolated nucleic acid molecule, comprising one or more Flavivirus 3′ stem loop (3′ SL) elements. In an aspect, a disclosed isolated nucleic acid molecule further comprising one or more Flavivirus sfRNA dumbbell (DB) RNA elements, one or more Flavivirus XRN1-resistant RNA (xrRNA) elements, or any combination thereof.

Disclosed herein is an isolated nucleic acid molecule, comprising one or more 3′ UTRs of a Flavivirus, wherein the one or more 3′ UTRs comprise one or more subgenomic Flavivirus RNA (sfRNA) elements. In an aspect, a disclosed isolated nucleic acid molecule further comprising further comprising one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is an isolated nucleic acid molecule, comprising one or more 3′ UTRs of a Flavivirus, wherein the one or more 3′ UTRs comprises one or more xrRNA elements, one or more DB RNA elements, a 3′ stem loop, or any combination thereof.

Disclosed herein is an isolated nucleic acid molecule, comprising at least two Flavivirus XRN1-resistant RNA (xrRNA) element, at least two Flavivirus sfRNA dumbbell (DB) RNA elements, and a Flavivirus 3′ stem loop (3′ SL) element. In an aspect, a disclosed xrRNA element can comprise the sequence set forth in SEQ ID NO:01 and SEQ ID NO:02. In an aspect, a disclosed DB RNA element can comprise the sequence set forth in SEQ ID NO:04 or fragment thereof or SEQ ID NO:05 or fragment thereof. In an aspect, a disclosed 3′ SL element can comprise the sequence set forth in SEQ ID NO:01 or a fragment thereof.

Disclosed herein is an isolated nucleic acid molecule, comprising a nucleic acid sequence encoding a polypeptide or an RNA molecule, and a nucleic acid sequence encoding one or more Flavivirus genetic elements.

Disclosed herein is an isolated nucleic acid molecule, comprising a nucleic acid sequence encoding a polypeptide or an RNA molecule, and a nucleic acid sequence encoding one or more subgenomic Flavivirus RNA (sfRNA) elements.

Disclosed herein is an isolated nucleic acid molecule, comprising a nucleic acid sequence encoding a polypeptide or an RNA molecule, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is an isolated nucleic acid molecule, comprising a nucleic acid sequence encoding a polypeptide or an RNA molecule, and a nucleic acid sequence encoding a Flavivirus 3′ untranslated region (3′ UTR) element.

Disclosed herein is an isolated nucleic acid molecule, comprising a nucleic acid sequence encoding a polypeptide or an RNA molecule, and a nucleic acid sequence encoding a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is an isolated nucleic acid molecule, comprising a nucleic acid sequence encoding a polypeptide or an RNA molecule, and a nucleic acid sequence encoding one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is an isolated nucleic acid molecule, comprising a nucleic acid sequence encoding a polypeptide or an RNA molecule, and a nucleic acid sequence encoding one or more Flavivirus 3′ untranslated region (3′ UTR) elements.

Disclosed herein is an isolated nucleic acid molecule, comprising a nucleic acid sequence encoding a polypeptide or an RNA molecule, and a nucleic acid sequence encoding one or more Flavivirus XRN1-resistant RNA (xrRNA) elements. In an aspect, a disclosed isolated nucleic acid molecule can further comprise one or more Flavivirus sfRNA dumbbell (DB) RNA elements, one or more Flavivirus 3′ stem loop (3′ SL) elements, or any combination thereof

Disclosed herein is an isolated nucleic acid molecule, comprising a nucleic acid sequence encoding a polypeptide or an RNA molecule, and a nucleic acid sequence encoding one or more Flavivirus sfRNA dumbbell (DB) RNA elements. In an aspect, a disclosed isolated nucleic acid molecule can further comprise one or more Flavivirus XRN1-resistant RNA (xrRNA) elements, one or more Flavivirus 3′ stem loop (3′ SL) elements, or any combination thereof.

Disclosed herein is an isolated nucleic acid molecule, comprising a nucleic acid sequence encoding a polypeptide or an RNA molecule, and a nucleic acid sequence encoding one or more Flavivirus 3′ stem loop (3′ SL) elements. In an aspect, a disclosed isolated nucleic acid molecule can further comprise one or more Flavivirus sfRNA dumbbell (DB) RNA elements, one or more Flavivirus XRN1-resistant RNA (xrRNA) elements, or any combination thereof.

Disclosed herein is an isolated nucleic acid molecule, comprising a nucleic acid sequence encoding a polypeptide or an RNA molecule, and a nucleic acid sequence encoding one or more 3′ UTRs of a Flavivirus, wherein the one or more 3′ UTRs comprise one or more subgenomic Flavivirus RNA (sfRNA) elements. In an aspect, a disclosed isolated nucleic molecule acid can further comprise one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is a nucleic acid molecule, comprising a nucleic acid sequence encoding a polypeptide or an RNA molecule, and a nucleic acid sequence encoding one or more 3′ UTRs of a Flavivirus, wherein the one or more 3′ UTRs comprises one or more xrRNA elements, one or more DB RNA elements, a 3′ stem loop, or any combination thereof.

Disclosed herein is an isolated nucleic acid molecule, comprising a nucleic acid sequence encoding a polypeptide or an RNA molecule, and a nucleic acid sequence encoding at least two Flavivirus XRN1-resistant RNA (xrRNA) element, at least two Flavivirus sfRNA dumbbell (DB) RNA elements, and a Flavivirus 3′ stem loop (3′ SL) element.

In an aspect, an isolated nucleic acid molecule can further comprise a nucleic acid sequence encoding a promoter operably linked to the polypeptide or the RNA molecule.

In an aspect of a disclosed isolated nucleic acid molecule, a disclosed Flavivirus sequence can be positioned 3′ of ORF for the transgene, 5′ of ORF for the transgene, or 3′ of a promoter the transgene. In an aspect of a disclosed isolated nucleic acid molecule, a disclosed Flavivirus sequence can be positioned anywhere.

Disclosed herein is an isolated nucleic acid molecule, comprising: one or more Flavivirus genetic elements, wherein the one or more Flavivirus genetic elements comprise two XRN1-resistant RNA (xrRNA) elements, two dumbbell (DB) RNA element, and a 3′ stem loop (3′ SL) element, wherein the xrRNA elements comprise the sequence of SEQ ID NO:02 and SEQ ID NO:03, the two DB elements comprise the sequence of SEQ ID NO:04 and SEQ ID NO:05, and the 3′ SL element comprises the sequence of SEQ ID NO:01.

Disclosed herein is an isolated nucleic acid molecule comprising the sequence of SEQ ID NO:02, SEQ ID NO:03, SEQ ID NO:04, SEQ ID NO:05, and sequence of SEQ ID NO:01.

Disclosed herein is an isolated nucleic acid molecule comprising a nucleic acid sequence comprising one or more of the sequences set forth in SEQ ID NO:01-SEQ ID NO:05 and a nucleic acid sequence encoding a transgene. Disclosed herein is an isolated nucleic acid molecule comprising a nucleic acid sequence comprising the sequence set forth in each of SEQ ID NO:01-SEQ ID NO:05 and a nucleic acid sequence encoding a transgene.

Disclosed herein is an isolated nucleic acid molecule, comprising a nucleic acid sequence encoding a transgene, and a nucleic acid sequence encoding one or more Flavivirus genetic elements.

Disclosed herein is an isolated nucleic acid molecule, comprising a nucleic acid sequence encoding a transgene, and a nucleic acid sequence encoding one or more subgenomic Flavivirus RNA (sfRNA) elements.

Disclosed herein is an isolated nucleic acid molecule, comprising a nucleic acid sequence encoding transgene, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is an isolated nucleic acid molecule, comprising a nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is an expression cassette, comprising a nucleic acid sequence encoding a polypeptide or an RNA molecule operably linked to a promoter, and a nucleic acid sequence encoding one or more Flavivirus genetic elements.

Disclosed herein is an expression cassette, comprising a nucleic acid sequence encoding a polypeptide or an RNA molecule operably linked to a promoter, and a nucleic acid sequence encoding one or more subgenomic Flavivirus RNA (sfRNA) elements.

Disclosed herein is an expression cassette, comprising a nucleic acid sequence encoding a polypeptide or an RNA molecule operably linked to a promoter, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is an expression cassette, comprising a nucleic acid sequence encoding a polypeptide or an RNA molecule operably linked to a promoter, and a nucleic acid sequence encoding a Flavivirus 3′ untranslated region (3′ UTR) element.

Disclosed herein is an expression cassette, comprising a nucleic acid sequence encoding a polypeptide or an RNA molecule operably linked to a promoter, and a nucleic acid sequence encoding a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein an expression cassette, comprising a nucleic acid sequence encoding a polypeptide or an RNA molecule operably linked to a promoter, and a nucleic acid sequence encoding one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is an expression cassette, comprising a nucleic acid sequence encoding a polypeptide or an RNA molecule operably linked to a promoter, and a nucleic acid sequence encoding one or more Flavivirus 3′ untranslated region (3′ UTR) elements.

Disclosed herein is an expression cassette, comprising a nucleic acid sequence encoding a polypeptide or an RNA molecule operably linked to a promoter, and a nucleic acid sequence encoding one or more Flavivirus XRN1-resistant RNA (xrRNA) elements. In an aspect of a disclosed expression cassette, a disclosed nucleic acid sequence can further comprise one or more Flavivirus sfRNA dumbbell (DB) RNA elements, one or more Flavivirus 3′ stem loop (3′ SL) elements, or any combination thereof.

Disclosed herein is an expression cassette, comprising a nucleic acid sequence encoding a polypeptide or an RNA molecule operably linked to a promoter, and a nucleic acid sequence encoding one or more Flavivirus sfRNA dumbbell (DB) RNA elements. In an aspect of a disclosed expression cassette, a disclosed nucleic acid sequence can further comprise one or more Flavivirus XRN1-resistant RNA (xrRNA) elements, one or more Flavivirus 3′ stem loop (3′ SL) elements, or any combination thereof.

Disclosed herein is an expression cassette, comprising a nucleic acid sequence encoding a polypeptide or an RNA molecule operably linked to a promoter, and a nucleic acid sequence encoding one or more Flavivirus 3′ stem loop (3′ SL) elements. In an aspect of a disclosed expression cassette, a disclosed nucleic acid sequence can further comprise one or more Flavivirus sfRNA dumbbell (DB) RNA elements, one or more Flavivirus XRN1-resistant RNA (xrRNA) elements, or any combination thereof.

Disclosed herein is an expression cassette, comprising a nucleic acid sequence encoding a polypeptide or an RNA molecule operably linked to a promoter, and a nucleic acid sequence encoding one or more 3′ UTRs of a Flavivirus, wherein the one or more 3′ UTRs comprise one or more subgenomic Flavivirus RNA (sfRNA) elements. In an aspect of a disclosed expression cassette, a disclosed nucleic acid sequence can further comprise one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is an expression cassette, comprising a nucleic acid sequence encoding a polypeptide or an RNA molecule operably linked to a promoter, and a nucleic acid sequence encoding one or more 3′ UTRs of a Flavivirus, wherein the one or more 3′ UTRs comprises one or more xrRNA elements, one or more DB RNA elements, a 3′ stem loop, or any combination thereof.

Disclosed herein is an expression cassette, comprising a nucleic acid sequence encoding a polypeptide or an RNA molecule operably linked to a promoter, and a nucleic acid sequence encoding at least two Flavivirus XRN1-resistant RNA (xrRNA) element, at least two Flavivirus sfRNA dumbbell (DB) RNA elements, and a Flavivirus 3′ stem loop (3′ SL) element.

Disclosed herein is an expression cassette, comprising a nucleic acid molecule comprising one or more Flavivirus genetic elements, wherein the one or more Flavivirus genetic elements comprise two XRN1-resistant RNA (xrRNA) elements, two dumbbell (DB) RNA element, and a 3′ stem loop (3′ SL) element, wherein the xrRNA elements comprise the sequence of SEQ ID NO:02 and SEQ ID NO:03, the two DB elements comprise the sequence of SEQ ID NO:04 and SEQ ID NO:05, and the 3′ SL element comprises the sequence of SEQ ID NO:01.

Disclosed herein is an expression cassette, comprising a nucleic acid molecule having the sequence of SEQ ID NO:02, SEQ ID NO:03, SEQ ID NO:04, SEQ ID NO:05, and sequence of SEQ ID NO:01.

In an aspect, a disclosed nucleic acid sequence is CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a disclosed isolated nucleic acid molecule can comprise a single stranded DNA molecule, or a single stranded RNA template, or a double stranded DNA template.

In an aspect, a disclosed isolated nucleic acid molecule can comprise a polyA tail.

In an aspect, a disclosed isolated nucleic acid molecule can restore one or more aspects of cellular homeostasis and/or cellular functionality and/or metabolic dysregulation. In an aspect, a disclosed isolated nucleic acid molecule can restore the functionality and/or structural integrity of a missing, deficient, and/or mutant protein or enzyme.

In an aspect, a disclosed isolated nucleic acid molecule can increase the stability or half-life of an RNA molecule. In an aspect, a disclosed isolated nucleic acid molecule can increase the translation efficiency of an RNA molecule. In an aspect, a disclosed isolated nucleic acid molecule can enhance gene therapy.

In an aspect, upon transcription, a disclosed isolated nucleic acid molecule can be present within an RNA molecule.

In an aspect, a disclosed Flavivirus can comprise any Flavivirus. In an aspect, a disclosed Flavivirus can comprise a recombinant Flavivirus or a mutant Flavivirus. In an aspect, a disclosed Flavivirus can comprise Dengue virus type 1 (DENV1), Dengue virus type 2 (DENV2), Dengue virus type 3 (DENV3), Dengue virus type 4 (DENV4), Japanese encephalitis virus (JEV), Murray Valley encephalitis virus (MVEV), Powassan virus (POWV), St. Louis encephalitis virus (SLEV), Tick-borne Encephalitis (TBEV), West Nile virus (WNV), Zika virus (ZIKV), and Yellow fever virus (YFV). The table below provides the sequence identifier for the enumerated Flavivirus.

| Sequence Identifier | Flavivirus (GenBank Accession No.) |
|---|---|
| SEQ ID NO: 20 | Dengue Virus 1 (West Pac) (U88535.1) |
| SEQ ID NO: 21 | Dengue Virus 2 (strain 16681) (KU725663.1) |
| SEQ ID NO: 22 | Dengue Virus 3 (UNC3001) (JQ411814.1) |
| SEQ ID NO: 23 | Dengue Virus 4 (rDENV4) (KJ160504.1) |
| SEQ ID NO: 24 | Japanese encephalitis virus (JEV) |
| | strain SA-14 (M55506.1) |
| SEQ ID NO: 25 | Murray Valley encephalitis virus |
| | (MVEV) (NC000943.1) |
| SEQ ID NO: 26 | Powassan virus (POWV) strain |
| | P0375 (KU886216.1) |
| SEQ ID NO: 27 | Saint Louis encephalitis virus |
| | (SLEV) (NC007580.2) |
| SEQ ID NO: 28 | Tick-borne encephalitis virus |
| | (TBEV) strain Neudoerfl (U27495.1) |
| SEQ ID NO: 29 | West Nile virus (WNV) (M12294.2) |
| SEQ ID NO: 30 | Zika virus (ZIKV) strain |
| | H/PF/2013 (KJ776791.) |
| SEQ ID NO: 31 | Yellow fever virus (YFV) |
| | strain 17D (MT107250.1) |

In an aspect, a disclosed Flavivirus can comprise a recombinant or mutant Dengue virus type 1 (DENV1), a recombinant or mutant Dengue virus type 2 (DENV2), a recombinant or mutant Dengue virus type 3 (DENV3), a recombinant or mutant Dengue virus type 4 (DENV4), a recombinant or mutant Japanese encephalitis virus (JEV), a recombinant or mutant Murray Valley encephalitis virus (MVEV), a recombinant or mutant Powassan virus (POWV), a recombinant or mutant St. Louis encephalitis virus (SLEV), a recombinant or mutant Tick-borne Encephalitis (TBEV), a recombinant or mutant West Nile virus (WNV), a recombinant or mutant Zika virus (ZIKV), and a recombinant or mutant Yellow fever virus (YFV).

In an aspect, a disclosed Flavivirus can comprise Apoi virus, Aroa virus, Bamaga virus, Banzi virus, Bouboui virus, Bukalasa bat virus, Cacipacore virus, Carey Island virus, Cowbone Ridge virus, Dakar bar virus, Dengue virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Saint Louis encephalitis virus, Sal Viega virus, San Perlita virus, Saumarez Reef virus, Sepik virus, Tick-borne encephalitis virus, Tyulenly virus, Uganda S virus, Usuta virus, Wesselsbron virus, West Nile virus, Yaounde virus, Yellow fever virus, Yokose virus, Zika virus, or any combination thereof

In an aspect, a disclosed Flavivirus can comprise a recombinant or mutant Apoi virus, a recombinant or mutant Aroa virus, a recombinant or mutant Bamaga virus, a recombinant or mutant Banzi virus, a recombinant or mutant Bouboui virus, a recombinant or mutant Bukalasa bat virus, a recombinant or mutant Cacipacore virus, a recombinant or mutant Carey Island virus, a recombinant or mutant Cowbone Ridge virus, a recombinant or mutant Dakar bar virus, a recombinant or mutant Dengue virus, a recombinant or mutant Edge Hill virus, a recombinant or mutant Entebbe bat virus, a recombinant or mutant Gadgets Gully virus, a recombinant or mutant Ilheus virus, a recombinant or mutant Israel turkey meningoencephalomyelitis virus, a recombinant or mutant Japanese encephalitis virus, a recombinant or mutant Jugra virus, a recombinant or mutant Jutiapa virus, a recombinant or mutant Kadam virus, a recombinant or mutant Kedougou virus, a recombinant or mutant Kokobera virus, a recombinant or mutant Koutango virus, a recombinant or mutant Kyasanur Forest disease virus, a recombinant or mutant Langat virus, a recombinant or mutant Louping ill virus, a recombinant or mutant Meaban virus, a recombinant or mutant Modoc virus, a recombinant or mutant Montana myotis leukoencephalitis virus, a recombinant or mutant Murray Valley encephalitis virus, a recombinant or mutant Ntaya virus, a recombinant or mutant Omsk hemorrhagic fever virus, a recombinant or mutant Phnom Penh bat virus, a recombinant or mutant Powassan virus, a recombinant or mutant Rio Bravo virus, a recombinant or mutant Royal Farm virus, a recombinant or mutant Saboya virus, a recombinant Saint Louis encephalitis virus, a recombinant or mutant Sal Viega virus, a recombinant or mutant San Perlita virus, a recombinant or mutant Saumarez Reef virus, a recombinant or mutant Sepik virus, a recombinant or mutant Tick-borne encephalitis virus, a recombinant or mutant Tyulenly virus, a recombinant or mutant Uganda S virus, a recombinant or mutant Usuta virus, a recombinant or mutant Wesselsbron virus, a recombinant or mutant West Nile virus, a recombinant or mutant Yaounde virus, a recombinant or mutant Yellow fever virus, a recombinant or mutant Yokose virus, a recombinant or mutant Zika virus, or any combination thereof.

In an aspect, a disclosed Flavivirus element can comprise a partial sequence and a complete sequence for the element.

In an aspect, a disclosed Flavivirus genetic element can comprise a genetic element of any Flavivirus. In an aspect, a disclosed Flavivirus genetic element can comprise a genetic element (e.g., 3′ UTR, 3′ SL, xrRNA, and/or DB) of a recombinant or a mutant Flavivirus. In an aspect, a disclosed Flavivirus RNA genetic element can comprise a recombinant or mutant genetic element. In an aspect, a disclosed subgenomic Flavivirus RNA (sfRNA) element can comprise a sfRNA of any Flavivirus. In an aspect, a disclosed subgenomic Flavivirus RNA (sfRNA) element (e.g., 3′ SL, xrRNA, and/or DB) can comprise a sfRNA of any recombinant Flavivirus. In an aspect, a disclosed subgenomic Flavivirus RNA (sfRNA) element can comprise a recombinant or mutant sfRNA.

### 2. Vectors

Disclosed herein is a vector, comprising a disclosed isolated nucleic acid molecule. Disclosed herein is a vector, comprising an isolated nucleic acid molecule, comprising a nucleic acid sequence encoding a polypeptide or an RNA molecule, and a nucleic acid sequence encoding one or more Flavivirus genetic elements. Disclosed herein is a vector, comprising an isolated nucleic acid molecule, comprising a nucleic acid sequence encoding a polypeptide or an RNA molecule, and a nucleic acid sequence encoding one or more subgenomic Flavivirus RNA (sfRNA) elements.

Disclosed herein is a vector, comprising an isolated nucleic acid molecule, comprising a nucleic acid sequence encoding a polypeptide or an RNA molecule, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is a vector, comprising an isolated nucleic acid molecule, comprising a nucleic acid sequence encoding a polypeptide or an RNA molecule, and a nucleic acid sequence encoding a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is a vector, comprising an isolated nucleic acid molecule, comprising a nucleic acid sequence encoding a polypeptide or an RNA molecule, and a nucleic acid sequence encoding one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is a vector, comprising an isolated nucleic acid molecule, comprising a nucleic acid sequence encoding a polypeptide or an RNA molecule, and a nucleic acid sequence encoding one or more Flavivirus 3′ untranslated region (3′ UTR) elements.

Disclosed herein is a vector, comprising an isolated nucleic acid molecule, comprising a nucleic acid sequence encoding a polypeptide or an RNA molecule, and a nucleic acid sequence encoding one or more Flavivirus XRN1-resistant RNA (xrRNA) elements. In an aspect of a disclosed vector, a disclosed isolated nucleic acid molecule can further comprise one or more Flavivirus sfRNA dumbbell (DB) RNA elements, one or more Flavivirus 3′ stem loop (3′ SL) elements, or any combination thereof.

Disclosed herein is a vector, comprising an isolated nucleic acid molecule, comprising a nucleic acid sequence encoding a polypeptide or an RNA molecule, and a nucleic acid sequence encoding one or more Flavivirus sfRNA dumbbell (DB) RNA elements. In an aspect of a disclosed vector, a disclosed isolated nucleic acid molecule can further comprise one or more Flavivirus XRN1-resistant RNA (xrRNA) elements, one or more Flavivirus 3′ stem loop (3′ SL) elements, or any combination thereof.

Disclosed herein is a vector, comprising an isolated nucleic acid molecule, comprising a nucleic acid sequence encoding a polypeptide or an RNA molecule, and a nucleic acid sequence encoding one or more Flavivirus 3′ stem loop (3′ SL) elements. In an aspect of a disclosed vector, a disclosed isolated nucleic acid molecule can further comprise one or more Flavivirus sfRNA dumbbell (DB) RNA elements, one or more Flavivirus XRN1-resistant RNA (xrRNA) elements, or any combination thereof.

Disclosed herein is a vector, comprising an isolated nucleic acid molecule, comprising a nucleic acid sequence encoding a polypeptide or an RNA molecule, and a nucleic acid sequence encoding one or more 3′ UTRs of a Flavivirus, wherein the one or more 3′ UTRs comprise one or more subgenomic Flavivirus RNA (sfRNA) elements. In an aspect of a disclosed vector, a disclosed isolated nucleic acid molecule can further comprise one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is a vector, comprising an isolated nucleic acid molecule, comprising a nucleic acid sequence encoding a polypeptide or an RNA molecule, and a nucleic acid sequence encoding one or more 3′ UTRs of a Flavivirus, wherein the one or more 3′ UTRs comprises one or more xrRNA elements, one or more DB RNA elements, a 3′ stem loop, or any combination thereof.

Disclosed herein is a vector, comprising an isolated nucleic acid molecule, comprising a nucleic acid sequence encoding a polypeptide or an RNA molecule, and a nucleic acid sequence encoding at least two Flavivirus XRN1-resistant RNA (xrRNA) element, at least two Flavivirus sfRNA dumbbell (DB) RNA elements, and a Flavivirus 3′ stem loop (3′ SL) element.

Disclosed herein is a vector, comprising: a disclosed expression cassette comprising a disclosed nucleic acid sequence operably linked to one or more disclosed expression control elements and/or message stabilizing elements.

Disclosed herein is a vector, comprising a gene expression cassette comprising an isolated nucleic acid molecule comprising a nucleic acid sequence encoding a polypeptide or one or more RNA elements operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding one or more Flavivirus genetic elements, wherein the isolated nucleic acid molecule is operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding one or more subgenomic Flavivirus RNA (sfRNA) elements, wherein the isolated nucleic acid molecule is operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof, wherein the isolated nucleic acid molecule is operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding a Flavivirus 3′ untranslated region (3′ UTR) element, wherein the 3′ UTR comprises one or more Flavivirus XRN1-resistant RNA (xrRNA) elements; one or more Flavivirus sfRNA dumbbell (DB) RNA elements, a Flavivirus 3′ stem loop (3′ SL) element; or any combination thereof, wherein the isolated nucleic acid molecule is operably linked to one or more disclosed expression control elements and/or message stabilizing elements.

Disclosed here is a vector, comprising a gene expression cassette comprising an isolated nucleic acid molecule, comprising a nucleic acid sequence encoding a polypeptide or an RNA molecule; and a nucleic acid sequence encoding one or more Flavivirus XRN1-resistant RNA (xrRNA) elements, one or more Flavivirus sfRNA dumbbell (DB) RNA elements, one or more Flavivirus 3′ stem loop (3′ SL) elements; or any combination, wherein the isolated nucleic acid molecule is operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding one or more Flavivirus 3′ untranslated region (3′ UTR) elements, wherein the isolated nucleic acid molecule is operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding one or more Flavivirus XRN1-resistant RNA (xrRNA) elements. In an aspect of a disclosed vector, a disclosed isolated nucleic acid molecule can further comprise one or more Flavivirus sfRNA dumbbell (DB) RNA elements, one or more Flavivirus 3′ stem loop (3′ SL) elements, or any combination thereof, wherein the isolated nucleic acid molecule is operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding one or more Flavivirus sfRNA dumbbell (DB) RNA elements. In an aspect of a disclosed vector, a disclosed isolated nucleic acid molecule can further comprise one or more Flavivirus XRN1-resistant RNA (xrRNA) elements, one or more Flavivirus 3′ stem loop (3′ SL) elements, or any combination thereof, wherein the isolated nucleic acid molecule is operably linked to one or more disclosed expression control elements and/or message stabilizing elements.

Disclosed herein is vector, comprising a gene expression cassette comprising an isolated nucleic acid molecule, comprising a nucleic acid sequence encoding a polypeptide or an RNA molecule, and a nucleic acid sequence encoding one or more Flavivirus 3′ stem loop (3′ SL) elements. In an aspect of a disclosed vector, a disclosed isolated nucleic acid molecule can further comprise one or more Flavivirus sfRNA dumbbell (DB) RNA elements, one or more Flavivirus XRN1-resistant RNA (xrRNA) elements, or any combination thereof, wherein the isolated nucleic acid molecule is operably linked to one or more disclosed expression control elements and/or message stabilizing elements.

Disclosed herein is a vector, comprising a gene expression cassette comprising an nucleic acid molecule, comprising a nucleic acid sequence encoding a polypeptide or an RNA molecule; and a nucleic acid sequence encoding one or more 3′ UTRs of a Flavivirus, wherein the one or more 3′ UTRs comprise one or more subgenomic Flavivirus RNA (sfRNA) elements.

In an aspect of a disclosed vector, a disclosed isolated nucleic acid molecule can further comprise one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof, wherein the isolated nucleic acid molecule is operably linked to one or more disclosed expression control elements and/or message stabilizing elements.

Disclosed herein is a vector, comprising a gene expression cassette comprising an nucleic acid molecule, comprising a nucleic acid sequence encoding a polypeptide or an RNA molecule; and a nucleic acid sequence encoding one or more 3′ UTRs of a Flavivirus, wherein the one or more 3′ UTRs comprises one or more xrRNA elements, one or more DB RNA elements, a 3′ stem loop, or any combination thereof, wherein the isolated nucleic acid molecule is operably linked to one or more disclosed expression control elements and/or message stabilizing elements.

Disclosed herein is a vector, comprising a gene expression cassette comprising an isolated nucleic acid molecule, comprising a nucleic acid sequence encoding a polypeptide or an RNA molecule; and a nucleic acid sequence encoding at least two Flavivirus XRN1-resistant RNA (xrRNA) element, at least two Flavivirus sfRNA dumbbell (DB) RNA elements, and a Flavivirus 3′ stem loop (3′ SL) element, wherein the isolated nucleic acid molecule is operably linked to one or more disclosed expression control elements and/or message stabilizing elements.

In an aspect, a disclosed vector can restore one or more aspects of cellular homeostasis and/or cellular functionality and/or metabolic dysregulation. In an aspect, a disclosed vector can restore the functionality and/or structural integrity of a missing, deficient, and/or mutant protein or enzyme. In an aspect, a disclosed vector can increase the stability or half-life of an RNA molecule. In an aspect, a disclosed vector can increase the translation efficiency of an RNA molecule. In an aspect, a disclosed vector can enhance gene therapy.

In an aspect, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed nucleic acid sequence can have a coding sequence that is less than about 4.5 kilobases.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector. LNPs are discussed supra. In an aspect, a disclosed viral vector can be an adenovirus vector, an AAV vector, a herpes simplex virus vector, a retrovirus vector, an anelloviral vector, a lentivirus vector, and alphavirus vector, a Flavivirus vector, a rhabdovirus vector, a measles virus vector, a Newcastle disease viral vector, a poxvirus vector, or a picomavirus vector. Viral vectors are known to the art.

In an aspect, a disclosed viral vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Other engineered AAV variants include, but not limited to, AAV-DJ, AAV-HAE1, AAV-HAE2, AAVM41, AAV-1829, AAV1RX, CAM130, AAV2 Y/F, AAV2 T/V, AAV2i8, AAV2.5, AAV2G9, AAV8G9, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV.CAP-B10, AAV.MAC, AAVMyo, MYOAAV, AAV-F, AAV.cc44, AAV.cc47, AAV.cc81, and AAV.cc84. In an aspect, a disclosed AAV vector can be AAV-Rh74 or a related variant (e.g., capsid variants like RHM4-1). In an aspect, a disclosed AAV vector can be AAVhum.8.

In an aspect, a disclosed vector can comprise a promoter operably linked to a disclosed transgene. In an aspect, a disclosed promoter can be positioned 5′ (upstream) or 3′ (downstream) of a transgene under its control. The distance between the promoter and a transgene can be approximately the same as the distance between that promoter and the transgene it controls in the transgene from which the promoter is derived. As is known in the art, variation in this distance can be accommodated without loss of promoter function.

Depending on the level and tissue-specific expression desired, a variety of promoter elements can be used in a disclosed method. A promoter can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced.

In an aspect, a disclosed promoter can be a promoter/enhancer. In an aspect, a disclosed promoter can be an endogenous promoter. In an aspect, a disclosed endogenous promoter can be an endogenous promoter/enhancer. In an aspect, a disclosed endogenous promoter or a disclosed endogenous promoter/enhancer can generally be obtained from a non-coding region upstream of a transcription initiation site of a gene of interest. In an aspect, a disclosed endogenous promoter or a disclosed endogenous promoter/enhancer can be used for constitutive and efficient expression of a disclosed gene.

In an aspect, a disclosed vector can comprise a tissue-specific promoter operably linked to disclosed encoded polypeptide, a disclosed gene cassette, or a disclosed isolated nucleic acid molecule. “Tissue-specific promoters” are known to the art and are discussed supra and include, but are not limited to, neuron-specific promoters, muscle-specific promoters, liver-specific promoters, skeletal muscle-specific promoters, and heart-specific promoters.

In an aspect, a disclosed vector can comprise a ubiquitous/constitutive promoter operably linked to disclosed encoded polypeptide, a disclosed gene cassette, or a disclosed isolated nucleic acid molecule. “Ubiquitous/constitutive promoters” are known to the art and are discussed supra.

In an aspect, a disclosed vector can comprise an inducible promoter operably linked to disclosed encoded polypeptide, a disclosed gene cassette, or a disclosed isolated nucleic acid molecule. As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

In an aspect, a disclosed promoter can be a chicken R-actin promoter (CBA). In an aspect, a disclosed CBA promoter can comprise the sequence set forth in SEQ ID NO:36 or a fragment thereof. In an aspect, a disclosed CBA promoter can comprise a sequence having at least 50%, 55%, 60%, 65%, 70%, 75%, 80%, 85%, 86%, 87%, 88%, 89%, 90%, 91%, 92%, 93%, 94%, 95%, 96%, 97%, 98%, 99% identity to the sequence set forth in any SEQ ID NO:36.

In an aspect, a disclosed vector can comprise one or more CRISPR-based epigenome editing tools. In an aspect, a disclosed vector can comprise the sequence for one or more gRNAs. gRNAs are known to the art. In an aspect, a disclosed gRNA can target an endogenous gene. In an aspect, a disclosed vector can comprise a promoter operably linked to the one or more gRNAs.

In an aspect, a disclosed promoter operably linked to the one or more gRNAs can comprise a ubiquitous promoter, a constitutive promoter, or a tissue specific promoter. In an aspect, a disclosed promoter for the one or more gRNAs can comprise a U6 promoter.

### 3. Formulations

Disclosed herein is a pharmaceutical formulation comprising an isolated nucleic acid molecule comprising a nucleic acid sequence encoding a transgene and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof.

Disclosed herein is a pharmaceutical formulation comprising an isolated nucleic acid molecule comprising a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof.

Disclosed herein is a pharmaceutical formulation comprising a disclosed isolated nucleic acid molecule. Disclosed herein is a pharmaceutical formulation comprising a disclosed isolated nucleic acid molecule and a pharmaceutically acceptable carrier.

Disclosed herein is a pharmaceutical formulation comprising an isolated nucleic acid molecule, comprising one or more subgenomic Flavivirus RNA (sfRNA) elements. Disclosed herein is a pharmaceutical formulation comprising an isolated nucleic acid molecule, comprising one or more subgenomic Flavivirus RNA (sfRNA) elements and a pharmaceutically acceptable carrier.

Disclosed herein is a pharmaceutical formulation comprising an isolated nucleic acid molecule, comprising one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof. Disclosed herein is a pharmaceutical formulation comprising an isolated nucleic acid molecule, comprising one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof and a pharmaceutically acceptable carrier.

Disclosed herein is a pharmaceutical formulation comprising an isolated nucleic acid molecule, comprising a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is a pharmaceutical formulation comprising an isolated nucleic acid molecule, comprising one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is a pharmaceutical formulation comprising an isolated nucleic acid molecule, comprising one or more Flavivirus 3′ untranslated region (3′ UTR) elements.

Disclosed herein is a pharmaceutical formulation comprising an isolated nucleic acid molecule, comprising one or more Flavivirus XRN1-resistant RNA (xrRNA) elements; further comprising one or more Flavivirus sfRNA dumbbell (DB) RNA elements, one or more Flavivirus 3′ stem loop (3′ SL) elements, or any combination thereof.

Disclosed herein is a pharmaceutical formulation comprising an isolated nucleic acid molecule, comprising one or more Flavivirus sfRNA dumbbell (DB) RNA elements. Disclosed herein is a pharmaceutical formulation comprising an isolated nucleic acid molecule, comprising one or more Flavivirus 3′ stem loop (3′ SL) elements. Disclosed herein is a pharmaceutical formulation comprising an isolated nucleic acid molecule, comprising one or more 3′ UTRs of a Flavivirus, wherein the one or more 3′ UTRs comprise one or more subgenomic Flavivirus RNA (sfRNA) elements. Disclosed herein is a pharmaceutical formulation comprising an isolated nucleic acid molecule, comprising one or more 3′ UTRs of a Flavivirus, wherein the one or more 3′ UTRs comprises one or more xrRNA elements, one or more DB RNA elements, a 3′ stem loop, or any combination thereof.

Disclosed herein is a pharmaceutical formulation comprising an isolated nucleic acid molecule, comprising at least two Flavivirus XRN1-resistant RNA (xrRNA) element, at least two Flavivirus sfRNA dumbbell (DB) RNA elements, and a Flavivirus 3′ stem loop (3′ SL) element.

Disclosed herein is a pharmaceutical formulation comprising a disclosed vector.

Disclosed herein is a pharmaceutical formulation comprising a disclosed vector, comprising a disclosed isolated nucleic acid molecule.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding one or more Flavivirus genetic elements.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding one or more subgenomic Flavivirus RNA (sfRNA) elements.

Disclosed herein is a pharmaceutical formulation comprising a disclosed vector comprising a nucleic acid sequence encoding a polypeptide or an RNA molecule, and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding a Flavivirus 3′ untranslated region (3′ UTR) element, wherein the 3′ UTR comprises one or more Flavivirus XRN1-resistant RNA (xrRNA) elements, one or more Flavivirus sfRNA dumbbell (DB) RNA elements, a Flavivirus 3′ stem loop (3′ SL) element, or any combination thereof.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding one or more Flavivirus 3′ untranslated region (3′ UTR) elements.

Disclosed herein is a pharmaceutical formulation comprising a disclosed vector comprising a nucleic acid sequence encoding a polypeptide or an RNA molecule, and a nucleic acid sequence encoding one or more Flavivirus XRN1-resistant RNA (xrRNA) elements. In an aspect of a disclosed vector, a disclosed isolated nucleic acid molecule can further comprise one or more Flavivirus sfRNA dumbbell (DB) RNA elements, one or more Flavivirus 3′ stem loop (3′ SL) elements, or any combination thereof.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding one or more Flavivirus sfRNA dumbbell (DB) RNA elements. In an aspect of a disclosed vector, a disclosed isolated nucleic acid molecule can further comprise one or more Flavivirus XRN1-resistant RNA (xrRNA) elements, one or more Flavivirus 3′ stem loop (3′ SL) elements, or any combination thereof.

Disclosed herein is a pharmaceutical formulation comprising a disclosed vector comprising a nucleic acid sequence encoding a polypeptide or an RNA molecule, and a nucleic acid sequence encoding one or more Flavivirus 3′ stem loop (3′ SL) elements. In an aspect of a disclosed vector, a disclosed isolated nucleic acid molecule can further comprise one or more Flavivirus sfRNA dumbbell (DB) RNA elements, one or more Flavivirus XRN1-resistant RNA (xrRNA) elements, or any combination thereof.

Disclosed herein is a pharmaceutical formulation comprising a disclosed vector comprising a nucleic acid sequence encoding a polypeptide or an RNA molecule, and a nucleic acid sequence encoding one or more 3′ UTRs of a Flavivirus, wherein the one or more 3′ UTRs comprise one or more subgenomic Flavivirus RNA (sfRNA) elements. In an aspect of a disclosed vector, a disclosed isolated nucleic acid molecule can further comprise one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding one or more 3′ UTRs of a Flavivirus, wherein the one or more 3′ UTRs comprises one or more xrRNA elements, one or more DB RNA elements, a 3′ stem loop, or any combination thereof.

Disclosed herein is a pharmaceutical formulation comprising a disclosed vector comprising an isolated nucleic acid molecule, comprising a nucleic acid sequence encoding a polypeptide or an RNA molecule, and a nucleic acid sequence encoding at least two Flavivirus XRN1-resistant RNA (xrRNA) element, at least two Flavivirus sfRNA dumbbell (DB) RNA elements, and a Flavivirus 3′ stem loop (3′ SL) element.

Disclosed herein is a pharmaceutical formulation comprising a disclosed vector comprising a disclosed expression cassette comprising a disclosed nucleic acid sequence operably linked to one or more disclosed expression control elements and/or message stabilizing elements.

Disclosed herein is a pharmaceutical formulation comprising a disclosed vector comprising a gene expression cassette comprising an isolated nucleic acid molecule comprising a nucleic acid sequence encoding a polypeptide or one or more RNA elements operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule; and a nucleic acid sequence encoding one or more Flavivirus genetic elements, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule; and a nucleic acid sequence encoding one or more subgenomic Flavivirus RNA (sfRNA) elements, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule; and a nucleic acid sequence encoding one or more subgenomic Flavivirus RNA (sfRNA) elements, wherein the one or more sfRNA elements comprise a Flavivirus XRN1-resistant RNA (xrRNA) element, a Flavivirus sfRNA dumbbell (DB) RNA element, a Flavivirus 3′ stem loop (3′ SL) element, or any combination thereof, wherein the isolated nucleic acid molecule is operably linked to one or more disclosed expression control elements and/or message stabilizing elements.

Disclosed herein is a pharmaceutical formulation comprising a disclosed vector comprising an isolated nucleic acid molecule, comprising a nucleic acid sequence encoding a polypeptide or an RNA molecule; and a nucleic acid sequence encoding a Flavivirus 3′ untranslated region (3′ UTR) element, wherein the 3′ UTR comprises one or more Flavivirus XRN1-resistant RNA (xrRNA) elements; one or more Flavivirus sfRNA dumbbell (DB) RNA elements, a Flavivirus 3′ stem loop (3′ SL) element; or any combination thereof, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule; and a nucleic acid sequence encoding one or more Flavivirus XRN1-resistant RNA (xrRNA) elements, one or more Flavivirus sfRNA dumbbell (DB) RNA elements, one or more Flavivirus 3′ stem loop (3′ SL) elements; or any combination, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule; and a nucleic acid sequence encoding one or more Flavivirus 3′ untranslated region (3′ UTR) elements, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule; and a nucleic acid sequence encoding one or more Flavivirus XRN1-resistant RNA (xrRNA) elements. In an aspect of a disclosed vector, a disclosed isolated nucleic acid molecule can further comprise one or more Flavivirus sfRNA dumbbell (DB) RNA elements, one or more Flavivirus 3′ stem loop (3′ SL) elements, or any combination thereof, wherein the isolated nucleic acid molecule is operably linked to one or more disclosed expression control elements and/or message stabilizing elements.

Disclosed herein is a pharmaceutical formulation comprising a disclosed vector comprising an isolated nucleic acid molecule, comprising a nucleic acid sequence encoding a polypeptide or an RNA molecule; and a nucleic acid sequence encoding one or more Flavivirus sfRNA dumbbell (DB) RNA elements. In an aspect of a disclosed vector, a disclosed isolated nucleic acid molecule can further comprise one or more Flavivirus XRN1-resistant RNA (xrRNA) elements, one or more Flavivirus 3′ stem loop (3′ SL) elements, or any combination thereof, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule, and a nucleic acid sequence encoding one or more Flavivirus 3′ stem loop (3′ SL) elements. In an aspect of a disclosed vector, a disclosed isolated nucleic acid molecule can further comprise one or more Flavivirus sfRNA dumbbell (DB) RNA elements, one or more Flavivirus XRN1-resistant RNA (xrRNA) elements, or any combination thereof, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an nucleic acid molecule, comprising a nucleic acid sequence encoding a polypeptide or an RNA molecule; and a nucleic acid sequence encoding one or more 3′ UTRs of a Flavivirus, wherein the one or more 3′ UTRs comprise one or more subgenomic Flavivirus RNA (sfRNA) elements.

In an aspect of a disclosed vector, a disclosed isolated nucleic acid molecule can further comprise one or more Flavivirus XRN1-resistant RNA (xrRNA) elements, one or more Flavivirus sfRNA dumbbell (DB) RNA elements, one or more Flavivirus 3′ stem loop (3′ SL) elements, or any combination thereof, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an nucleic acid molecule, comprising a nucleic acid sequence encoding a polypeptide or an RNA molecule; and a nucleic acid sequence encoding one or more 3′ UTRs of a Flavivirus, wherein the one or more 3′ UTRs comprises one or more xrRNA elements, one or more DB RNA elements, a 3′ stem loop, or any combination thereof, wherein the isolated nucleic acid molecule is operably linked to one or more disclosed expression control elements and/or message stabilizing elements.

Disclosed herein is a pharmaceutical formulation comprising a disclosed vector comprising a gene expression cassette comprising an isolated nucleic acid molecule, comprising a nucleic acid sequence encoding a polypeptide or an RNA molecule; and a nucleic acid sequence encoding at least two Flavivirus XRN1-resistant RNA (xrRNA) element, at least two Flavivirus sfRNA dumbbell (DB) RNA elements, and a Flavivirus 3′ stem loop (3′ SL) element, wherein the isolated nucleic acid molecule is operably linked to one or more disclosed expression control elements and/or message stabilizing elements.

In an aspect, a disclosed pharmaceutical formulation can restore one or more aspects of cellular homeostasis and/or cellular functionality and/or metabolic dysregulation. In an aspect, a disclosed pharmaceutical formulation can restore the functionality and/or structural integrity of a missing, deficient, and/or mutant protein or enzyme.

In an aspect, a disclosed pharmaceutical formulation can comprise a vaccine.

In an aspect, a disclosed pharmaceutical formulation can increase the stability or half-life of an RNA molecule. In an aspect, a disclosed pharmaceutical formulation can increase the translation efficiency of an RNA molecule. In an aspect, a disclosed pharmaceutical formulation can enhance gene therapy.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more immune modulators. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof.

In an aspect, a disclosed formulation can comprise an anaplerotic agent (such as, for example, C7 compounds like triheptanoin or MCT).

In an aspect, a disclosed formulation can comprise an RNA therapeutic. An RNA therapeutic can comprise RNA-mediated interference (RNAi) and/or antisense oligonucleotides (ASO). In an aspect, a disclosed RNA therapeutic can be directed at any protein or enzyme that is overexpressed or is overactive due to a missing, deficient, and/or mutant protein or enzyme. In an aspect, a disclosed RNA therapeutic can be directed at any protein or enzyme that is overexpressed or is overactive due to vaccine. In an aspect, a disclosed RNA therapeutic can comprise therapy delivered via LNPs. In an aspect, a disclosed formulation can comprise an enzyme or enzyme precursor for enzyme replacement therapy (ERT).

In an aspect, a disclosed formulation can comprise a disclosed small molecule. In an aspect, a disclosed small molecule can assist in restoring the functionality and/or structural integrity of a missing, deficient, and/or mutant protein or enzyme.

In an aspect, any disclosed pharmaceutical formulation can comprise one or more excipients and/or pharmaceutically acceptable carriers.

### 4. Plasmids

Disclosed herein is a plasmid comprising one or more disclosed isolated nucleic acid molecules. Disclosed herein is a plasmid comprising one or more disclosed isolated nucleic acid molecules, wherein one or more disclosed isolated nucleic acid molecules can comprise a nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein is a plasmid comprising one or more disclosed vectors. Disclosed here are plasmids used in methods of making a disclosed composition such as, for example, a disclosed isolated nucleic acid molecule, a disclosed vector, or a disclosed pharmaceutical formulation.

Plasmids and using plasmids are known to the art.

### 5. Cells

Disclosed herein are cells comprising a disclosed isolated nucleic acid molecule, a disclosed vector, and/or a disclosed plasmid. Disclosed herein are cells comprising a disclosed isolated nucleic acid molecule, wherein a disclosed isolated nucleic acid molecule comprising a nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more XRN1-resistant RNA (xrRNA) elements, one or more Flavivirus sfRNA dumbbell (DB) RNA elements, one or more Flavivirus 3′ stem loop (3′ SL) elements, or any combination thereof.

Disclosed herein are cells transduced by a disclosed viral vector. Disclosed herein are cells transfected with a disclosed isolated nucleic acid molecule. Techniques to achieve transfection and transduction are known to the art and using transfected or transduced cells are known to the art. As known to the art, cell lines that can be transformed include carcinoma cell lines. Cell lines that can be used for viral vector production include but are not limited to HEK293 cells, HeLa cells, CHO, stem cell lines, fibroblasts, inducible pluripotent stem cells, primary airway cultures, primary kidney, primary cardiomyocytes, primary neurons, primary hepatocytes, primary myocytes or myotubes, kidney organoids, and brain organoids.

### 6. Animals

Disclosed herein are animals treated with one or more disclosed isolated nucleic acid molecules, one or more disclosed vectors, one or more disclosed pharmaceutical formulations, and/or one or more disclosed plasmids. Transgenic animals are known to the art as are the techniques to generate transgenic animals.

### C. Methods of Improving mRNA Transcript Stability

Disclosed herein is a method of improving mRNA transcript stability, comprising administering to a subject a therapeutically effective amount of a nucleic acid sequence encoding a transgene and an nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability.

Disclosed herein is a method of improving mRNA transcript stability, comprising administering to a subject a therapeutically effective amount of a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability.

Disclosed herein is a method of improving mRNA transcript stability, comprising administering to a subject a therapeutically effective amount of an isolated nucleic acid molecule encoding a transgene and encoding a Flavivirus 3′ untranslated region (3′ UTR) element, wherein expression of the nucleic acid molecule improves mRNA transcript stability.

Disclosed herein is a method of improving mRNA transcript stability, comprising administering to a subject a therapeutically effective amount of an isolated nucleic acid molecule encoding a transgene and encoding one or more Flavivirus genetic elements, wherein expression of the nucleic acid molecule improves mRNA transcript stability.

Disclosed herein is a method of improving mRNA transcript stability, comprising administering to a subject a therapeutically effective amount of an isolated nucleic acid molecule encoding a transgene and encoding one or more subgenomic Flavivirus RNA (sfRNA) elements, wherein expression of the nucleic acid molecule improves mRNA transcript stability.

Disclosed herein is a method of improving mRNA transcript stability, comprising administering to a subject a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript stability.

Disclosed herein is a method of improving mRNA transcript stability, comprising administering to a subject a therapeutically effective amount of a vector comprising an isolated nucleic acid molecule encoding a transgene and encoding one or more Flavivirus genetic elements, wherein expression of the nucleic acid molecule improves mRNA transcript stability.

Disclosed herein is a method of improving mRNA transcript stability, comprising administering to a subject a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript stability.

Disclosed herein is a method of improving mRNA transcript stability, comprising contacting one or more cells with a disclosed isolated nucleic acid molecule or a disclosed vector, wherein, following expression of the nucleic acid molecule, mRNA transcript stability is improved in the one or more cells. In an aspect, the one or more cells can be in a subject. In an aspect, a subject can have a disclosed genetic disease or disorder.

Disclosed herein is a method of improving mRNA transcript stability, comprising contacting one or more cells with a therapeutically effective amount of a nucleic acid sequence encoding a transgene and an nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability.

Disclosed herein is a method of improving mRNA transcript stability, comprising contacting one or more cells with a therapeutically effective amount of a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability.

Disclosed herein is a method of improving mRNA transcript stability, comprising contacting one or more cells with a therapeutically effective amount of an isolated nucleic acid molecule encoding a Flavivirus 3′ untranslated region (3′ UTR) element, wherein expression of the nucleic acid molecule improves mRNA transcript stability.

Disclosed herein is a method of improving mRNA transcript stability, comprising contacting one or more cells with a therapeutically effective amount of an isolated nucleic acid molecule encoding one or more Flavivirus genetic elements, wherein expression of the nucleic acid molecule improves mRNA transcript stability.

Disclosed herein is a method of improving mRNA transcript stability, comprising contacting one or more cells with a therapeutically effective amount of an isolated nucleic acid molecule encoding one or more subgenomic Flavivirus RNA (sfRNA) elements, wherein expression of the nucleic acid molecule improves mRNA transcript stability.

Disclosed herein is a method of improving mRNA transcript stability, comprising contacting one or more cells with a therapeutically effective amount of a vector comprising an isolated nucleic acid molecule encoding a Flavivirus 3′ untranslated region (3′ UTR) element, wherein expression of the nucleic acid molecule improves mRNA transcript stability.

Disclosed herein is a method of improving mRNA transcript stability, comprising contacting one or more cells with a therapeutically effective amount of a vector comprising an isolated nucleic acid molecule encoding one or more Flavivirus genetic elements, wherein expression of the nucleic acid molecule improves mRNA transcript stability.

Disclosed herein is a method of improving mRNA transcript stability, comprising contacting one or more cells with a therapeutically effective amount of a vector comprising an isolated nucleic acid molecule encoding one or more subgenomic Flavivirus RNA (sfRNA) elements, wherein expression of the nucleic acid molecule improves mRNA transcript stability.

Disclosed herein is a method of improving mRNA transcript stability, comprising contacting one or more cells with a disclosed isolated nucleic acid molecule or a disclosed vector, wherein, following expression of the nucleic acid molecule, mRNA transcript stability is improved in the one or more cells. In an aspect, the one or more cells can be in a subject. In an aspect, a subject can have a disclosed genetic disease or disorder.

In an aspect, a disclosed method of improving mRNA transcript stability can be used in vaccine formulation.

In an aspect of a method of improving mRNA transcript stability, a transgene can comprise any gene disclosed herein (discussed supra in connection with the disclosed Compositions) or any gene that is missing, deficient, and/or mutated or any gene that is producing a deficient or mutated protein or enzyme. By knowing what disease or disorder is affecting the subject, the skilled person can identify the relevant gene or genes.

In an aspect, a subject can have a disease or disorder. In an aspect, a disease or disorder can be any disease or disorder disclosed herein. In an aspect, a disease or disorder can comprise any disease or disorder caused by a disclosed gene or a missing, deficient, and/or mutant gene. In an aspect, a subject can be a subject in need of treatment of a disclosed disease or disorder (e.g., a genetic disease or disorder).

In an aspect, a subject can have or be suspected of having a disease or disorder that can be treated with gene therapy. Examples of such diseases or disorder can include, but are not limited to: cystic fibrosis (cystic fibrosis transmembrane regulator protein) and other diseases of the lung, hemophilia A (Factor VIII), hemophilia B (Factor IX), thalassemia (0-globin), anemia (erythropoietin) and other blood disorders, Alzheimer's disease (GDF; neprilysin), multiple sclerosis (0-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the I-kappa B dominant mutant, sarcospan, utrophin, mini-utrophin, antisense or RNAi against splice junctions in the dystrophin gene to induce exon skipping (see, e.g., WO 2003/095647), antisense against U7 snRNAs to induce exon skipping (see, e.g., WO 2006/021724), and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic disorders, congenital emphysema (a1-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (1-1) and fragments thereof (e.g., IIC), serca2a, zinc finger proteins that regulate the phospholamban gene, Barkct, P2-adrenergic receptor, p2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFa soluble receptor), hepatitis (α-interferon), LDL receptor deficiency (LDL receptor), hyperammonemia (omithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like.

In an aspect, a disclosed method can restore the functionality and/or structural integrity of a missing, deficient, and/or mutant protein or enzyme. In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of improving mRNA transcript stability, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are provided below.

| Regulated Variable | Sensor |
|---|---|
| Blood Pressure/Blood | Aortic Body (Aorta), Carotid |
| Volume/Na⁺ Conc./ | Body (Carotid Artery), Atrial |
| | Volume Receptors (Heart), |
| | Juxtaglomerular Apparatus (Kidney) |
| Ca²⁺/Mg²⁺/PO₄³⁻ Conc. | Chief Cells (Parathyroid Gland) |
| Glucose | Islet of Langerhans (Pancreas) |
| Osmolarity | Circumventricular Organs |
| | (Hypothalamus) |
| pO₂, pCO₂, and pH | Aortic Body (Aorta), Carotid |
| | Body (Carotid Artery), |
| | Ventrolateral Medulla (Medulla) |
| Temperature | Thermosensory neurons (Skin), |
| | Preoptic Area (Hypothalamus) |

In an aspect of a disclosed method, administering can comprise intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed nucleic acid molecule, a disclosed vector, and/or a disclosed pharmaceutical formulation can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed nucleic acid molecule, a disclosed vector, and/or a disclosed pharmaceutical formulation can comprise IV administration and intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect, a disclosed vector can be administered via LNP administration. Lipid nanoparticles (LNPs) are a well-known means for delivery of nucleotide and protein cargo and can be used for delivery of one or more disclosed nucleic acids molecules.

In an aspect of a method of improving mRNA transcript stability, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method can further comprise administering to the subject a therapeutically effective amount of a therapeutic agent. A therapeutic agent can be any disclosed agent that effects a desired clinical outcome.

In an aspect, a disclosed method of improving mRNA transcript stability can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect, a disclosed method of improving mRNA transcript stability can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes. Cas9 can have the amino acid sequence set forth in SEQ ID NO:34, SEQ ID NO:35, or a fragment thereof. In an aspect, a disclosed Cas9 can have a sequence having at least 75%, at least 80%, at least 85%, at least 90%, or at least 95% identity to the amino acid sequence set forth in SEQ ID NO:34, SEQ ID NO:35, or a fragment thereof. In an aspect, a disclosed nucleic acid sequence for Cas9 can comprise the sequence set forth in SEQ ID NO:33 or a fragment thereof. In an aspect, a disclosed nucleic acid sequence for Cas9 can comprise a sequence having at least 80%, at least 85%, at least 90%, or at least 95% identity to the sequence set forth in SEQ ID NO:33 or a fragment thereof. In an aspect, a disclosed method can comprise administering the subject a disclosed RNA therapeutic.

In an aspect, a disclosed method of improving mRNA transcript stability can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of improving mRNA transcript stability can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of improving mRNA transcript stability can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of improving mRNA transcript stability can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of improving mRNA transcript stability can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted. In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of improving mRNA transcript stability can further comprise treating a subject that has developed or is likely to develop neutralizing antibodies (ABs) to a disclosed vector, a disclosed capsid, and/or a disclosed transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method can comprise modifying one or more of the disclosed steps. For example, modifying one or more of steps of a disclosed method can comprise modifying or changing one or more features or aspects of one or more steps of a disclosed method.

For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a method can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed isolated nucleic acid molecules, one or more disclosed vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of improving mRNA transcript stability can further comprise generating a disclosed isolated nucleic acid molecule. In an aspect, a disclosed method can further comprise generating a disclosed viral or non-viral vector. In an aspect, generating a disclosed viral vector can comprise generating an AAV vector or a recombinant AAV (such as those disclosed herein).

### D. Methods of Improving mRNA Transcript Translation Efficiency

Disclosed herein is a method of improving mRNA transcript translation efficiency, comprising administering to a subject a therapeutically effective amount of an isolated nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof.

Disclosed herein is a method of improving mRNA transcript translation efficiency, comprising administering to a subject a therapeutically effective amount of a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof.

Disclosed herein is a method of improving mRNA transcript translation efficiency, comprising administering to a subject a therapeutically effective amount of an isolated nucleic acid molecule encoding a transgene and encoding a Flavivirus 3′ untranslated region (3′ UTR) element, wherein expression of the nucleic acid molecule improves mRNA transcript translation efficiency.

Disclosed herein is a method of improving mRNA transcript translation efficiency, comprising administering to a subject a therapeutically effective amount of an isolated nucleic acid molecule encoding a Flavivirus 3′ untranslated region (3′ UTR) element, wherein expression of the nucleic acid molecule improves mRNA transcript translation efficiency.

Disclosed herein is a method of improving mRNA transcript translation efficiency, comprising administering to a subject a therapeutically effective amount of an isolated nucleic acid molecule encoding a transgene and encoding one or more Flavivirus genetic elements, wherein expression of the nucleic acid molecule improves mRNA transcript translation efficiency.

Disclosed herein is a method of improving mRNA transcript translation efficiency, comprising administering to a subject a therapeutically effective amount of an isolated nucleic acid molecule encoding a transgene and encoding one or more subgenomic Flavivirus RNA (sfRNA) elements, wherein expression of the nucleic acid molecule improves mRNA transcript translation efficiency.

Disclosed herein is a method of improving mRNA transcript translation efficiency, comprising administering to a subject a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript translation efficiency.

Disclosed herein is a method of improving mRNA transcript translation efficiency, comprising administering to a subject a therapeutically effective amount of a vector comprising an isolated nucleic acid molecule encoding a transgene and encoding one or more Flavivirus genetic elements, wherein expression of the nucleic acid molecule improves mRNA transcript translation efficiency.

Disclosed herein is a method of improving mRNA transcript translation efficiency, comprising administering to a subject a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript translation efficiency.

Disclosed herein is a method of improving mRNA transcript translation efficiency, comprising contacting one or more cells with a disclosed isolated nucleic acid molecule or a disclosed vector, wherein, following expression of the nucleic acid molecule, mRNA transcript translation efficiency in the one or more cells is improved. In an aspect, the one or more cells can be in a subject. In an aspect, a subject can have a disclosed genetic disease or disorder.

Disclosed herein is a method of improving mRNA transcript translation efficiency, comprising contacting one or more cells with a therapeutically effective amount of an isolated nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof.

Disclosed herein is a method of improving mRNA transcript translation efficiency, comprising contacting one or more cells with a therapeutically effective amount of a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof.

Disclosed herein is a method of improving mRNA transcript translation efficiency, comprising contacting one or more cells with a therapeutically effective amount of an isolated nucleic acid molecule encoding a transgene and encoding a Flavivirus 3′ untranslated region (3′ UTR) element, wherein expression of the nucleic acid molecule improves mRNA transcript translation efficiency.

Disclosed herein is a method of improving mRNA transcript translation efficiency, comprising contacting one or more cells with a therapeutically effective amount of an isolated nucleic acid molecule encoding a Flavivirus 3′ untranslated region (3′ UTR) element, wherein expression of the nucleic acid molecule improves mRNA transcript translation efficiency.

Disclosed herein is a method of improving mRNA transcript translation efficiency, comprising contacting one or more cells with a therapeutically effective amount of an isolated nucleic acid molecule encoding a transgene and encoding one or more Flavivirus genetic elements, wherein expression of the nucleic acid molecule improves mRNA transcript translation efficiency.

Disclosed herein is a method of improving mRNA transcript translation efficiency, comprising contacting one or more cells with a therapeutically effective amount of an isolated nucleic acid molecule encoding a transgene and encoding one or more subgenomic Flavivirus RNA (sfRNA) elements, wherein expression of the nucleic acid molecule improves mRNA transcript translation efficiency.

Disclosed herein is a method of improving mRNA transcript translation efficiency, comprising contacting one or more cells with a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript translation efficiency.

Disclosed herein is a method of improving mRNA transcript translation efficiency, comprising contacting one or more cells with a therapeutically effective amount of a vector comprising an isolated nucleic acid molecule encoding a transgene and encoding one or more Flavivirus genetic elements, wherein expression of the nucleic acid molecule improves mRNA transcript translation efficiency.

Disclosed herein is a method of improving mRNA transcript translation efficiency, comprising contacting one or more cells with a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript translation efficiency.

Disclosed herein is a method of improving mRNA transcript translation efficiency, comprising contacting one or more cells with a disclosed isolated nucleic acid molecule or a disclosed vector, wherein, following expression of the nucleic acid molecule, mRNA transcript translation efficiency in the one or more cells is improved. In an aspect, the one or more cells can be in a subject. In an aspect, a subject can have a disclosed genetic disease or disorder.

In an aspect, a disclosed method of improving mRNA transcript translation efficiency can be used in vaccine formulation.

In an aspect of a method of improving mRNA transcript translation efficiency, a transgene can comprise any gene disclosed herein (discussed supra in connection with the disclosed Compositions) or any gene that is missing, deficient, and/or mutated or any gene that is producing a deficient or mutated protein or enzyme. By knowing what disease or disorder is affecting the subject, the skilled person can identify the relevant gene or genes.

In an aspect, a subject can be a subject having a disease or disorder. In an aspect, a disease or disorder can be any disease or disorder disclosed herein (and discussed supra in connection with the methods of improving mRNA transcript). In an aspect, a disease or disorder can comprise any disease or disorder caused by a disclosed gene. In an aspect, a subject can be a subject in need of treatment of a disclosed disease or disorder (e.g., a genetic disease or disorder).

In an aspect, a disclosed method can restore the functionality and/or structural integrity of a missing, deficient, and/or mutant protein or enzyme. In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of improving mRNA transcript translation efficiency, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method, administering can comprise intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed vector can be administered via LNP administration. In an aspect, a disclosed nucleic acid molecule, a disclosed vector, and/or a disclosed pharmaceutical formulation can be concurrently and/or serially administered to a subject via multiple routes of administration.

For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed nucleic acid molecule, a disclosed vector, and/or a disclosed pharmaceutical formulation can comprise IV administration and intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect of a method of improving mRNA transcript translation efficiency, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method can further comprise administering to the subject a therapeutically effective amount of a therapeutic agent. A therapeutic agent can be any disclosed agent that effects a desired clinical outcome.

In an aspect, a disclosed method of improving mRNA transcript translation efficiency can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect, a disclosed method of improving mRNA transcript translation efficiency can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes. Cas9 can have the amino acid sequence set forth in SEQ ID NO:34, SEQ ID NO:35, or a fragment thereof. In an aspect, a disclosed Cas9 can have a sequence having at least 75%, at least 80%, at least 85%, at least 90%, or at least 95% identity to the amino acid sequence set forth in SEQ ID NO:34, SEQ ID NO:35, or a fragment thereof. In an aspect, a disclosed nucleic acid sequence for Cas9 can comprise the sequence set forth in SEQ ID NO:33 or a fragment thereof. In an aspect, a disclosed nucleic acid sequence for Cas9 can comprise a sequence having at least 80%, at least 85%, at least 90%, or at least 95% identity to the sequence set forth in SEQ ID NO:33 or a fragment thereof. In an aspect, a disclosed method can comprise administering the subject a disclosed RNA therapeutic.

In an aspect, a disclosed method of improving mRNA transcript translation efficiency can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of improving mRNA transcript translation efficiency can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of improving mRNA transcript translation efficiency can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of improving mRNA transcript translation efficiency can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of improving mRNA transcript translation efficiency can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted. In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of improving mRNA transcript translation efficiency can further comprise treating a subject that has developed or is likely to develop neutralizing antibodies (ABs) to a disclosed vector, a disclosed capsid, and/or a disclosed transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method can comprise modifying one or more of the disclosed steps. For example, modifying one or more of steps of a disclosed method can comprise modifying or changing one or more features or aspects of one or more steps of a disclosed method.

For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a disclosed method of improving mRNA transcript translation efficiency can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed isolated nucleic acid molecules, one or more disclosed vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of improving mRNA transcript translation efficiency can further comprise generating a disclosed isolated nucleic acid molecule. In an aspect, a disclosed method can further comprise generating a disclosed viral or non-viral vector. In an aspect, generating a disclosed viral vector can comprise generating an AAV vector or a recombinant AAV (such as those disclosed herein).

### E. Methods of Enhancing Gene Therapy

Disclosed herein is a method of enhancing gene therapy, comprising administering to a subject in need thereof a therapeutically effective amount of an isolated nucleic acid molecule comprising a nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of an isolated nucleic acid molecule comprising a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of an isolated nucleic acid molecule encoding a transgene and encoding a Flavivirus 3′ untranslated region (3′ UTR) element, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of an isolated nucleic acid molecule encoding a transgene and encoding one or more Flavivirus genetic elements, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of an isolated nucleic acid molecule encoding a transgene and encoding one or more subgenomic Flavivirus RNA (sfRNA) elements, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of a vector comprising an isolated nucleic acid molecule encoding a transgene and encoding one or more Flavivirus genetic elements, wherein expression of the nucleic acid molecule improves mRNA transcript stability and/or improves mRNA transcript translation efficiency.

Disclosed herein is a method of enhancing gene therapy, comprising administering to a subject in need thereof a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript stability and/or improves mRNA transcript translation efficiency.

Disclosed herein is a method of enhancing gene therapy, comprising contacting one or more cells with a therapeutically effective amount of an isolated nucleic acid molecule comprising a nucleic acid sequence encoding a transgene; and a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of enhancing gene therapy, comprising contacting one or more cells with a therapeutically effective amount of an isolated nucleic acid molecule comprising a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of enhancing gene therapy, comprising contacting one or more cells with a therapeutically effective amount of an isolated nucleic acid molecule encoding a transgene and encoding a Flavivirus 3′ untranslated region (3′ UTR) element, wherein expression of the nucleic acid molecule improves mRNA transcript stability and/or improves mRNA transcript translation efficiency.

Disclosed herein is a method of enhancing gene therapy, comprising contacting one or more cells with a therapeutically effective amount of an isolated nucleic acid molecule encoding a transgene and encoding one or more Flavivirus genetic elements, wherein expression of the nucleic acid molecule improves mRNA transcript stability and/or improves mRNA transcript translation efficiency.

Disclosed herein is a method of enhancing gene therapy, comprising contacting one or more cells with a therapeutically effective amount of an isolated nucleic acid molecule encoding a transgene and encoding one or more subgenomic Flavivirus RNA (sfRNA) elements, wherein expression of the nucleic acid molecule improves mRNA transcript stability and/or improves mRNA transcript translation efficiency.

Disclosed herein is a method of enhancing gene therapy, contacting one or more cells with a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript stability and/or improves mRNA transcript translation efficiency.

Disclosed herein is a method of enhancing gene therapy, comprising contacting one or more cells with a therapeutically effective amount of a vector comprising an isolated nucleic acid molecule encoding a transgene and encoding one or more Flavivirus genetic elements, wherein expression of the nucleic acid molecule improves mRNA transcript stability and/or improves mRNA transcript translation efficiency.

Disclosed herein is a method of enhancing gene therapy, comprising contacting one or more cells with a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript stability and/or improves mRNA transcript translation efficiency. In an aspect, the one or more cells can be in a subject. In an aspect, a subject can have a disclosed genetic disease or disorder.

In an aspect, a disclosed method of enhancing gene therapy can be used in vaccine formulation.

In an aspect of a method of enhancing gene therapy, a transgene can comprise any gene disclosed herein (discussed supra in connection with the disclosed Compositions) or any gene that is missing, deficient, and/or mutated or any gene that is producing a deficient or mutated protein or enzyme. By knowing what disease or disorder is affecting the subject, the skilled person can identify the relevant gene or genes.

In an aspect, a subject can be a subject having a disease or disorder. In an aspect, a disease or disorder can be any disease or disorder disclosed herein (and discussed supra in connection with the methods of improving mRNA transcript stability). In an aspect, a disease or disorder can comprise any disease or disorder caused by a disclosed gene. In an aspect, a subject can be a subject in need of treatment of a disclosed disease or disorder (e.g., a genetic disease or disorder).

In an aspect, a disclosed method can restore the functionality and/or structural integrity of a missing, deficient, and/or mutant protein or enzyme. In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of enhancing gene therapy, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method, administering can comprise intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system.

In an aspect, a disclosed vector can be administered via LNP administration. In an aspect, a disclosed nucleic acid molecule, a disclosed vector, and/or a disclosed pharmaceutical formulation can be concurrently and/or serially administered to a subject via multiple routes of administration.

For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed nucleic acid molecule, a disclosed vector, and/or a disclosed pharmaceutical formulation can comprise IV administration and intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect of a method of enhancing gene therapy, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method can further comprise administering to the subject a therapeutically effective amount of a therapeutic agent. A therapeutic agent can be any disclosed agent that effects a desired clinical outcome.

In an aspect, a disclosed method of enhancing gene therapy can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect, a disclosed method of enhancing gene therapy can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes. Cas9 can have the amino acid sequence set forth in SEQ ID NO:34, SEQ ID NO:35, or a fragment thereof. In an aspect, a disclosed Cas9 can have a sequence having at least 75%, at least 80%, at least 85%, at least 90%, or at least 95% identity to the amino acid sequence set forth in SEQ ID NO:34, SEQ ID NO:35, or a fragment thereof. In an aspect, a disclosed nucleic acid sequence for Cas9 can comprise the sequence set forth in SEQ ID NO:33 or a fragment thereof. In an aspect, a disclosed nucleic acid sequence for Cas9 can comprise a sequence having at least 80%, at least 85%, at least 90%, or at least 95% identity to the sequence set forth in SEQ ID NO:33 or a fragment thereof. In an aspect, a disclosed method can comprise administering the subject a disclosed RNA therapeutic.

In an aspect, a disclosed method of enhancing gene therapy can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of enhancing gene therapy can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of enhancing gene therapy can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of enhancing gene therapy can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of enhancing gene therapy can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted. In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of enhancing gene therapy can further comprise treating a subject that has developed or is likely to develop neutralizing antibodies (ABs) to a disclosed vector, a disclosed capsid, and/or a disclosed transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a disclosed method of enhancing gene therapy can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed isolated nucleic acid molecules, one or more disclosed vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of enhancing gene therapy can further comprise generating a disclosed isolated nucleic acid molecule. In an aspect, a disclosed method can further comprise generating a disclosed viral or non-viral vector. In an aspect, generating a disclosed viral vector can comprise generating an AAV vector or a recombinant AAV (such as those disclosed herein).

### F. Methods of Treating and/or Preventing a Disease or Disorder

Disclosed herein is a method of treating and/or preventing a disease or disorder, comprising enhancing gene therapy by administering to a subject in need thereof a therapeutically effective amount of an isolated nucleic acid molecule encoding a transgene and encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of treating and/or preventing a disease or disorder, comprising enhancing gene therapy by administering to a subject in need thereof a therapeutically effective amount of an isolated nucleic acid molecule encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of treating and/or preventing a disease or disorder, comprising enhancing gene therapy by administering to a subject in need thereof a therapeutically effective amount of an isolated nucleic acid molecule encoding a transgene and encoding a Flavivirus 3′ untranslated region (3′ UTR) element, wherein expression of the nucleic acid molecule improves mRNA transcript stability and/or improves mRNA transcript translation efficiency.

Disclosed herein is a method of treating and/or preventing a disease or disorder, comprising enhancing gene therapy by administering to a subject in need thereof a therapeutically effective amount of an isolated nucleic acid molecule encoding a transgene and encoding one or more Flavivirus genetic elements, wherein expression of the nucleic acid molecule improves mRNA transcript stability and/or improves mRNA transcript translation efficiency.

Disclosed herein is a method of treating and/or preventing a disease or disorder, comprising enhancing gene therapy by administering to a subject in need thereof a therapeutically effective amount of an isolated nucleic acid molecule encoding a transgene and encoding one or more subgenomic Flavivirus RNA (sfRNA) elements, wherein expression of the nucleic acid molecule improves mRNA transcript stability and/or improves mRNA transcript translation efficiency.

Disclosed herein is a method of treating and/or preventing a disease or disorder, comprising administering to a subject in need thereof a therapeutically effective amount of an isolated nucleic acid molecule encoding a transgene and encoding a Flavivirus 3′ untranslated region (3′ UTR) element, and enhancing gene therapy through improved mRNA transcript stability and/or improved mRNA transcript translation efficiency.

Disclosed herein is a method of treating and/or preventing a disease or disorder, comprising administering to a subject in need thereof a therapeutically effective amount of an isolated nucleic acid molecule encoding a transgene and encoding one or more Flavivirus genetic elements, and enhancing gene therapy through improved mRNA transcript stability and/or improved mRNA transcript translation efficiency.

Disclosed herein is a method of treating and/or preventing a disease or disorder, comprising administering to a subject in need thereof a therapeutically effective amount of an isolated nucleic acid molecule encoding a transgene and encoding one or more subgenomic Flavivirus RNA (sfRNA) elements, and enhancing gene therapy through improved mRNA transcript stability and/or improved mRNA transcript translation efficiency.

Disclosed herein is a method of treating and/or preventing a disease or disorder, comprising enhancing gene therapy by administering to a subject in need thereof a therapeutically effective amount of a vector comprising an isolated nucleic acid molecule encoding a transgene and encoding a Flavivirus 3′ untranslated region (3′ UTR) element, wherein expression of the nucleic acid molecule improves mRNA transcript stability and/or improves mRNA transcript translation efficiency.

Disclosed herein is a method of treating and/or preventing a disease or disorder, comprising enhancing gene therapy by administering to a subject in need thereof a therapeutically effective amount of a vector comprising an isolated nucleic acid molecule encoding a transgene and encoding one or more Flavivirus genetic elements, wherein expression of the nucleic acid molecule improves mRNA transcript stability and/or improves mRNA transcript translation efficiency.

Disclosed herein is a method of treating and/or preventing a disease or disorder, comprising enhancing gene therapy by administering to a subject in need thereof a therapeutically effective amount of a vector comprising an isolated nucleic acid molecule encoding a transgene and encoding one or more subgenomic Flavivirus RNA (sfRNA) elements, wherein expression of the nucleic acid molecule improves mRNA transcript A stability and/or improves mRNA transcript translation efficiency.

Disclosed herein is a method of treating and/or preventing a disease or disorder, comprising administering to a subject in need thereof a therapeutically effective amount of a vector comprising an isolated nucleic acid molecule encoding a transgene and encoding a Flavivirus 3′ untranslated region (3′ UTR) element, and enhancing gene therapy through improved mRNA transcript stability and/or improved mRNA transcript translation efficiency.

Disclosed herein is a method of treating and/or preventing a disease or disorder, comprising administering to a subject in need thereof a therapeutically effective amount a vector comprising of an isolated nucleic acid molecule encoding a transgene and encoding one or more Flavivirus genetic elements, and enhancing gene therapy through improved mRNA transcript stability and/or improved mRNA transcript translation efficiency.

Disclosed herein is a method of treating and/or preventing a disease or disorder, comprising administering to a subject in need thereof a therapeutically effective amount of a vector comprising an isolated nucleic acid molecule encoding a transgene and encoding one or more subgenomic Flavivirus RNA (sfRNA) elements, and enhancing gene therapy through improved mRNA transcript stability and/or improved mRNA transcript translation efficiency.

In an aspect of a method of treating and/or preventing a disease or disorder, a transgene can comprise any gene disclosed herein (discussed supra in connection with the disclosed Compositions) or any gene that is missing, deficient, and/or mutated or any gene that is producing a deficient or mutated protein or enzyme. By knowing what disease or disorder is affecting the subject, the skilled person can identify the relevant gene or genes.

In an aspect, a subject can be a subject having a disease or disorder. In an aspect, a disease or disorder can be any disease or disorder disclosed herein (and discussed supra in connection with the methods of improving mRNA transcript stability). In an aspect, a disease or disorder can comprise any disease or disorder caused by a disclosed gene. In an aspect, a subject can be a subject in need of treatment of a disclosed disease or disorder (e.g., a genetic disease or disorder).

In an aspect, a disclosed method can restore the functionality and/or structural integrity of a missing, deficient, and/or mutant protein or enzyme. In an aspect, a disclosed method can comprise restoring one or more aspects of cellular homeostasis and/or cellular functionality and/or metabolic dysregulation. In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of treating and/or preventing a disease or disorder, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect of a disclosed method, administering can comprise intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed vector can be administered via LNP administration. In an aspect, a disclosed nucleic acid molecule, a disclosed vector, and/or a disclosed pharmaceutical formulation can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed nucleic acid molecule, a disclosed vector, and/or a disclosed pharmaceutical formulation can comprise IV administration and intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect of a disclosed method of treating and/or preventing a disease or disorder, a therapeutically effective amount of disclosed vector can be delivered via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method can further comprise administering to the subject a therapeutically effective amount of a therapeutic agent. A therapeutic agent can be any disclosed agent that effects a desired clinical outcome.

In an aspect, a disclosed method of treating and/or preventing a disease or disorder can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

In an aspect, a disclosed method of treating and/or preventing a disease or disorder can comprise administering an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes. Cas9 can have the amino acid sequence set forth in SEQ ID NO:34, SEQ ID NO:35, or a fragment thereof. In an aspect, a disclosed Cas9 can have a sequence having at least 75%, at least 80%, at least 85%, at least 90%, or at least 95% identity to the amino acid sequence set forth in SEQ ID NO:34, SEQ ID NO:35, or a fragment thereof. In an aspect, a disclosed nucleic acid sequence for Cas9 can comprise the sequence set forth in SEQ ID NO:33 or a fragment thereof. In an aspect, a disclosed nucleic acid sequence for Cas9 can comprise a sequence having at least 80%, at least 85%, at least 90%, or at least 95% identity to the sequence set forth in SEQ ID NO:33 or a fragment thereof. In an aspect, a disclosed method can comprise administering the subject a disclosed RNA therapeutic.

In an aspect, a disclosed method of treating and/or preventing a disease or disorder can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of treating and/or preventing a disease or disorder can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of treating and/or preventing a disease or disorder can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of treating and/or preventing a disease or disorder can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of treating and/or preventing a disease or disorder can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted. In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of treating and/or preventing a disease or disorder can further comprise treating a subject that has developed or is likely to develop neutralizing antibodies (ABs) to a disclosed vector, a disclosed capsid, and/or a disclosed transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method can comprise modifying one or more of the disclosed steps. For example, modifying one or more of steps of a disclosed method can comprise modifying or changing one or more features or aspects of one or more steps of a disclosed method.

For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a disclosed method of treating and/or preventing a disease or disorder can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed isolated nucleic acid molecules, one or more disclosed vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of treating and/or preventing a disease or disorder can further comprise generating a disclosed isolated nucleic acid molecule. In an aspect, a disclosed method can further comprise generating a disclosed viral or non-viral vector. In an aspect, generating a disclosed viral vector can comprise generating an AAV vector or a recombinant AAV (such as those disclosed herein).

### G. Methods of Improving Vaccine Formulations

Disclosed herein is a method of improving vaccine formulation, comprising contacting one or more cells with a therapeutically effective amount of a nucleic acid sequence encoding a transgene and an nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of improving vaccine formulation, comprising contacting one or more cells with a therapeutically effective amount of a nucleic acid sequence encoding one or more Flavivirus genetic elements, wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof, wherein expression of the nucleic acid molecule improves mRNA transcript stability and/or improves mRNA transcript translation efficiency.

Disclosed herein is a method of improving vaccine formulation, comprising contacting one or more cells with a therapeutically effective amount of an isolated nucleic acid molecule encoding a Flavivirus 3′ untranslated region (3′ UTR) element, wherein expression of the nucleic acid molecule improves mRNA transcript stability and/or improves mRNA transcript translation efficiency.

Disclosed herein is a method of improving vaccine formulation, comprising contacting one or more cells with a therapeutically effective amount of an isolated nucleic acid molecule encoding one or more Flavivirus genetic elements, wherein expression of the nucleic acid molecule improves mRNA transcript stability and/or improves mRNA transcript translation efficiency.

Disclosed herein is a method of improving vaccine formulation, comprising contacting one or more cells with a therapeutically effective amount of an isolated nucleic acid molecule encoding one or more subgenomic Flavivirus RNA (sfRNA) elements, wherein expression of the nucleic acid molecule improves mRNA transcript stability and/or improves mRNA transcript translation efficiency.

Disclosed herein is a method of improving vaccine formulation, comprising contacting one or more cells with a therapeutically effective amount of a vector comprising an isolated nucleic acid molecule encoding a Flavivirus 3′ untranslated region (3′ UTR) element, wherein expression of the nucleic acid molecule improves mRNA transcript stability and/or improves mRNA transcript translation efficiency.

Disclosed herein is a method of improving vaccine formulation, comprising contacting one or more cells with a therapeutically effective amount of a vector comprising an isolated nucleic acid molecule encoding one or more Flavivirus genetic elements, wherein expression of the nucleic acid molecule improves mRNA transcript stability and/or improves mRNA transcript translation efficiency.

Disclosed herein is a method of improving vaccine formulation, comprising contacting one or more cells with a therapeutically effective amount of a vector comprising an isolated nucleic acid molecule encoding one or more subgenomic Flavivirus RNA (sfRNA) elements, wherein expression of the nucleic acid molecule improves mRNA transcript stability and/or improves mRNA transcript translation efficiency.

Disclosed herein is a method of improving vaccine formulation, comprising contacting one or more cells with a disclosed isolated nucleic acid molecule or a disclosed vector, wherein expression of the nucleic acid molecule improves mRNA transcript stability and/or improves mRNA transcript translation efficiency.

In an aspect of method of improving vaccine formulation, a transgene can comprise any gene disclosed herein (discussed supra in connection with the disclosed Compositions) or any gene that is missing, deficient, and/or mutated or any gene that is producing a deficient or mutated protein or enzyme. By knowing what disease or disorder is affecting the subject, the skilled person can identify the relevant gene or genes.

In an aspect, the one or more cells can be in a subject. In an aspect, a subject can have a disclosed genetic disease or disorder. I n an aspect, a subject can have a disease or disorder. In an aspect, a disease or disorder can be any disease or disorder disclosed herein. In an aspect, a disease or disorder can comprise any disease or disorder caused by a disclosed gene or a missing, deficient, and/or mutant gene. In an aspect, a subject can be a subject in need of treatment of a disclosed disease or disorder (e.g., a genetic disease or disorder). In an aspect, a subject can have or be suspected of having a disease or disorder that can be treated with gene therapy.

In an aspect, a disclosed vaccine can be administered to a subject in need thereof. In an aspect, a disclosed vaccine formulation, whether a protein-based vaccine, nucleic acid-based vaccine or virus-based vaccine, can include additional ingredients such as excipients or adjuvants.

### H. Kits

Disclosed herein is a kit comprising a disclosed isolated nucleic acid molecule, a disclosed vector, a disclosed pharmaceutical formulation, or any combination thereof. In an aspect, a kit can comprise a disclosed isolated nucleic acid molecule, a disclosed vector, a disclosed pharmaceutical formulation, a disclosed RNA therapeutic, or a combination thereof, and one or more agents. “Agents” and “Therapeutic Agents” are known to the art and are described supra.

In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to a missing, deficient, and/or mutant protein or enzyme.

In an aspect, a disclosed kit can comprise at least two components constituting the kit. Together, the components constitute a functional unit for a given purpose (such as, for example, treating a subject diagnosed with or suspected of having a disease or disorder). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed isolated nucleic acid molecule, a disclosed vector, a disclosed pharmaceutical formulation, a disclosed RNA therapeutic, or a combination thereof, and a label or package insert with instructions for use. In an aspect, suitable containers include, for example, bottles, vials, syringes, blister pack, etc. The containers can be formed from a variety of materials such as glass or plastic. The container can hold a disclosed isolated nucleic acid molecule, a disclosed vector, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed isolated nucleic acid molecule, a disclosed vector, a disclosed pharmaceutical formulation, a disclosed RNA therapeutic, or a combination thereof can be used for treating, preventing, inhibiting, and/or ameliorating a disease or disorder or complications and/or symptoms associated with a disease or disorder. A kit can comprise additional components necessary for administration such as, for example, other buffers, diluents, filters, needles, and syringes.

In an aspect, a disclosed kit can be used to increase the stability or half-life of an mRNA transcript or RNA molecule. In an aspect, a disclosed kit can be used to increase the translation efficiency of an mRNA transcript or RNA molecule. In an aspect, a disclosed kit can be used to enhance gene therapy. In an aspect, a disclosed kit can be used to treat and/or prevent a disease or disorder.

### I. Miscellaneous

Disclosed herein is a nucleic acid molecule comprising at least one of the following: the partial or complete sequence of one or more Flavivirus 3′ untranslated region (3′ UTR) element(s); the partial or complete sequence of one or more Flavivirus subgenomic Flavivirus RNA (sfRNA) element(s); the partial or complete sequence of one or more Flavivirus XRN1-resistant RNA (xrRNA) element(s); the partial or complete sequence of one or more Flavivirus sfRNA dumbbell (DB) RNA element(s); and the partial or complete sequence of one or more Flavivirus 3′ stem loop (3′ SL) element(s).

In an aspect, a disclosed nucleic acid molecule can comprise sequence selected from Dengue virus serotype 1-4 (DENV1-4), Zika virus (ZIKV), Japanese encephalitis virus (JEV), West Nile virus (WNV), Yellow fever virus (YFV), Murray Valley encephalitis virus (MVEV), another Flavivirus, a serotype of a Flavivirus, any Flavivirus disclosed herein, or any combination thereof. In an aspect, a disclosed nucleic acid molecule can comprise the full sequence or a partial sequence set forth in any one of SEQ ID NO:08-SEQ ID NO: 19.

Disclosed herein is a nucleic acid molecule compromising a nucleic acid molecule comprising at least one of the following: the partial or complete sequence of one or more Flavivirus 3′ untranslated region (3′ UTR) element(s); the partial or complete sequence of one or more Flavivirus subgenomic Flavivirus RNA (sfRNA) element(s); the partial or complete sequence of one or more Flavivirus XRN1-resistant RNA (xrRNA) element(s); the partial or complete sequence of one or more Flavivirus sfRNA dumbbell (DB) RNA element(s); and the partial or complete sequence of one or more Flavivirus 3′ stem loop (3′ SL) element(s), wherein the nucleic acid molecule is in a DNA template.

Disclosed herein is a nucleic acid molecule compromising a nucleic acid molecule comprising at least one of the following: the partial or complete sequence of one or more Flavivirus 3′ untranslated region (3′ UTR) element(s); the partial or complete sequence of one or more Flavivirus subgenomic Flavivirus RNA (sfRNA) element(s); the partial or complete sequence of one or more Flavivirus XRN1-resistant RNA (xrRNA) element(s); the partial or complete sequence of one or more Flavivirus sfRNA dumbbell (DB) RNA element(s); and the partial or complete sequence of one or more Flavivirus 3′ stem loop (3′ SL) element(s), wherein the nucleic acid molecule was in a DNA template, wherein upon transcription, the nucleic acid molecule is present within an RNA molecule.

Disclosed herein in a nucleic acid molecule compromising nucleic acid molecule compromising a nucleic acid molecule comprising at least one of the following: the partial or complete sequence of one or more Flavivirus 3′ untranslated region (3′ UTR) element(s); the partial or complete sequence of one or more Flavivirus subgenomic Flavivirus RNA (sfRNA) element(s); the partial or complete sequence of one or more Flavivirus XRN1-resistant RNA (xrRNA) element(s); the partial or complete sequence of one or more Flavivirus sfRNA dumbbell (DB) RNA element(s); and the partial or complete sequence of one or more Flavivirus 3′ stem loop (3′ SL) element(s), wherein the nucleic acid molecule is in a RNA template.

In an aspect, a disclosed RNA molecule can encode a protein. In an aspect, a disclosed RNA molecule can encode an RNA element, wherein RNA element can comprise lncRNA(s), siRNA(s), shRNA(s), sgRNA(s), circular RNA(s), snoRNA(s), miRNA(s), other functional non-coding RNA element(s), or any combination thereof.

In an aspect, a disclosed nucleic acid molecule can comprise one or more copies of a disclosed Flavivirus genetic element(s), wherein the Flavivirus genetic element(s) is located 3′ of the open reading frame (ORF) of the transgene.

Disclosed herein is a method comprising using a nucleic acid molecule compromising a nucleic acid molecule comprising at least one of the following: the partial or complete sequence of one or more Flavivirus 3′ untranslated region (3′ UTR) element(s); the partial or complete sequence of one or more Flavivirus subgenomic Flavivirus RNA (sfRNA) element(s); the partial or complete sequence of one or more Flavivirus XRN1-resistant RNA (xrRNA) element(s); the partial or complete sequence of one or more Flavivirus sfRNA dumbbell (DB) RNA element(s); and the partial or complete sequence of one or more Flavivirus 3′ stem loop (3′ SL) element(s), to increase the stability or half-life of an RNA molecule.

Disclosed herein is a method comprising using a nucleic acid molecule compromising a nucleic acid molecule comprising at least one of the following: the partial or complete sequence of one or more Flavivirus 3′ untranslated region (3′ UTR) element(s); the partial or complete sequence of one or more Flavivirus subgenomic Flavivirus RNA (sfRNA) element(s); the partial or complete sequence of one or more Flavivirus XRN1-resistant RNA (xrRNA) element(s); the partial or complete sequence of one or more Flavivirus sfRNA dumbbell (DB) RNA element(s); and the partial or complete sequence of one or more Flavivirus 3′ stem loop (3′ SL) element(s), to increase the translation efficiency of a mRNA transcript or an RNA molecule.

In an aspect, a disclosed nucleic acid molecule can be packaged within a viral vector derived from adeno-associated virus, lentivirus, adenovirus, or any other recombinant version.

In an aspect, a disclosed nucleic acid molecule can be expressed in a cell.

Disclosed herein is a method, comprising administering to a mammalian subject a nucleic acid molecule compromising a nucleic acid molecule comprising at least one of the following: the partial or complete sequence of one or more Flavivirus 3′ untranslated region (3′ UTR) element(s); the partial or complete sequence of one or more Flavivirus subgenomic Flavivirus RNA (sfRNA) element(s); the partial or complete sequence of one or more Flavivirus XRN1-resistant RNA (xrRNA) element(s); the partial or complete sequence of one or more Flavivirus sfRNA dumbbell (DB) RNA element(s); and the partial or complete sequence of one or more Flavivirus 3′ stem loop (3′ SL) element(s), and expressing the nucleic acid molecule.

### VII. EXAMPLES

Following transduction of target cells, the AAV genome is transcribed, producing AAV transgene mRNA that is then translated into the final protein product. While significant effort to identify and engineer promoter elements for AAV vectors has been conducted, little effort has been focused on RNA elements within the transgene mRNA that either enhance mRNA stability or increase mRNA translation efficiency, resulting in more transgene product. The presence of stable RNA structures in either the mRNA 5′ or 3′ untranslated regions (UTRs) can significantly improve the stability of an mRNA, leading to enhanced translation. Alternatively, RNA secondary structures within the mRNA UTRs can promote the recruitment of specific cellular factors that promote enhanced translation of that mRNA. Such functions of specific RNA structures have been characterized in several viral RNA elements, prompting the possibility that addition of such viral RNA elements to an AAV vector could have beneficial effects on AAV transgene expression.

Many RNA viruses utilize novel and creative RNA-related mechanisms to evade host immune responses and efficiently express their genetic information. The Flavivirus genus of RNA viruses are small-enveloped viruses with a positive-stranded ˜11 kb mRNA that lacks a poly(A) tail. Rather, the 3′ terminus of Flavivirus genomic RNAs (gRNA) consists of a highly conserved, complex RNA secondary structure. In Flavivirus-infected cells, the gRNA is partially degraded by the 5′ to 3′ exonuclease XRN1 and results in the production of a subgenomic Flavivirus RNA (sfRNA) that consists of the last ˜300-400 nucleotides of the gRNA. There are three major structural RNA elements present in the sfRNA: (i) XRN1-resistant RNA structures (xrRNAs) at the 5′ end, (ii) dumbbell RNA structures (DBs) in the center, and (iii) a 3′ terminal RNA stem loop (3′ SL). The xrRNA structures block XRN1 exonuclease activity, resulting in sfRNA production. The DB structures also limit XRN1 activity, and depending on the species, the Flavivirus 3′ UTR can contain more than one xrRNA and/or DB RNA element, resulting in the production of multiple sfRNA species.

Flavivirus sfRNAs accumulate throughout infection and have several functions. The xrRNA structures have been shown to block the activity of multiple 5′-3′ exonucleases and sequester XRN1 during infection. This has been shown to alter the stability of several host mRNAs. Multiple studies have shown that the sfRNA binds a variety of host proteins, some of which have been characterized as having roles in viral replication and translation, including Dicer and Ago2, resulting in impairment of host cell miRNA metabolism. Additionally, there is growing evidence that the sfRNAs are involved in modulating the host immune response and can block interferon-s and RIG-I pathways and interferon-stimulated gene expression.

Given the variety of cellular processes in which they are involved, sfRNAs show the potential to have beneficial effects on AAV transgene expression when added to a recombinant AAV vector. The Examples that follow are illustrative of specific aspects of the invention, and various uses thereof. They set forth for explanatory purposes only and are not to be taken as limiting the invention.

### Example 1

### Sequence Alignment Confirmed Conservation of Subgenomic Flaviviral RNAs

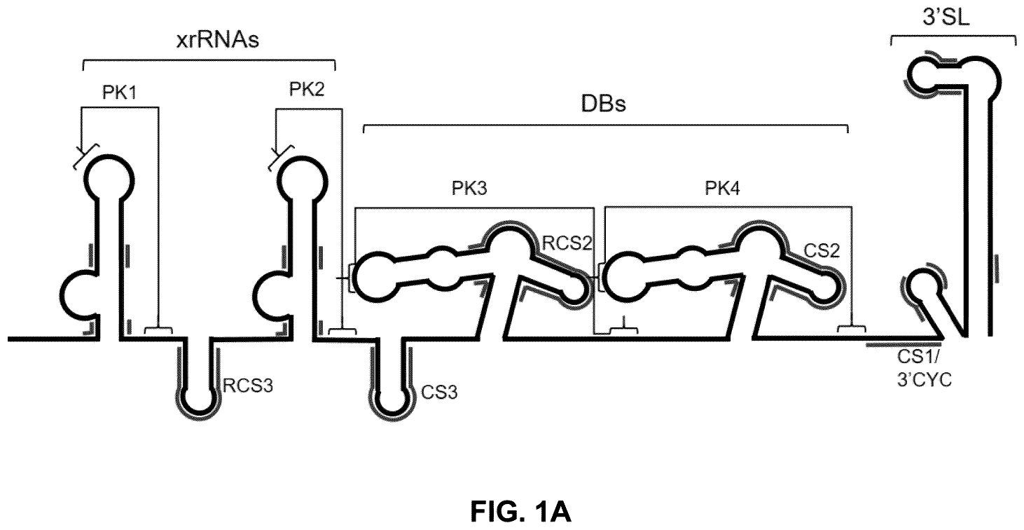

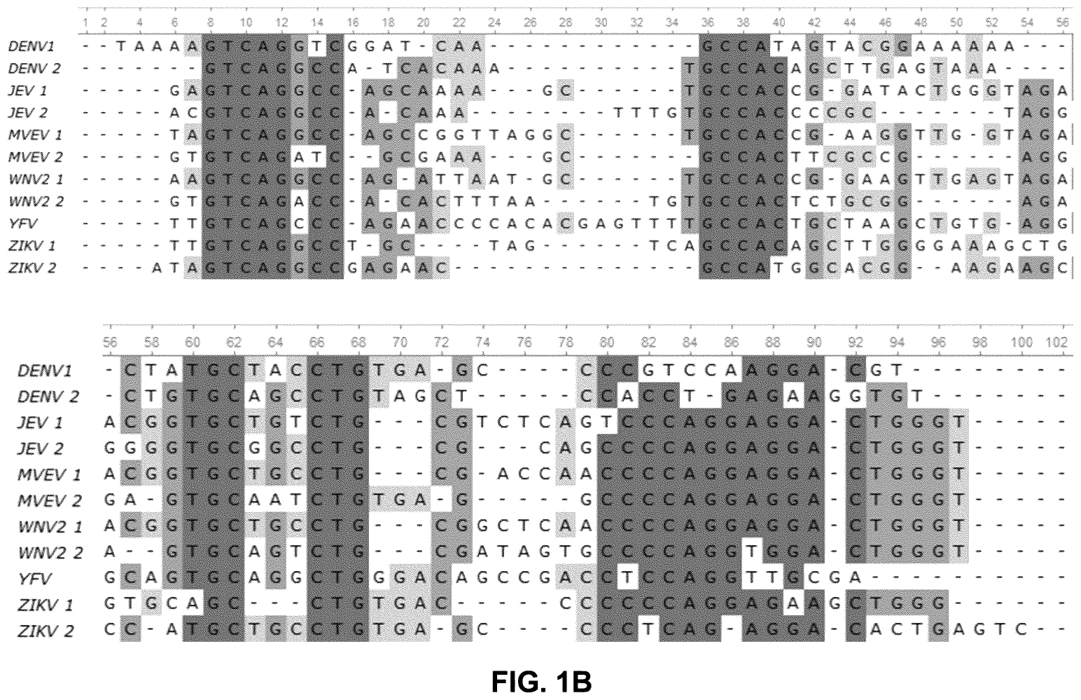

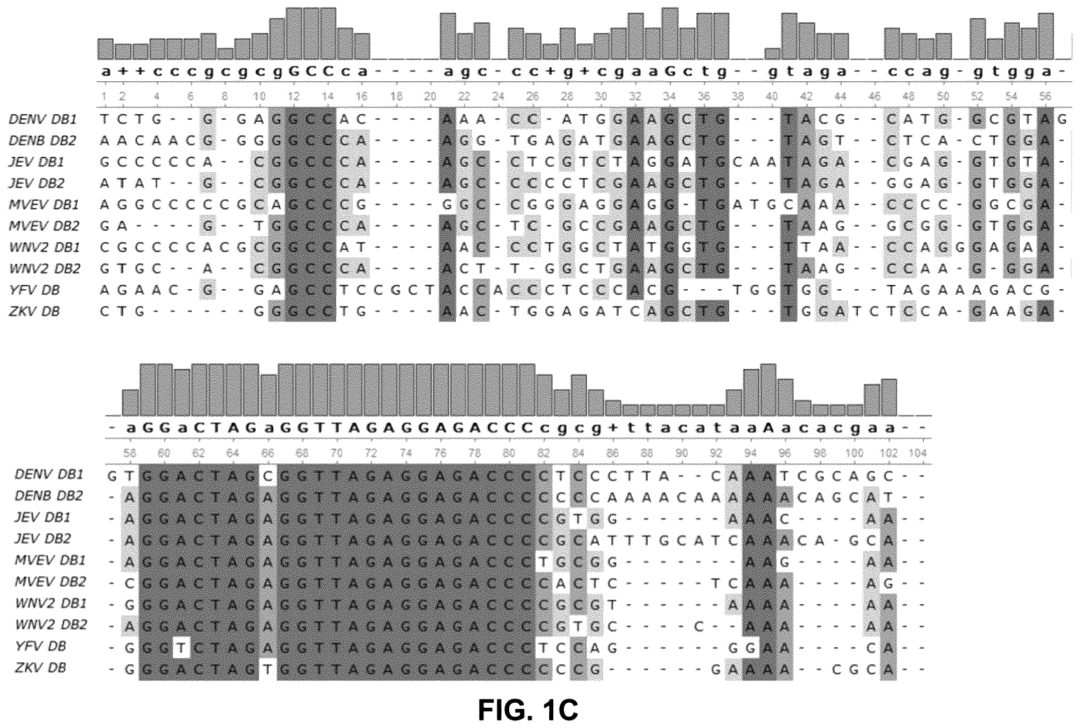

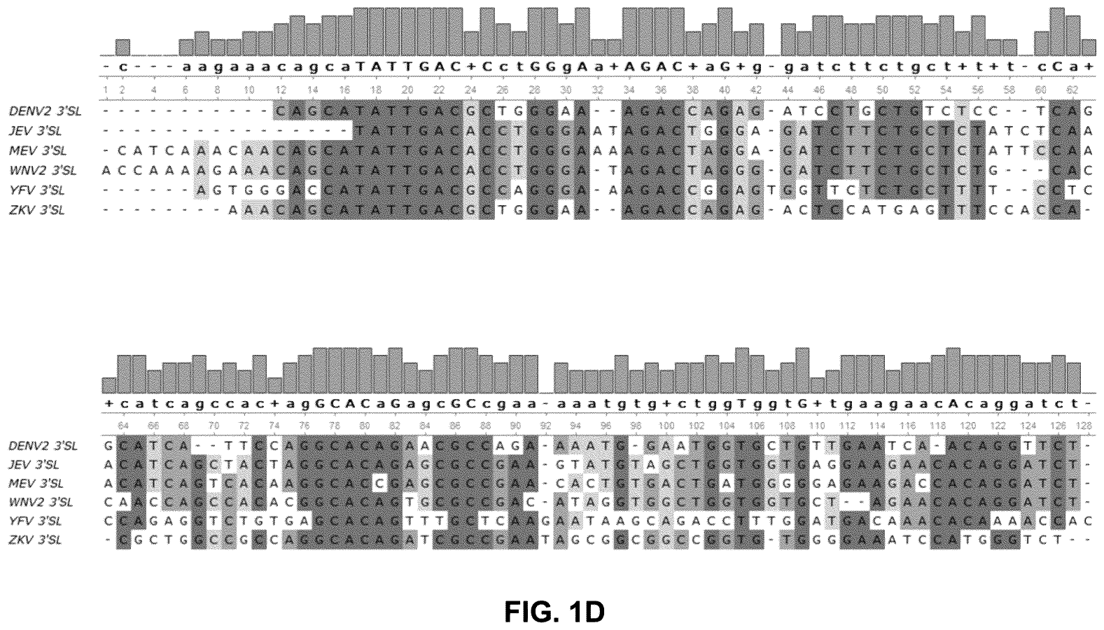

The subgenomic Flaviviral RNAs are conserved across various Flaviviruses. FIG. 1A shows the secondary structure of the DENV2 sfRNA including two xRNAs, two DBs, and a 3′ SL. Various structural elements are noted. CS1/3′CYC indicates conserved sequence 1 with the 3′ cyclization sequence. CS2 indicates conserved sequence 2 while CS3 indicates conserved sequence. FIG. 1B shows the sequence conservation of the xrRNA element of subgenomic Flaviviral RNAs for DENV 1 and DENV 2, JEV 1 and JEV 2, MVEV 1 and MVEV 2, WNV2 1 and WNV 2, YFV, and ZIKV 1 and ZIKV 2. FIG. 1C shows the sequence conservation of the DB element aspect of subgenomic Flaviviral RNAs for DENV, JEV, MVEV, WNV2, YFV, and ZKV. FIG. 1D show the sequence conservation of the 3′ SL of subgenomic Flaviviral RNAs for DENV2, JEV, MVEV, WNV2, YFV, and ZIKV.

### Example 2

### Subgenomic Flaviviral XRN1 Restricted AAV Transduction

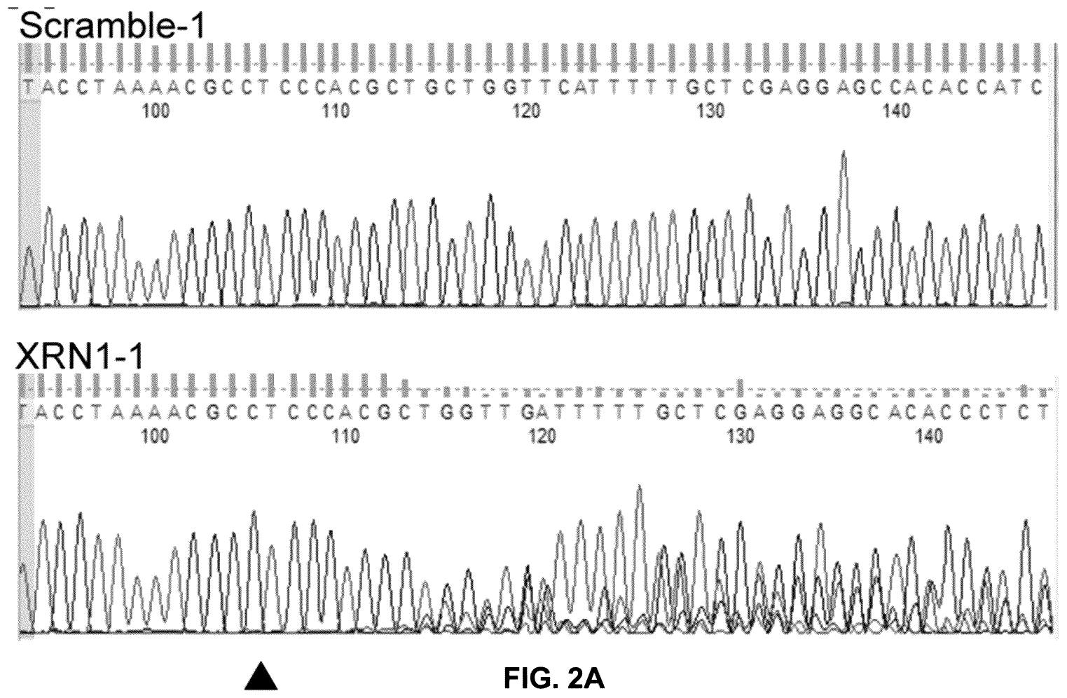

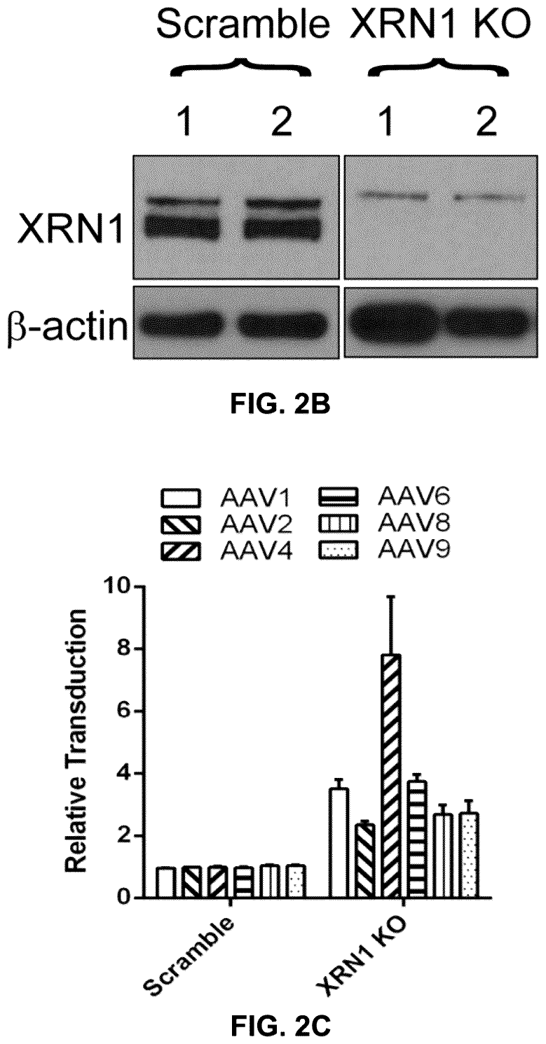

Cellular 5′-3′ exoribonuclease 1 (XRN1) is best known for its role as a decay factor. In Flavivirus-infected cells, XRN1 partially degrades the 3′ terminus of Flavivirus genomic RNA, which results in the production of subgenomic Flavivirus RNA (sfRNA). The generation of sfRNA includes the production of XRN1-resistant RNA structures (xrRNAs), dumbbell RNA structures (DBs), and a 3′ terminal RNA stem loop (3′ SL). FIG. 2A-FIG. 2B show that XRN1 restricts AAV transduction. Specifically, FIG. 2A shows Huh7 cells following infection with lentivirus packaging Cas9 and either a Scramble gRNA (top) or gRNA against the XRN1 gene (bottom). Following antibiotic selection, sequencing at the XRN1 locus confirmed Cas9 activity with the XRN1-1 gRNA. The arrow denotes the predicted Cas9 cleavage site. FIG. 2B shows Western blotting performed on two independent Scramble and XRN1 KO clonal cell lines for XRN1 (top) and R-actin (bottom). FIG. 2C shows the quantitation of transgene expression from different AAV serotypes in XRN1 KO cells.

### Example 3

### Subgenomic Flaviviral RNAs Increased AAV Transduction Efficiency

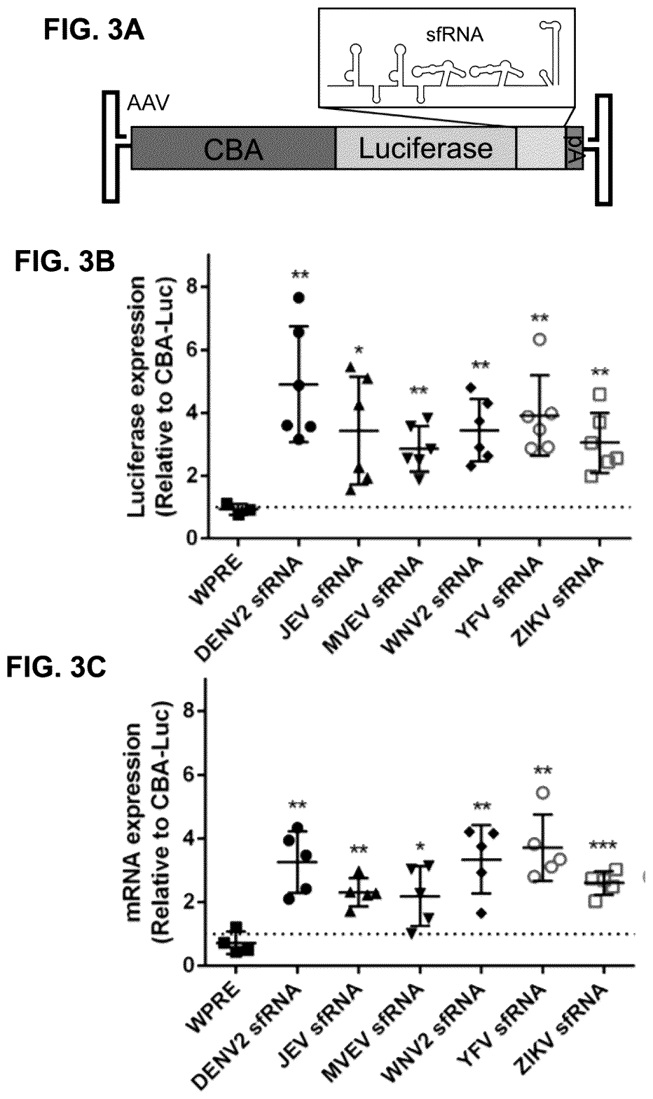

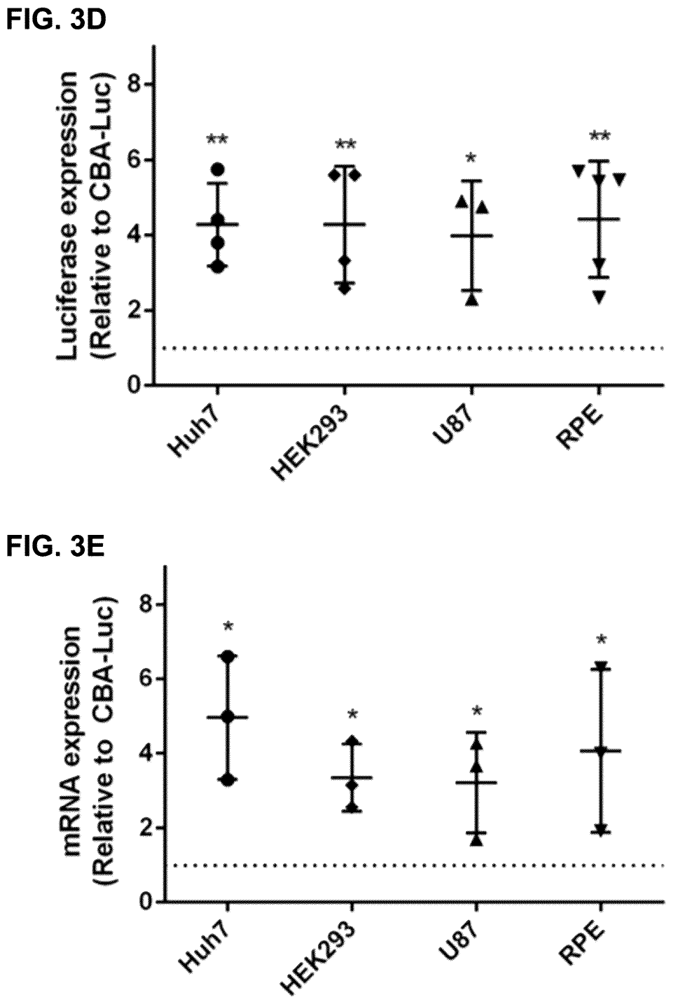

The effect of subgenomic Flaviviral RNAs (sfRNAs) was examined. FIG. 3A-FIG. 3E show that sfRNAs increased AAV transduction efficiency. FIG. 3A shows a diagram of an experimental construct. Here, a luciferase transgene with SV40 poly-A signal and driven by the CBA promoter was placed between AAV2 ITRs. SfRNA (or WPRE) sequences were placed as the 3′ UTR of the luciferase mRNA. FIG. 3B shows luciferase expression in Huh7 cells transduced with the indicated constructs at 10,000 vg/cell and harvested at 3 days post-transduction. Lysates were measured for Relative Luciferase Expression (RLUs). FIG. 3C shows RNA from replicate samples was used to perform qRT-PCR for luciferase mRNAs. FIG. 3D shows mRNA expression in various cell lines were transduced with 10,000 vg/cell of the DENV2 sfRNA construct and harvested at 3 days post-transduction. Lysates were measured for Relative Luciferase Expression (RLUs). FIG. 3E shows RNA from replicate samples was used to perform qRT-PCR for luciferase mRNAs. For all graphs, samples are plotted relative to a CBA-Luc transgene with no additional 3′ UTR. Student's t-test was performed to test for statistical significance. Where indicated for FIG. 3B-FIG. 3E, * =p<0.05; ** =p<0.005; *** =p<0.0005; **** =p<0.00005.

### Example 4

### The Effect of DENV2 sfRNA on AAV Transduction Was Context-Dependent

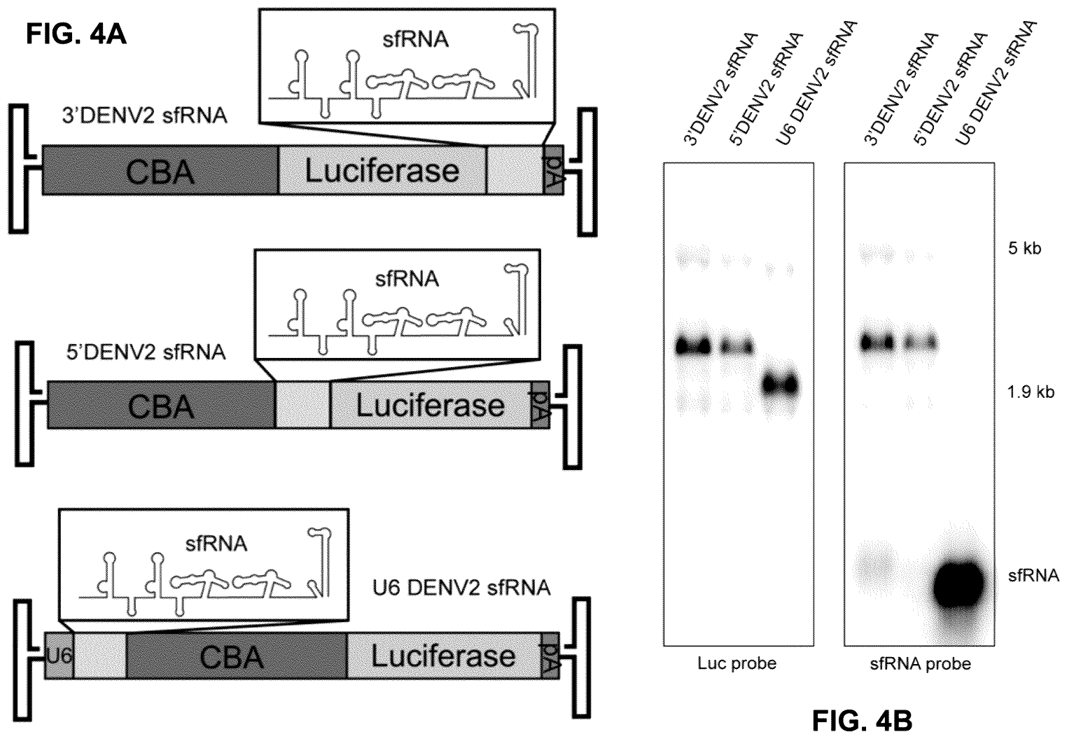

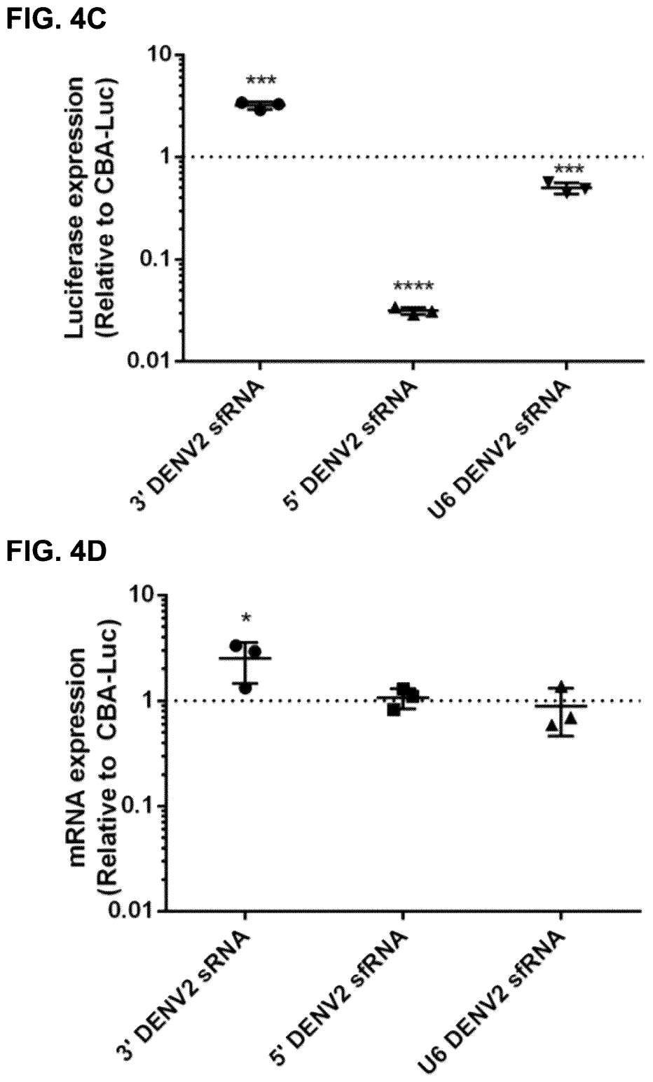

The location of sfRNA from Denv2 in a construct was examined. FIG. 4A-FIG. 4C shows that the effect of DENV2 sfRNA on AAV transduction was context-dependent. FIG. 4A shows a construct in which the DENV2 sfRNA was placed in the 5′ of the transgene (top construct), 3′ of the transgene (middle construct), or as a separate and U6-driven RNA packaged in the same AAV genome (lower construct). FIG. 4B shows northern blots of HEK293 cells transfected with the various constructions in FIG. 4A and then harvested 3 days post-transfection. The northern blots were probed for either luciferase or sfRNA sequences. FIG. 4C-FIG. 4D show Huh7 cells transduced with the indicated constructs at 10,000 vg/cell and then harvested 3 days post-transfection. FIG. 4C shows lysates that were measured for Relative Luciferase Expression (RLUs) while FIG. 4D shows RNA from replicate samples that was used to perform qRT-PCR for luciferase mRNAs. Data was relative to a CBA-Luc transgene with no additional 3′ UTR. For FIG. 4C-FIG. 4D, Student's t-test was performed to test for statistical significance, and where indicated, * =p<0.05; ** =p<0.005; *** =p<0.0005; **** =p<0.00005.

### Example 5

### The polyA Tail was Necessary for Increased Transgene Expression

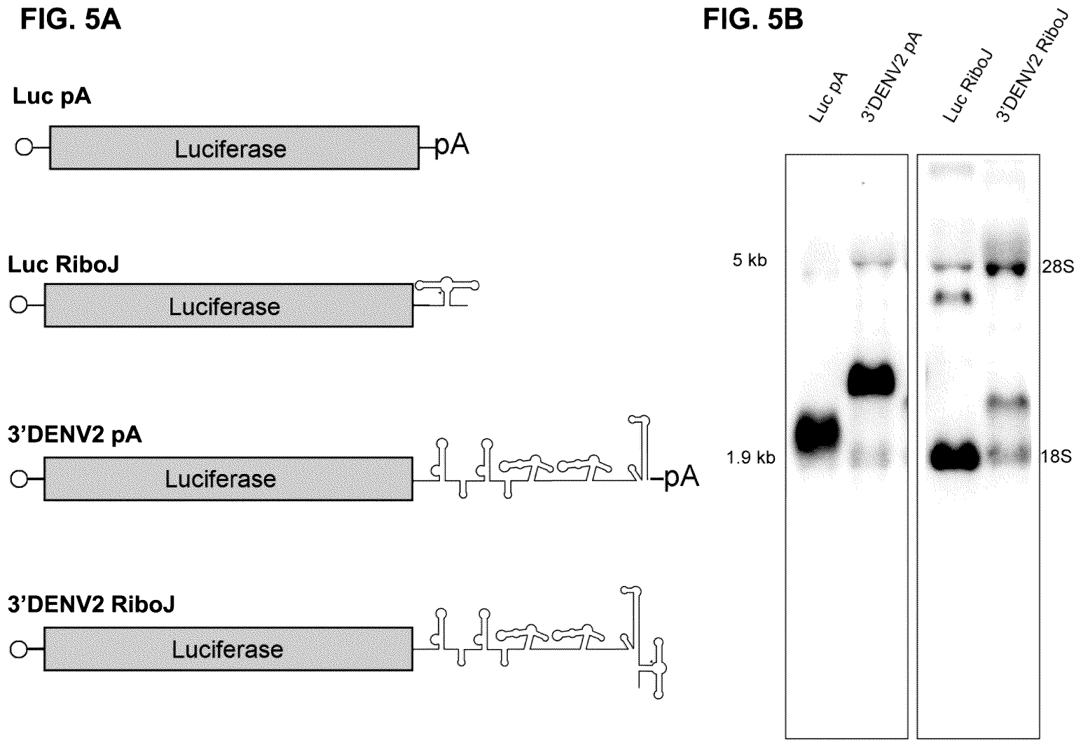

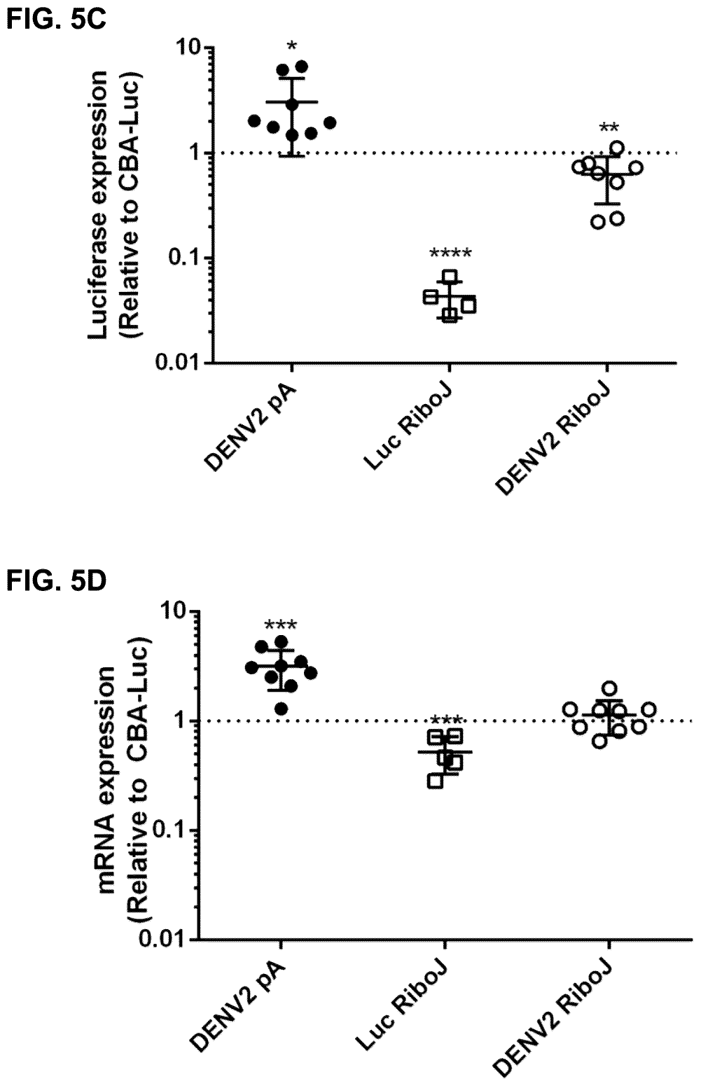

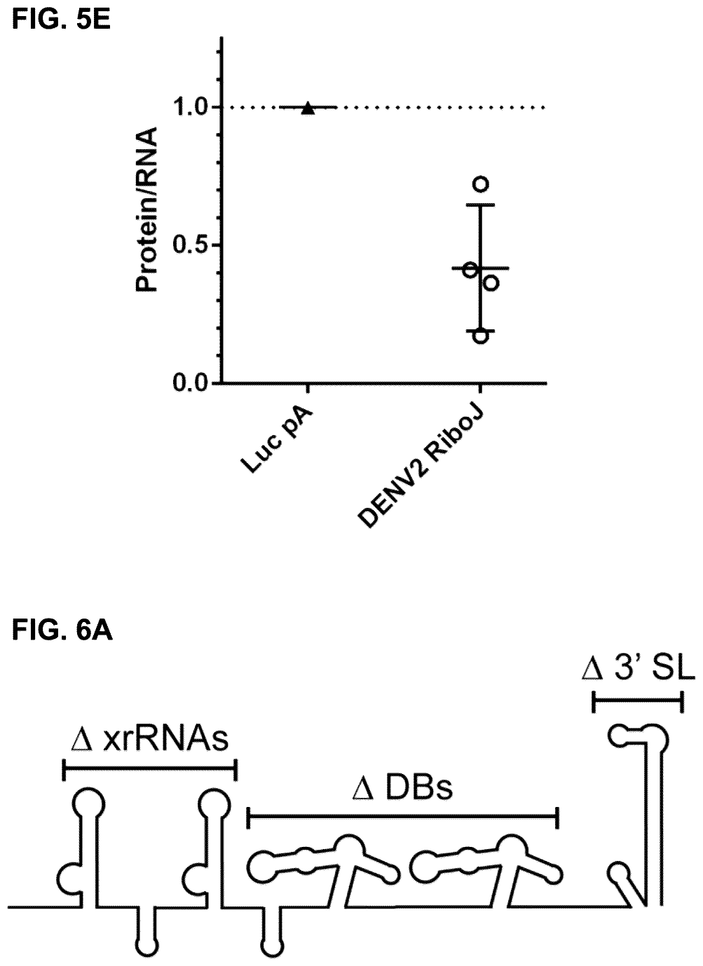

The significance of a polyA tail for transgene expression was examined. FIG. 5A-FIG. 5E shows that the presence of polyA tail was necessary for increased transgene expression. FIG. 5A shows a diagram of luciferase mRNAs with either a poly-A or RiboJ (hammerhead ribozyme) 3′ end with and without the DENV2 sfRNA. FIG. 5B shows northern blots of HEK293 cells following transfection with the indicated constructs and then harvested 3 days post-transfection. The northern blots were probed for luciferase sequences. FIG. 5C shows Huh7 cells transduced with the indicated constructs at 10,000 vg/cell and then harvested at 3 days post-transduction. Lysates were measured for Relative Luciferase Expression (RLUs). FIG. 5D shows qRT-PCR for luciferase mRNAs using replicate samples. Data is shown relative to a CBA-Luc transgene with no additional 3′ UTR. FIG. 5E shows the ratio of protein and RNA expression calculated for the DENV2 RiboJ construct relative to CBA-Luc with a poly-A tail. Student's t-test was performed to test for statistical significance. In FIG. 5C-FIG. 5E, where indicated, * =p<0.05; ** =p<0.005; *** =p<0.0005; **** =p<0.00005.

### Example 6

### The Dumbbell Elements were Necessary and Sufficient to Increase AAV Transduction Efficiency

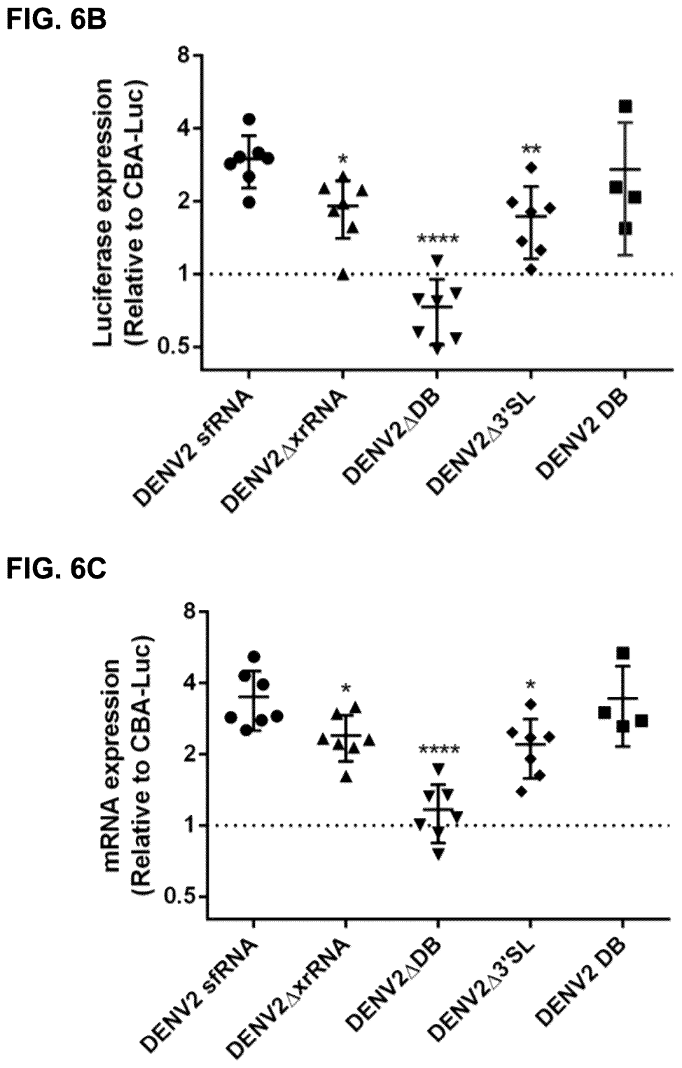

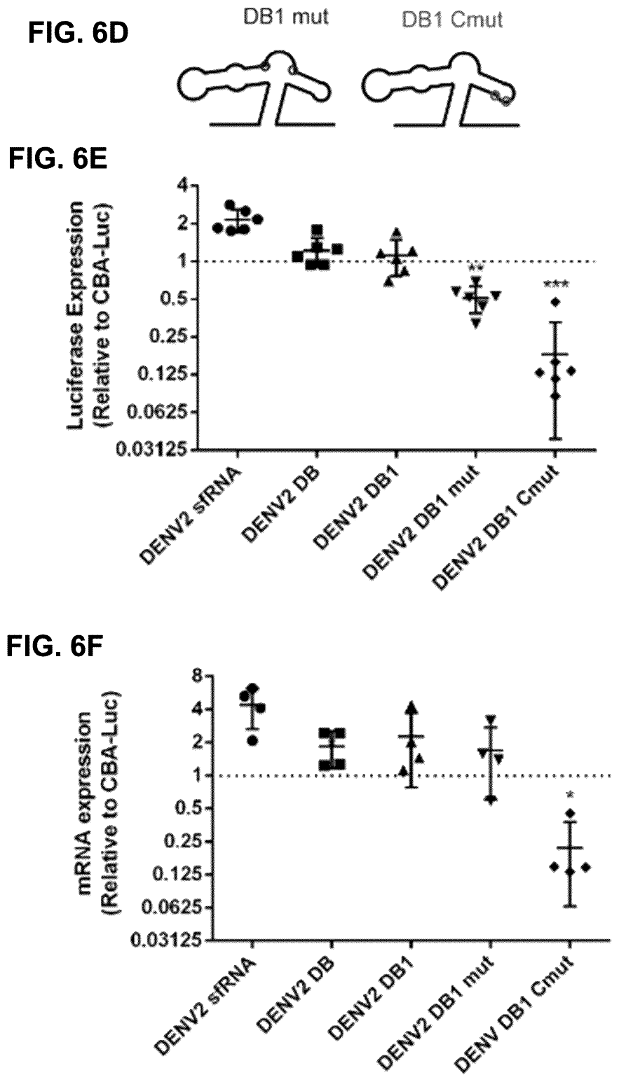

Whether any specific sfRNA element was necessary and sufficient to increase the efficiency of AAV transduction was evaluated. FIG. 6A-FIG. 6F shows that the DENV2 DB elements were necessary and sufficient to increase AAV transduction efficiency. FIG. 6A shows a diagram of the DENV2 sfRNA with the indicated deletions of RNA structural elements. FIG. 6B shows Huh7 cells transduced with the indicated constructs at 10,000 vg/cell and harvested at 3 days post-transduction. Lysates were measured for Relative Luciferase Expression (RLUs). FIG. 6C shows qRT-PCR for luciferase mRNAs performed on replicate samples. Data is shown relative to a CBA-Luc transgene with no additional 3′ UTR. FIG. 6D shows mutations in the first DB of DENV2 were created at the indicated locations. Huh7 cells were transduced with the indicated constructs at 10,000 vg/cell and harvested at 3 days post-transduction. FIG. 6E shows Relative Luciferase Expression (RLUs) from lysates and FIG. 6F shows qRT-PCR for luciferase mRNAs performed on replicate samples. Data is shown relative to a CBA-Luc transgene with no additional 3′ UTR. Student's t-test was performed to test for statistical significance. In FIG. 6B-FIG. 6C and FIG. 6E-FIG. 6F, where indicated, * =p<0.05; ** =p<0.005; *** =p<0.0005; **** =p<0.00005.

### Example 7

### The Role of sfRNA on AAV Transduction Was Context-Dependent

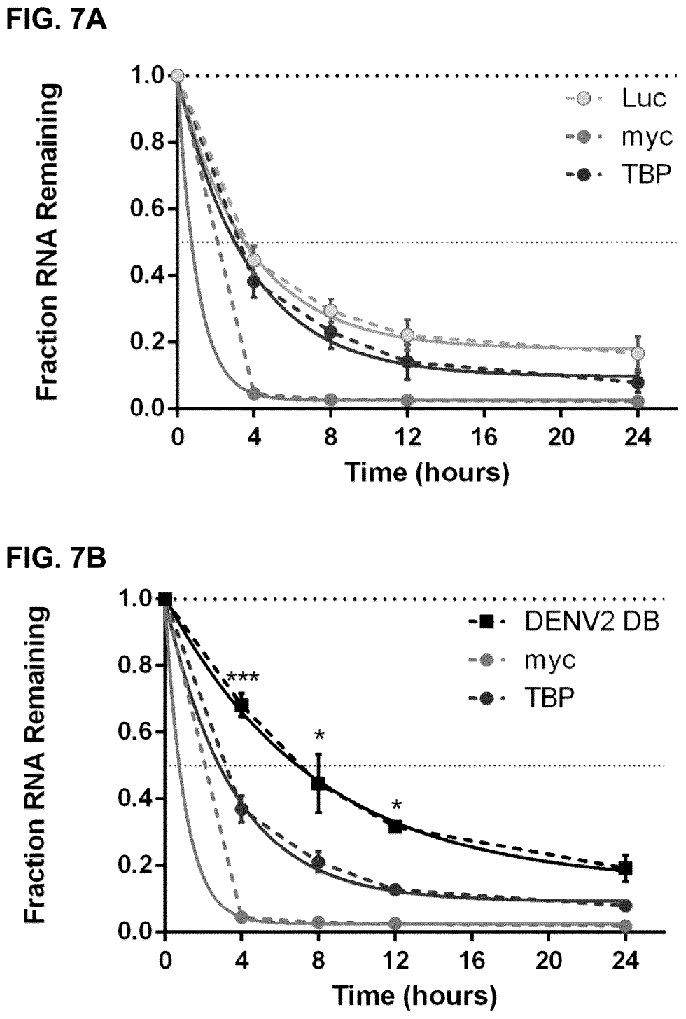

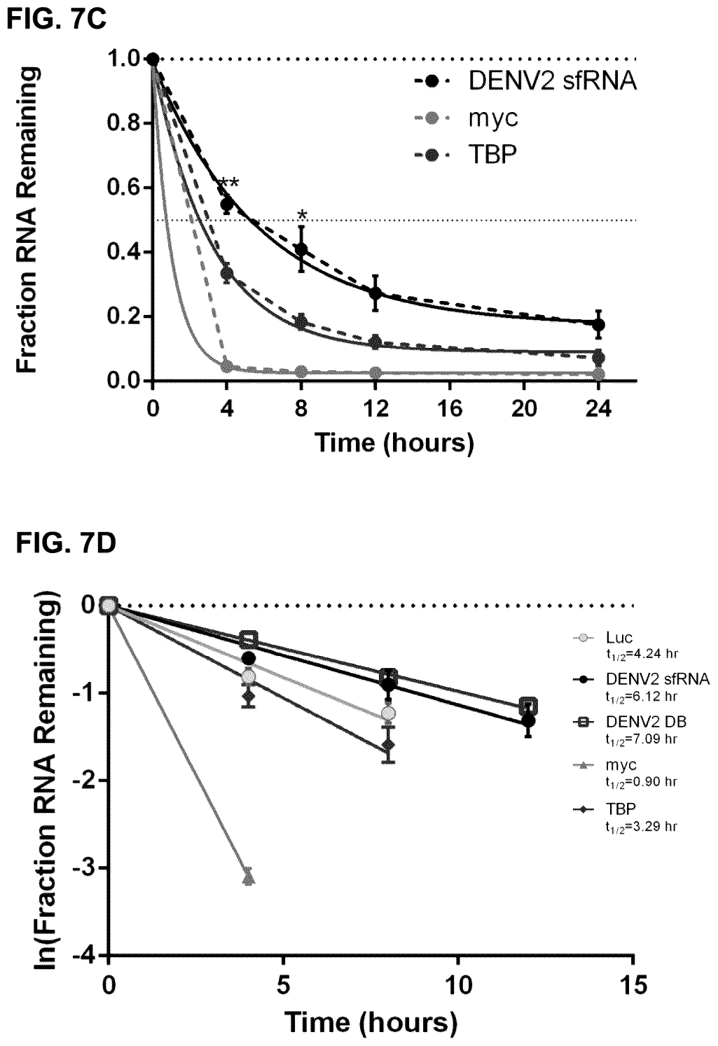

The effect of various sfRNA elements on AAV transduction was examined. FIG. 7A-FIG. 7D show that DENV2 sfRNA and DB elements increase mRNA half-life. HEK293 cells were transduced with the indicated constructs at 10,000 vg/cell. At 3 days post transduction, Actinomycin D was added to cells and RNA collected at the indicated time points. RNA levels were measured by qRT-PCR, and graphed relative to the level at 0 hours and normalized to GAPDH mRNA levels. FIG. 7A shows the fraction RNA remaining for 3 constructs, FIG. 7B shows that the DENV2 DB increased the mRNA half-life while FIG. 7C shows that the DENV2 sfRNA increased the mRNA half-life. FIG. 7D shows that DENV2 sfRNA and DENV2 DB generated RNA having longer half-lives. Student's t-test was performed to test for statistical significance. In FIG. 7A-FIG. 7D, where indicated, * =p<0.05; ** =p<0.005; *** =p<0.0005; **** =p<0.00005. Here, Luc refers to luciferase, Myc refers to cellular c-myc (Myc) mRNA, and TBP refers to cellular TATA Box-binding protein (TBP) mRNA.

1. An isolated nucleic acid molecule, comprising:
a nucleic acid sequence encoding a transgene; and
a nucleic acid sequence encoding one or more Flavivirus genetic elements,
wherein the Flavivirus genetic elements comprise one or more Flavivirus 3′ untranslated regions (3′ UTR), one or more subgenomic Flavivirus RNA (sfRNA) elements, one or more Flavivirus XRN1-resistant RNA (xrRNA) elements, one or more Flavivirus dumbbell (DB) RNA elements, one or more Flavivirus 3′ stem loop (3′ SL) elements, or any combination thereof.

2.-3. (canceled)

4. The isolated nucleic acid molecule of claim 1, wherein the 3′ UTR comprises a sequence having at least 80%, 85%, 86%, 87%, 88%, 89%, 90%, 91%, 92%, 93%, 94%, 95%, 96%, 97%, 98%, 99% identity to the sequence set forth in any one of SEQ ID NO:08-SEQ ID NO: 19.

5. The isolated nucleic acid molecule of claim 1, wherein the one or more 3′ SL elements comprise the sequence set forth in SEQ ID NO:01.

6. The isolated nucleic acid molecule of claim 1, wherein the one or more xrRNA elements comprise the sequence set forth in SEQ ID NO:02 or SEQ ID NO:03.

7. The isolated nucleic acid molecule of claim 1, wherein the one or more sfRNA dumbbell (DB) RNA elements comprise the sequence set forth in SEQ ID NO:04 or SEQ ID NO:05.

8. The isolated nucleic acid molecule of claim 1, wherein the Flavivirus genetic elements are derived from Dengue virus type 1 (DENV1), Dengue virus type 2 (DENV2), Dengue virus type 3 (DENV3), Dengue virus type 4 (DENV4), Japanese encephalitis virus (JEV), Murray Valley encephalitis virus (MVEV), Powassan virus (POWV), St. Louis encephalitis virus (SLEV), Tick-borne Encephalitis (TBEV), West Nile virus (WNV), Zika virus (ZIKV), Yellow fever virus (YFV), or any combination thereof.

9. The isolated nucleic acid molecule of claim 1, wherein the transgene encodes a polypeptide or an RNA.

10. (canceled)

11. The isolated nucleic acid molecule of claim 9, wherein the RNA comprises lncRNA, siRNA, shRNA, sgRNA, circular RNA, snoRNA, miRNA, or any combination thereof.

12. The isolated nucleic acid molecule of claim 1, further comprising a nucleic acid sequence encoding a promoter operably linked to the transgene.

13. The isolated nucleic acid molecule of claim 12, wherein the nucleic acid sequence encoding the one or more Flavivirus genetic elements is positioned 3′ of the transgene ORF, 5′ of the transgene ORF, or 3′ of the promoter.

14. A vector, comprising: the isolated nucleic acid molecule of claim 1.

15. The vector of claim 14, wherein the vector is an adenovirus vector, an adenovirus-associated (AAV) vector, or a lentivirus vector.

16.-18. (canceled)

19. A method of treating and/or preventing a disease or disorder, comprising:
administering to a subject in need thereof a therapeutically effective amount of the vector of claim 14 Error! Reference source not found.,
wherein, following expression of the nucleic acid molecule, the transgene mRNA transcript stability is improved and/or the transgene mRNA transcript translation efficiency is improved.

20. The method of claim 19, wherein the subject has a genetic disease or disorder.

21. The method of claim 19, wherein the therapeutically effective amount of the vector comprises about 1×10¹⁰ vg to about 2×10¹⁴ vg.

22. The method of claim 19, wherein the vector is administered via intravenous administration.

23.-39. (canceled)

40. The method of claim 20, where the transgene comprises a missing gene, a deficient gene, or a gene having a mutant protein or enzyme product.

41. The method of claim 40, wherein the functionality and/or structural integrity of the missing gene, the deficient gene, and/or the gene having a mutant protein or enzyme is restored in the subject.

42. The method of claim 19, further comprising restoring in the subject one or more aspects of cellular homeostasis and/or cellular functionality and/or metabolic dysregulation.

43. The method of claim 42, wherein restoring in the subject one or more aspects of cellular homeostasis and/or cellular functionality comprises (i) correcting cell starvation in one or more cell types; (ii) normalizing aspects of the autophagy pathway; (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof.
